(19) United States
(12) Patent Application Publication
(10) Pub. No.: US 2015/0025060 A1
(43) Pub. Date: Jan. 22, 2015

(54) Foamable Compositions and Kits Comprising One or More of a Channel Agent, a Cholinergic Agent, A nitric Oxide Donor and Related Agents and Their Uses

(71) Applicant: Foamix Pharmaceuticals Ltd., Rehovot (IL)

(72) Inventors: Dov Tamarkin, Macabim (IL); Meir Eini, Ness Ziona (IL); Doron Friedman, Karmei Yosef (IL); Tal Berman, Rishon Le Ziyyon (IL); Alex Besonov, Rehovot (IL)

(21) Appl. No.: 14/448,670

(22) Filed: Jul. 31, 2014

Related U.S. Application Data
(63) Continuation of application No. 11/767,442, filed on Jun. 22, 2007.
Application No. 11/767,442 is a continuation-in-part of application No. 10/911,367, filed on Aug. 4, 2004.
Application No. 11/767,442 is a continuation-in-part of application No. 10/532,618, filed on Dec. 22, 2005.
Application No. 11/767,442 is a continuation-in-part of application No. 10/835,505, filed on Apr. 28, 2004, now Pat. No. 7,820,145.
Application No. 10/835,505 is a continuation-in-part of application No. 11/430,437, filed on May 9, 2006, now Pat. No. 7,645,803.
Application No. 11/767,442 is a continuation-in-part of application No. 11/430,599, filed on May 9, 2006, now Pat. No. 7,704,518.
Application No. 11/430,599 is a continuation-in-part of application No. 10/835,505, filed on Apr. 28, 2004, now Pat. No. 7,820,145.
(60) Provisional application No. 60/492,385, filed on Aug. 4, 2003.
(60) Provisional application No. 60/429,546, filed on Nov. 29, 2002.
(60) Provisional application No. 60/679,020, filed on May 9, 2005.
(60) Provisional application No. 60/784,793, filed on Mar. 21, 2006.
(60) Provisional application No. 60/492,385, filed on Aug. 4, 2003.
(60) Provisional application No. 60/530,015, filed on Dec. 16, 2003.
(60) Provisional application No. 60/679,020, filed on May 9, 2005.
(60) Provisional application No. 60/784,793, filed on Mar. 21, 2006.
(60) Provisional application No. 60/815,948, filed on Jun. 23, 2006.

(30) Foreign Application Priority Data
Oct. 25, 2002 (IL) 152486

(51) Int. Cl. A61K 31/554; A61K 31/505; A61K 47/10; A61K 31/4422

(52) U.S. Cl. CPC A61K 31/554; A61K 31/4422; A61K 31/505; A61K 47/10
USPC 514/211.03; 514/356; 514/275

(57) ABSTRACT

The present invention relates to a foamable therapeutic composition comprising: (a) a therapeutically effective concentration of at least one active agent selected from the group consisting of a channel agent, a cholinergic agent, and a nitric oxide donor; and (b) a foamable carrier comprising:
i. about 50% to about 98% of a solvent selected from the group consisting of water; a hydrophilic solvent; a hydrophobic solvent; a potent solvent; a polar solvent, a silicone, an emollient, and mixtures thereof;
ii. 0% to about 48% of a secondary solvent selected from the group consisting of water; a hydrophilic solvent; a hydrophobic solvent; a potent solvent; a polar solvent, a silicone, an emollient, a co-solvent, a penetration enhancer and mixtures thereof;
iii. a surface-active agent;
iv. about 0% to about 5% by weight of at least one polymeric agent; and
v. a liquefied or compressed gas propellant at a concentration of about 3% to about 25% by weight of the total composition;
wherein the composition is housed in a container and is substantially flowable, and
which upon release expands to form a breakable foam; and
wherein the foamable carrier is selected to generate a foam of good to excellent quality.

The invention further provides a method of treating, alleviating or preventing a disorder of mammalian subject, comprising administering such a composition to an afflicted target site.

### CROSS REFERENCE TO RELATED APPLICATIONS

This application claims the benefit of priority under 35 U.S.C. §119(e) to U.S. Patent Application No. 60/815,948, filed on Jun. 23, 2006, entitled “Foamable Compositions Comprising a Calcium Channel Blocker, a Cholinergic Agent and a Nitric Oxide Donor,” which is incorporated herein in its entirety.

This application is a continuation-in-part application of co-pending U.S. patent application Ser. No. 10/835,505, filed on Apr. 28, 2004, which claims the benefit of priority under 35 U.S.C. §119(e) to U.S. Patent Application No. 60/530,015, filed on Dec. 16, 2003, and U.S. Patent Application No. 60/492,385, filed on Aug. 4, 2003, all entitled “Oleaginous Pharmaceutical and Cosmetic Foam,” and all hereby incorporated in their entirety by reference.

This application is a continuation-in-part application of co-pending U.S. patent application Ser. No. 11/430,437, filed on May 9, 2006, entitled “Saccharide Foamable Compositions,” which claims the benefit under 35 U.S.C. §119(e) of U.S. Provisional Patent Application No. 60/679,020, filed on May 9, 2005, entitled “Hygroscopic Anti-Infective Compositions,” and both of which are hereby incorporated in their entirety by reference.

This application is a continuation-in-part application of co-pending U.S. patent application Ser. No. 11/430,599, filed on May 9, 2006, entitled “Foamable Vehicle and Pharmaceutical Compositions Thereof,” which claims the benefit under 35 U.S.C. §119(e) of U.S. Provisional Patent Application No. 60/784,793, filed on Mar. 21, 2006, entitled “Polyol Foamable Vehicle and Pharmaceutical Compositions Thereof,” both of which are herein incorporated by reference in its entirety.

This application is a continuation-in-part application of co-pending U.S. patent application Ser. No. 10/532,618, filed on Dec. 22, 2005, entitled “Cosmetic and Pharmaceutical Foam,” which is a 371 application of International Patent Application No. IB03/005527, designating the United States and filed on Oct. 24, 2003, which claims the benefit of priority under 35 U.S.C. §119(e) to U.S. Patent Application No. 60/492,546, filed on Nov. 29, 2002, both entitled “Cosmetic and Pharmaceutical Foam,” and which claims the benefit of priority under 35 USC§119(a) to Israeli Patent Application No. 152486, filed Oct. 25, 2002, all of which are hereby incorporated in their entirety by reference.

This application is a continuation-in-part of co-pending U.S. application Ser. No. 10/911,367, filed on Aug. 4, 2004, entitled “Foam Carrier Containing Amphiphilic Copolymeric Gelling Agent,” which claims the benefit of priority under 35 U.S.C. §119(e) to U.S. Patent Application No. 60/492,385, filed on Aug. 4, 2003, both entitled “Foam Carrier Containing Amphiphilic Copolymer Gelling Agent,” and both of which are hereby incorporated in their entirety by reference.

### BACKGROUND OF THE INVENTION

This invention relates to foamable pharmaceutical compositions.

External topical administration is an important route for the administration of drugs in disease treatment. Many groups of drugs, including, for example, antibiotic, anti-fungal, anti-inflammatory, anesthetic, analgesic, anti-allergic, corticosteroid, retinoid and anti-proliferative medications are preferably administered in hydrophobic media, namely ointment; or in semi-solid creams. However, ointments and creams often form an impermeable barrier, so that metabolic products and excreta from the wounds to which they are applied are not easily removed or drained away. Furthermore, it is difficult to administer creams and ointments onto damaged tissues, so the efficacy of the drug is reduced. In addition, ointments and creams often do not create an environment for promoting respiration of the wound tissue and it is not favorable to the normal respiration of the skin. Foam offers an alternative form of products for topical administration, which is more convenient to use and does not require rubbing in order to facilitate drug spreading and absorption.

Foams and, in particular, foams that are substantially based on non-aqueous solvents are complicated systems which do not form under all circumstances. U.S. patent application Ser. No. 10/835,505 entitled “Oleaginous Pharmaceutical and Cosmetic Foam” describes stable oleaginous cosmetic or therapeutic foam compositions containing certain active agents, having unique therapeutic properties and methods of treatment using such compositions. The foamable carrier includes at least one solvent selected from a hydrophobic solvent, a silicone oil, an emollient, a co-solvent, and mixtures thereof, wherein the solvent is present at a concentration of about 70% to about 96.5% by weight of the total composition, at least a non-ionic surface-active agent at a concentration of about 0.1% to less than about 10% by weight of the total composition; at least one gelling agent at a concentration of about 0.1% to about 5% by weight of the total composition; a therapeutically effective amount of at least one active agent; and at least one liquefied or compressed gas propellant, at a concentration of about 3% to about 25% by weight of the total composition.

Huggins et al. describes hydroalcoholic foam compositions in WO 2004/071479.

A fissure is a split in the skin of the distal anal canal. It is a common complaint in young adults with a roughly equal incidence in both sexes. Acute fissures are very common and most heal spontaneously, but a proportion progress to form a chronic linear ulcer in the anal canal and show great reluctance to heal without intervention. Treatment has remained largely unchanged for over 150 years and the pathogenesis of anal fissure is not fully understood. The passage of a hard stool bolus has traditionally been thought to cause anal fissure. Thus for acute fissures the avoidance of constipation, such as involving a high bran diet, has been used as treatment for many years.

Kamm et al. in US 2004/0028752 discloses topical pharmaceutical compositions comprising a cholinergic drug or a calcium channel blocker, whereby anal fissures, haemorrhoids and other benign anal disorders can be treated by local application to the anus of a cholinergic drug or a calcium channel blocker or a mixture thereof.

Calcium channel blockers are a group of drugs that inhibit the entry of calcium into cells or inhibit the mobilization of calcium from intracellular stores, resulting in slowing of atrioventricular and sinoatrial conduction and relaxation of arterial smooth and cardiac muscle. They are used in the treatment of angina, cardiac arrhythmias, and hypertension.

Topical pharmaceutical compositions comprising a cholinergic agent or a calcium channel blocker are disclosed in WO 98/36733. However, although it is mentioned therein that the compositions may be formulated in various forms, including foam, there is no guidance how to make a foam and the actual preparation of foams is not specifically taught or exemplified therein.

U.S. Pat. No. 6,455,076 discloses compositions for inhibiting skin irritations comprising a topical vehicle, an irritant ingredient; and an anti-irritant amount of at least about 10 mM of aqueous-soluble divalent tin cation in combination with another second agent, which can be a potassium channel blocker, a calcium channel blocker, or a sodium channel, to name just a few agents. This patent discloses a variety of suitable vehicles, including, inter alia, foams, but the preparation of foam is not taught or exemplified in the specification.

WO 01/54679 discloses a local, topical transdermal anesthetic and vasodilator formulation, wherein the topical vasodilator may be a calcium antagonist. Again, although several carrier forms are mentioned therein, the actual preparation of foams is not specifically taught or exemplified.

WO 03/075851 discloses compositions and methods for the treatment of anorectal disorders using NO donors, calcium channel blockers, cholinergic modulators etc. Again, although several carrier forms are mentioned therein, the actual preparation of foams is not specifically taught or exemplified.

U.S. Patent Application No. 2004/0038912 discloses compositions comprising, in a physiologically acceptable medium, at least one O-acyl product derived from glucose, and further comprising a pharmaceutical agent, such as a calcium antagonist. However, no foams are disclosed or exemplified therein.

U.S. Patent Application No. 2005/0054991 discloses a dosing device for topically administering a pharmaceutical formulation, wherein the therapeutic agents which can be used with the dosing system of the invention include all drugs which can be delivered on or through the skin for either a local or systemic effect, for example sodium or calcium channel blockers. This patent mentions a variety of suitable vehicles, including, inter alia, foams, but the preparation of foam is not taught or exemplified in the specification.

U.S. Patent Application No. 2005/0276836 discloses a vaginal device for delivering a therapeutical or health-enhancing agent, wherein this agent is formulated as a mucoadhesive composition and comprises at least one therapeutical agent which may be a calcium channel antagonist, or a potassium channel blocker. This patent discloses a variety of suitable vehicles, including, inter alia, foams, but the preparation of foam is not taught or exemplified in the specification with regard to calcium channel antagonists or potassium channel blockers.

European Journal of Pharmaceutics and Biopharmaceutics 46 (1998) 265-271, titled “Codiffusion of propylene glycol and dimethyl isosorbide in hairless mouse skin” reveals that Azone, polyethylene glycol 400, dimethyl isosorbide and propylene glycol, alone or in combinations, enhance the skin absorption of nifedipine. Dis Colon Rectum. 1996; 39(2):212-6, titled “Effect of nifedipine on rectoanal motility” and Dis Colon Rectum. 2000; 43(3):430-1, titled “Nifedipine for local use in conservative treatment of anal fissures: preliminary results of a multicenter study” suggest that nifedipine can be of some use in relieving symptoms in patients with hemorrhoids or anal fissure. Dis Colon Rectum. 2000; 43(10):1359-62, titled “Topical diltiazem and bethanechol decrease anal sphincter pressure and heal anal fissures without side effects” and Br J Surg. 2001; 88(4):553-6, titled “Topical diltiazem ointment in the treatment of chronic anal fissure” conclude that both topical diltiazem and bethanechol substantially reduce anal sphincter pressure and achieve fissure healing to a similar degree reported with topical nitrates, but without side effects. Dis Colon Rectum 2002; 45(11):1468-1475 describes the use of topical nifedipine with lidocaine for treatment of chronic anal fissure. DARU 2003; 11(1):19-22 describes the healing effect of topical nifedipine on skin wounds of diabetic rats. Dermatology Online Journal 11 (2):8 provides a review of uses of calcium antagonists in dermatology.

U.S. Patent Application No. 20020143188 discloses compositions comprising at least one potassium channel activator, and methods for treating or preventing sexual dysfunctions, cardiovascular disorders, cerebrovascular disorders, hypertension, asthma, baldness, urinary incontinence, epilepsy, sleep disorders, gastrointestinal disorders, migraines, irritable bowel syndrome and sensitive skin. This patent discloses a variety of suitable vehicles, including, inter alia, foams, but the preparation of foam is not taught or exemplified in the specification.

U.S. Pat. No. 6,562,355 discloses a cosmetic/dermatological composition suited for treating skin redness/edema and/or sensitive skin, comprising a synergistically effective amount of comixture of escin and dextran sulfate, a topically applicable, physiologically acceptable vehicle, diluent or carrier therefor, and optionally further comprising an NO-synthase inhibitor, a sodium-channel inhibitor, a potassium-channel opener etc. Foams are not disclosed therein.

U.S. Pat. No. 5,196,405 teaches a therapeutic method for alleviating symptoms of hemorrhoids by applying a hemorrhoidal composition comprising a therapeutic amount of sucralfate and a pharmaceutically-acceptable vehicle specially adapted for topical application, wherein the hemorrhoidal composition is administered in conjunction with a compound such as an anticholinergic. This patent discloses a variety of suitable vehicles, including, inter alia, foams, but the preparation of foam is not taught or exemplified in the specification.

U.S. Pat. No. 5,858,371 teaches a pharmaceutical composition for the treatment of anorectal and colonic diseases comprising a pharmaceutically acceptable base and an effective amount of a flavanoid containing extract from the plant Euphorbia pros, further comprising an additional therapeutic agent may be an anesthetic in combination with an anti-cholinergic agent. No foams are disclosed.

U.S. Patent Application No. 20030031693 discloses a cosmetic/dermatological composition suited for treating afflictions of the skin, comprising at least one hydroxystilbene compound, at least one ascorbic acid compound, and, inter alia, at least one calcium antagonist or potassium channel opener. No foams are disclosed.

U.S. Pat. No. 6,258,374 teaches a pharmaceutical composition for rectal or vaginal administration which comprises clotrimazole, as an exemplary fungicide, in a water-soluble collapsible foam structure, which is formed by mixing two separate substances within the composition, thereby producing a gas which contacts a polymer stabilizer. Foams generated by releasing the formulation from a pressurized canister are not disclosed, taught or exemplified. It is important to note that although clotrimazole is a potassium channel inhibitor, this aspect is neither taught nor suggested in the application.

U.S. Patent Application Nos. 2005/0255048 and 20070059253 disclose foamable delivery systems which include clotrimazole as an antifungal agent, in the treatment of a variety of skin conditions. These publications disclose dispersion of the formulations both through a propellant-pressurized container and in a hand-pumped or squeezed non-pressurized container. It is important to note that although clotrimazole is a potassium channel inhibitor, this aspect is neither taught nor suggested in the application.

WO 06/010589 discloses formulations adapted particularly for insertion of a pharmacologically active agent into a body cavity, and in one particular, the formulation is in a form of a gel at room temperature, which upon application, expands into a mousse or foam, wherein the compositions and formulations are desirably packaged as a metered aerosol, a single-shot vial, or as a two-compartmental system such as a “bag-in-can” type aerosol product. The pharmacologically active agent may be clotrimazole, used to treat fungal or bacterial infection.

WO 02/062324 discloses the use of a smooth muscle tone modulator in the manufacture of a medicament for use in the topical treatment of oesophageal motility disorders and gastro-oesophageal reflux disease, wherein the smooth muscle relaxant may be a calcium channel blocker (e.g. diltiazem or nifedipine), a potassium channel opener, a nitric oxide donor (e. g. glyceryl trinitrate, isosorbide trinitrate, L-arginine, S-nitroso-N-acetylpenicillamine or nitroprusside) etc. This patent discloses a formulation which is preferably in a swallowable form selected from solution, emulsion, gel or foam, but the preparation of foam is not taught or exemplified in the specification.

GB Patent No. 2166651 discloses controlled release powder containing discrete micro-particles for use in edible, pharmaceutical and other controlled release compositions, wherein the powder comprises particles containing an active ingredient, such as glibenclamid, a potassium channel blocker, and the particles of the powder have an average size in the range of 0.1 to 125 pm. This patent discloses a variety of suitable vehicles, including, inter alia, foams, but the preparation of foam is not taught or exemplified in the specification.

U.S. Patent Application No. 2003/0078172 discloses a foaming composition for topical application comprising: water, at least one wax, and a surfactant system, wherein the composition exhibits a paracrystalline phase at a temperature above 30° C. and below 45° C., further comprising, inter alia, clotrimazole. However, this publication relates to rinsable foamable creams, and foams generated by releasing the formulation from a pressurized canister are not disclosed, taught or exemplified.

U.S. Pat. No. 5,693,676 teaches a nitric oxide donor composition and method for treatment of anal disorders. Foams are not disclosed therein.

There remains an unmet need for improved, easy to use, stable and non-irritating foam formulations, intended for treatment of dermal and mucosal tissues, including treatment of anal disorders. Particularly, there remains an unmet need for improved, easy to use, stable and non-irritating anti-infective foam formulations, with unique therapeutic properties.

### SUMMARY OF THE INVENTION

This invention relates to foamable pharmaceutical compositions and methods of using such compositions as described in the appended claims. In one aspect, the present invention relates to a foamable therapeutic composition comprising a therapeutically effective concentration of at least one active agent selected from the group consisting of calcium channel blocker, a cholinergic agent and a nitric oxide donor; about 50% to about 98% of a polar solvent selected from the group consisting of (1) a polyol; and (2) a polyethylene glycol; 0% to about 48% of a secondary polar solvent; about 0.2% to about 5% by weight of a surface-active agent; about 0.01% to about 5% by weight of at least one polymeric agent; and a liquefied or compressed gas propellant at a concentration of about 3% to about 25% by weight of the total composition.

In one other aspect, the present invention further relates to a method of treating, alleviating or preventing a disorder of mammalian subject, comprising administering such compositions to an afflicted target site.

In one or more embodiments, the channel agent is selected from the group consisting of a calcium channel blocker, a potassium channel agent, a sodium channel agent and a chloride channel agent.

In one or more embodiments, the foamable carrier is selected from the group consisting of oil-in-water emulsions, water-in-oil emulsions, oleaginous formulations, hydrophilic solvent and water formulations; non aqueous or substantially non aqueous polyethylene glycol based compositions, non aqueous or substantially non aqueous propylene glycol based compositions, non aqueous or substantially non aqueous petrolatum and oil based compositions, and non aqueous or substantially non aqueous oil based compositions,

In one or more embodiments, one or more optional agents are selected from the group consisting of a co-emulsifier or foam stabilizer, a co polymeric agent, a viscosity, bulking or firming agent, a foam adjuvant, a co-solvent; a penetration enhancer, a stabilizer, a modulating agent, a drying agent, and an agent capable of having an occlusive effect.

In one or more embodiments, the calcium channel blocker is suitable for a biological activity selected from the group consisting of (i) vascular smooth muscle cell growth and/or proliferation, (ii) inhibition of growth and/or proliferation of fibroblasts, (iii) inhibition of the synthesis of extracellular matrix proteins, (iv) immunomodulation, (v) inhibition of mast cell degranulation and/or platelet aggregation, (vi) suppression of neutrophil adhesion, (vii) suppression of superoxide anion production, and (viii) analgesic effect.

In one or more embodiments, the calcium channel blocker is selected from the group consisting of an amlodipine, anipamil, barnidipine, benidipine, bepridil, darodipine, diltiazem, efonidipine, felodipine, isradipine, lacidipine, lercanidipine, lidoflazine, manidipine, mepirodipine, nicardipine, nifedipine, niludipine, nilvadipine, nimodipine, nisoldipine, nitrendipine, perhexiline, tiapamil, verapamil, and pharmaceutically acceptable salts and derivatives thereof.

In one or more embodiments, the cholinergic drug is selected from the group consisting of acetylcholine, bethanechol, carbachol, methacholine, pilocarpine, an anticholinesterase of ambenonium, neostigmine, physostigmine, pyridostigmine, dyflos, ecothinopate, and pharmaceutically acceptable salts thereof.

In one or more embodiments, the nitric oxide donor is selected from the group consisting of an inorganic nitrite, an inorganic nitrate, an organic nitrite, an organic nitrate, a nitrite ester of a polyol, a nitrate ester of a polyol molsidomine and its metabolites, a diazeniumdiolate, a S-nitrosothiol, an iron-sulphur nitrosyl, sodium nitrite, ethylene glycol dinitrate, isopropyl nitrate, amyl nitrite, amyl nitrate, ethyl nitrite, butyl nitrite, isobutyl nitrite, octyl nitrite, glyceryl-1-mononitrate, glyceryl-1,2-dinitrate, glyceryl-1,3-dinitrate, nitroglycerin, butane-1,2,4-triol-trinitrate, erythrityl tetranitrate, pentaerythrityl tetranitrate, sodium nitroprusside, clonitrate, erythrityl tetranitrate, isosorbide mononitrate, isosorbide dinitrate, mannitol hexanitrate, mesoionic oxatriazole, pentaerythritol tetranitrate, penetrinitol, triethanolamine trinitrate, trolnitrate phosphate (triethanolamine trinitrate diphosphate), propatylnitrate, nitrite esters of sugars, nitrate esters of sugars, sodium nitroprusside, nicorandil, apresoline, diazoxide, hydralazine, hydrochlorothiazide, minoxidil, pentaerythritol, tolazoline, scoparone (6,7-dimethoxycoumarin) sinitrodil, sildenafil, vardenafil, tadalafil, 4-Ethyl-2-[(Z)-hydroxyiminol]-5-nitro-3(E)-hexeneamide, and pharmaceutically acceptable salts derivatives and isomers thereof.

In one or more embodiments, the potassium channel agent is selected from the group consisting of a potassium channel opener, a potassium channel modulator and a potassium channel blocker.

In one or more embodiments, the potassium channel opener is selected from the group consisting of doxefazepam, brotizolam, lorazepam, haloxazolam, nimetazepam, cinolazepam, clonazepam, flunitrazepam, midazolam, nicorandil, pinacidil, lormetazepam, flupirtine, minoxidil, ibutilide fumarate, loprazolam, rilmazafone, estazolam, temazepam and any potassium channel openers listed in patent WO04093895A1;
wherein the potassium channel modulator is selected from the group consisting of a dendrotoxin, dendrotoxin I, dendrotoxin K, alpha-dendrotoxin, beta-dendrotoxin, gamma-dendrotoxin, margatoxin, stichodactyla toxin, tityustoxin K, apamin, charylotoxin, clotrimazole, dequalinium chloride, iberiotoxin, kaliotoxin, minoxidil, neuropeptide Y, noxiustoxin, tolbutamide, chlorpropamide, glibenclamide, glipizide, nategliniide, repagliniide, glyburide, tolazamide, nicorandil, fampridine and penitrem A or a pharmaceutically acceptable salt or prodrug thereof; and
wherein the potassium channel blocker is selected from includes 4-aminopyridine, almikalant, ambasilide, amiodarone, apamin, azimilide, charybdotoxin, clofilium, clotrimazole, correolide, dequalinium chloride, dofetilide, glibenclamide, glyburide, ibutilide, paxilline, procain, sematilide, sotalol, tedisamil, tetramethylammonium and tolazamide.

In one or more embodiments, the sodium channel agent is selected from the group consisting of local anesthetics such as lidocaine; anticonvulsants such as phenytoin and carbamazepine, antiarrhythmics such as mexiletine; akaloid based toxins such as veratridine; batrachotoxin and aconitine; Diterpene based toxins such as grayanotoxin; and peptide based toxins such as μ-conotoxin; δ-atracotoxin.

In one or more embodiments, the chloride channel agent is a chloride channel blocker/opener used in the treatment of Bartter's syndrome, Dent's Diseasel, and Thomsen disease.

In one or more embodiments, the breakable foam comprises liquid crystals.

In one or more embodiments, the liquid crystals comprise four, five, six or seven sided structures forming an interconnecting matrix.

In one or more embodiments, the interconnecting matrix forms triangular like Y shaped connections.

In one or more embodiments, the breakable foam comprises micro or nano particles, crystals or bodies.

In one or more embodiments, the formulation up to 85% of water, or up to 25% of water, or the carrier is substantially water-free.

In one or more embodiments, the carrier is substantially alcohol-free.

The foamable therapeutic composition in one or more embodiments, is substantially resistant to one or more Freeze-Thaw cycles (FTC).

In one or more embodiments, the polar solvent is selected from the group consisting of a polyol and a polyethylene glycol (PEG), wherein the polyol is selected from the group consisting of a diol and a triol.

In one or more embodiments, the diol is selected from the group consisting of propylene glycol, butanediol, butenediol, butynediol, pentanediol, hexanediol, octanediol, neopentyl glycol, 2-methyl-1,3-propanediol, diethylene glycol, triethylene glycol, tetraethylene glycol, dipropylene glycol and dibutylene glycol and wherein the triol is selected from the group consisting of glycerin, butane-1,2,3-triol, butane-1,2,4-triol and hexane-1,2,6-triol.

In one or more embodiments, the polyol comprises at least one diol and at least one triol, and wherein the ratio between the diol and triol is between about 9:1 and about 1:1.

In one or more embodiments, the PEG is selected from the group consisting of PEG 200, PEG 300, PEG 400, PEG 600, PEG 1000, PEG 4000, PEG 6000 and PEG 8000.

In one or more embodiments, the carrier composition comprises a mixture of at least one polyol and at least one PEG.

In one or more embodiments, the secondary solvent is a polar solvent selected from the group consisting of dimethyl isosorbide, tetrahydrofurfuryl alcohol, polyethyleneglycol, ether, DMSO, a pyrrolidone, N-Methyl-2-pyrrolidone, 1-Methyl-2-pyrrolidinone, ethyl proxitol, dimethylacetamide, a PEG-type surfactant, an alpha hydroxy acid, lactic acid and glycolic acid.

In one or more embodiments, the composition comprises (1) at least one polar solvent selected from a diol, a triol and PEG, and (2) at least one secondary polar solvent.

The foamable composition of claim 1, wherein the polymeric agent is selected from the group consisting of locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, an amine-bearing polymer, chitosan, alginic acid, hyaluronic acid, a chemically modified starch, a carboxyvinyl polymer, polyvinylpyrrolidone, polyvinyl alcohol, a polyacrylic acid polymer, a polymethacrylic acid polymer, polyvinyl acetate, a polyvinyl chloride polymer, a polyvinylidene chloride polymer, methylcellulose, hydroxypropyl cellulose, hydroxypropyl methylcellulose, hydroxyethyl cellulose, hydroxy propylmethyl cellulose, methylhydroxyethylcellulose, methyl hydroxypropylcellulose, hydroxyethylcarboxymethylcellulose, carboxymethyl cellulose, carboxymethylcellulose carboxymethylhydroxyethylcellulose, a cationic cellulose, PEG 1000, PEG 4000, PEG 6000 and PEG 8000.

In one or more embodiments, the polymeric agent is dispersible in the polyol or in the mixture of a polyol and a secondary polar solvent.

In one or more embodiments, the polymeric agent is selected from the group consisting of Carbopol® 934, Carbopol® 940, Carbopo® 941, Carbopol® 980, Carbopol® 981, hydroxypropylcellulose and carbomer.

In one or more embodiments, the surface-active agent has a HLB value between about 2 and about 9 or is a combination of two or more surface active agents having a mean HLB value between about 2 and about 9.

In one or more embodiments, the surface-active agent has a HLB value between about 7 and about 14 or is a combination of two or more surface active agents having a mean HLB value between about 7 and about 14.

In one or more embodiments, the surface-active agent has a HLB value between about 9 and about 19 or is a combination of two or more surface active agents having a mean HLB value between about 9 and about 19.

In one or more embodiments, the surface-active agent is a solid, a liquid or a mixture thereof.

In one or more embodiments, the surface active agent is selected from the group consisting of a polysorbate, polyoxyethylene (20) sorbitan monostearate, polyoxyethylene (20) sorbitan monooleate, a polyoxyethylene fatty acid ester, Myrj 45, Myrj 49, Myrj 52 and Myrj 59; a polyoxyethylene alkylyl ether, polyoxyethylene cetyl ether, polyoxyethylene palmityl ether, polyethylene oxide hexadecyl ether, polyethylene glycol cetyl ether, brij 38, brij 52, brij 56 and brij W1, a sucrose ester, a partial ester of sorbitol, sorbitan monolaurate, sorbitan monolaurate a monoglyceride, a diglyceride, isoceteth-20 and a sucrose ester.

The foamable carrier of claim 1 wherein the surface active agent is selected from the group consisting of steareth 2, glyceryl monostearate/PEG 100 stearate, Glyceryl Stearate, Steareth-21, peg 40 stearate, polysorbate 60, polysorbate 80, sorbitan stearate, aureth 4, Sorbitan monooleate, ceteareth 20, steareth 20, ceteth 20, Macrogol Cetostearyl Ether, ceteth 2, PEG-30 Dipolyhydroxystearate, sucrose distearate, polyoxyethylene (100) stearate, PEG 100 stearate, laureth 4, cetomacrogol ether, Cetearyl alcohol, Cetearyl glucoside, Oleyl alcohol, Steareth-2, Diisopropyl adipate, Capric/caprilic triglicerides, and mixtures thereof.

In one or more embodiments, the surface-active agent comprises a non-ionic surface-active agent.

In one or more embodiments, the surface-active agent further comprises an ionic surfactant selected from the group consisting of a cationic surfactant, a zwitterionic surfactant, an amphoteric surfactant and an ampholytic surfactant.

In one or more embodiments, the surface-active agent comprises a mixture of at least one non-ionic surfactant and at least one ionic surfactant in a ratio selected from

about 100:1 to about 6:1; and

about 1:1 to about 20:1.

In one or more embodiments, the hydrophobic solvent is selected from the group consisting of mineral oil, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, unsaturated or polyunsaturated oils, olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils; essential oils, silicone oils, dimethicone, cyclomethicone, polyalkyl siloxane, polyaryl siloxane, polyalkylaryl siloxane, a polyether siloxane copolymer and a poly(dimethylsiloxane)-(diphenyl-siloxane) copolymer.

In one or more embodiments, a foam adjuvant is selected from the group consisting of a fatty alcohol, a fatty acid and a hydroxyl fatty acid.

In one or more embodiments, the foam includes an additional active agent.

In one or more embodiments, the additional active agent is selected from the group consisting of active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, anti-yeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, vitamins, vitamin D derivatives, wound healing agents and wart removers.

In one or more embodiments, the active agent is unstable in the presence of water.

In one or more embodiments, the composition is formulated for slow release.

In another aspect, a method of treating, alleviating or preventing a dermatological reaction, sensation or disorder of a mammalian subject, includes administering an effective amount of a therapeutic composition to a target site on a mammalian subject cpmprising a therapeutically effective concentration of at least one active agent selected from the group consisting of a channel agent; a cholinergic agent; a nitric oxide donor, or a related agent, wherein the channel agent is selected from the group consisting of a calcium channel blocker, a potassium channel agent; a sodium channel agent and a chloride channel agent;

wherein the foamable carrier composition comprising an active agent is selected from the group consisting of:
an aqueous non alcoholic foamable carrier comprising about 3% to about 80% propylene glycol; a surfactant; about 0.1% to about 5% of a polymeric agent; water and about 3% to about 25% of a propellant;
an oleaginous non alcoholic foamable carrier comprising about 3% to about 80% oil, oil like substance or petrolatum; a surfactant; about 0.1% to about 5% of a polymeric agent; water and about 3% to about 25% of a propellant;
an oil in water non alcoholic foamable carrier comprising about 3% to about 98% water; a surfactant; about 0.1% to about 5% of a polymeric agent; an oil or oil like substance and about 3% to about 25% of a propellant;
a water in oil non alcoholic foamable carrier comprising about 3% to about 70% water; a surfactant; about 0.1% to about 5% of a polymeric agent; an oil or oil like substance and about 3% to about 25% of a propellant;
a non aqueous non alcoholic foamable carrier comprising about 3% to about 98% propylene glycol; a surfactant; about 0.1% to about 5% of a polymeric agent; and about 3% to about 25% of a propellant;
a non aqueous non alcoholic foamable carrier comprising about 3% to about 98% PEG; a surfactant; about 0% to about 5% of a polymeric agent; and about 3% to about 25% of a propellant;
a non aqueous non alcoholic foamable carrier comprising about 3% to about 98% mixture of petroleum and oil or oil like substance; a surfactant; about 0% to about 5% of a polymeric agent; and about 3% to about 25% of a propellant;
a non aqueous non alcoholic foamable carrier comprising about 3% to about 98% mixture of oil or oil like substance; a silicone; a surfactant; about 0% to about 5% of a polymeric agent; and about 3% to about 25% of a propellant;
an oil in water high oil content DIPA formulation comprising: about 20% to about 40% oil phase; about 2% to about 5% surfactants; about 1% to about 2% foam adjuvants; about 0% to about 0.7% polymers; about 5% to about 30% hydrophilic solvent; water and about 3% to about 25% propellant;
an oil in water non comedogenic formulation comprising: about 2% to about 30% of oil phase, about 1% to about 5% surfactant, about 0.2% to 1.5% stabilizing polymer, about 20% to about 60% of an hydrophilic solvent such as PEG400; water and about 3% to about 25% propellant;
a hydrophilic solvent and water single phase formulation comprising: homogeneous mixture of about 30% to about 70% hydrophilic solvents; alcohols and polyols (PEG200, PEG400, ethanol, Propylene glycol), and water; about 1% to about 5% stabilizing surfactants, about 0.5% to about 2.0% polymer, and about 3% to about 25% propellant;

wherein the presence of significant amounts of an active agent in a composition does not prevent a foam of good or satisfactory quality from being produced and wherein the composition is stored in an aerosol container is flowable and upon release expands to form a breakable foam which is spread at, about and within the target site when mechanical shear force is applied to said breakable foam.

In one or more embodiments, the target site is selected from the group consisting of the skin, a body cavity, a mucosal surface, the nose, the mouth, the eye, the ear canal, the respiratory system, the vagina, the rectum, the anus, the anal canal and the internal anal sphincter.

In one or more embodiments, the disorder is selected from the group consisting of a benign anal disorder, an anal fissure and a haemorroidal condition, keloids, hypertrophic scars, wound, ulcer and burn, sexual dysfunction, and post-surgical adhesions.

In one or more embodiments, the disorder is a systemic disorder, that responds to treatment with a calcium channel blocker, a potassium channel opener, a cholinergic agent, or a nitric oxide donor; wherein the method comprises transdermal or trans-mucosal delivery of a calcium channel blocker, a potassium channel opener, a cholinergic agent, or a nitric oxide donor.

In one or more embodiments, the disorder is a dermatological disorder selected from the group consisting of dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, ecthyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, grannuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo.

The method of claim 66, wherein the disorder is selected from the group consisting of chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, and polyps of the colon and rectum.

In one or more embodiments, the calcium channel blocker is selected from the group consisting of an amlodipine, anipamil, barnidipine, benidipine, bepridil, darodipine, diltiazem, efonidipine, felodipine, isradipine, lacidipine, lercanidipine, lidoflazine, manidipine, mepirodipine, nicardipine, nifedipine, niludipine, nilvadipine, nimodipine, nisoldipine, nitrendipine, perhexiline, tiapamil, verapamil, and pharmaceutically acceptable salts and derivatives thereof.

In one or more embodiments, wherein the cholinergic drug is selected from the group consisting of acetylcholine, bethanechol, carbachol, methacholine, and pilocarpine, or an anticholinesterase of ambenonium, neostigmine, physostigmine, pyridostigmine, dyflos, and ecothinopate, and pharmaceutically acceptable salts of thereof.

In one or more embodiments, the nitric oxide donor is selected from the group consisting of an inorganic nitrite, an inorganic nitrate, an organic nitrite, an organic nitrate, a nitrite ester of a polyol, a nitrate ester of a polyol molsidomine and its metabolites, a diazeniumdiolate, a S-nitrosothiol, an iron-sulphur nitrosyl, and pharmaceutically acceptable salts, sodium nitrite, ethylene glycol dinitrate; isopropyl nitrate; amyl nitrite, amyl nitrate, ethyl nitrite, butyl nitrite, isobutyl nitrite, octyl nitrite, glyceryl-1-mononitrate, glyceryl-1,2-dinitrate, glyceryl-1,3-dinitrate, nitroglycerin, butane-1,2,4-triol-trinitrate; erythrityl tetranitrate; pentaerythrityl tetranitrate; sodium nitroprusside, clonitrate, erythrityl tetranitrate, isosorbide mononitrate, isosorbide dinitrate, mannitol hexanitrate, mesoionic oxatriazole, pentaerythritol tetranitrate, penetrinitol, triethanolamine trinitrate, trolnitrate phosphate (triethanolamine trinitrate diphosphate), propatylnitrate, nitrite esters of sugars, nitrate esters of sugars, sodium nitroprusside, nicorandil, apresoline, diazoxide, hydralazine, hydrochlorothiazide, minoxidil, pentaerythritol, tolazoline, scoparone (6,7-dimethoxycoumarin) sinitrodil, sildenafil, vardenafil, tadalafil, 4-Ethyl-2-[(Z)-hydroxyiminol]-5-nitro-3(E)-hexeneamide and pharmaceutically acceptable salts, isomers, analogs and derivatives thereof.

In one or more embodiments, the solvent comprises a mixture of diiopropyl adipate, capric/caprilic triglycerides and diethyl sebacate.

In one or more embodiment, the solvent comprises a single phase of water and a hydrophilic solvent.

In one or more embodiments, the foam further includes up to 25% of water.

In one or more embodiments, the carrier is substantially water-free.

In one or more embodiments, the carrier is substantially alcohol-free.

In one or more embodiments, the secondary solvent is a polar solvent is selected from the group consisting of dimethyl isosorbide, tetrahydrofurfuryl alcohol polyethyleneglycol, ether, DMSO, a pyrrolidone, N-Methyl-2-pyrrolidone, 1-Methyl-2-pyrrolidinone, ethyl proxitol, dimethylacetamide, a PEG-type surfactant, an alpha hydroxy acid, lactic acid and glycolic acid.

In one or more embodiments, the composition further contains a hydrophobic solvent.

In one or more embodiments, the composition further contains a foam adjuvant selected from the group consisting of a fatty alcohol, a fatty acid and a hydroxyl fatty acid.

In one or more embodiments, the composition further contains an additional active agent.

In one or more embodiments, the additional active agent is selected from the group consisting of active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, anti-yeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, vitamins, vitamin D derivatives, wound healing agents and wart removers.

In one or more embodiments, a foamable therapeutic composition comprises Minoxidil and an aqueous non alcoholic foamable carrier comprising about 3% to about 80% propylene glycol; a surfactant; about 0.1% to about 5% of a polymeric agent; water and about 3% to about 25% of a propellant.

In one or more embodiments, Minoxidil is about 5%.

In one or more embodiments, surfactant is a polysorbate.

In one or more embodiments, the foamable carrier is selected to create an effective delivery to the skin or mucosal layer whilst minimizing systemic penetration.

In one or more embodiments, the foamable carrier is selected to create a substantially flowable composition without the active ingredient precipitating out of solution.

In another aspect, a foamable therapeutic composition comprises Minoxidil and an aqueous non alcoholic foamable carrier comprising about 3% to about 80% propylene glycol; a surfactant; about 0.1% to about 5% of a polymeric agent; about 5% to about 20% of at least one pharmaceutically acceptable acid; water and about 3% to about 25% of a propellant.

In one or more embodiments, a pharmaceutically acceptable base is such that the pH of the composition is between about 4.5 to about 5.0.

In one or more embodiments, Minoxidil is from about 5% to about 20%.

In one or more embodiments, the acid is selected from the group consisting of lactic acid, stearic acid and citric acid.

In one or more embodiments, the surfactant is a polysorbate.

In one or more embodiments, the foamable carrier is selected to create an effective delivery to the skin or mucosal layer whilst minimizing systemic penetration.

In one or more embodiments, the foamable carrier is selected to create a substantially flowable composition without the active ingredient precipitating out of solution.

In another aspect, a foamable therapeutic composition comprises Minoxidil and an aqueous non alcoholic foamable carrier comprising: a surfactant; about 0.1% to about 5% of a polymeric agent; about 5% to about 20% of at least one pharmaceutically acceptable acid; about 50% to about 80% of water and about 3% to about 25% of a propellant.

In one or more embodiments, the composition further includes a pharmaceutically acceptable base such that the pH of the composition is between about 4.5 to about 5.0.

In one or more embodiments, the acid is selected from the group consisting of lactic acid, stearic acid and citric acid.

In one or more embodiments, the foamable carrier is selected to create an effective delivery to the skin or mucosal layer whilst minimizing systemic penetration.

In one or more embodiments, the foamable carrier is selected to create a substantially flowable composition without the active ingredient precipitating out of solution.

A foamable therapeutic composition may include Minoxidil and a waterless non alcoholic foamable carrier comprising about 3% to about 98% propylene glycol; a surfactant; about 0.1% to about 5% of a polymeric agent; and about 3% to about 25% of a propellant.

In one or more embodiments, the surfactant is a polysorbate.

In one or more embodiments, Minoxidil is about 5%.

In one or more embodiments, in the foamable carrier is selected to create an effective delivery to the skin or mucosal layer whilst minimizing systemic penetration.

In one or more embodiments, the foamable carrier is selected to create a substantially flowable composition without the active ingredient precipitating out of solution.

In another aspect, a method of treating hair loss disorders includes administering to a subject in need of such treatment a therapeutically effective amount of a foamable composition as described herein.

In one or more embodiments, the minoxidil is substantially targeted to the area of the hair follicles.

In one or more embodiments, the foamable carrier is selected to create an effective delivery to the skin or mucosal layer whilst minimizing systemic penetration

In one or more embodiments, the foamable carrier is selected to create a substantially flowable composition without the active ingredient precipitating out of solution.

In one or more embodiments, the foamable carrier is selected to create an effective delivery to the skin or mucosal layer whilst minimizing systemic penetration.

In one or more embodiments, the foamable carrier is selected to create a substantially flowable composition without the active ingredient precipitating out of solution.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

FIG. 1 is a microscopic photograph of foam Formulation MFP-010 containing 20% minoxidil disclosing no crystals.

FIG. 2 is a microscopic photograph of foam Formulation MFP-003 containing 25% minoxidil disclosing crystals after shear forces.

FIGS. 3.1-3.6 are microscopic photographs of Formulation 6 comprising 5% Minoxidil and disclosing that the foam comprises liquid crystals which disappear after being sujec to shear force; no regular minoxidil crystals are observed.

FIG. 4 is a microscopic photograph of Formulation M08(2) comprising 5% Minoxidil and disclosing no crystals.

FIG. 5 is a microscopic photograph of minoxidil in the region of the hair follicle after a formulation MNX008 containing minoxidil being applied to the hair.

FIGS. 6 and 7 are microscopic photographs of non aqueous Formulations M01 and M02; no minoxidil crystals are seen.

### DETAILED DESCRIPTION OF THE INVENTION

In one aspect, this invention relates to a therapeutic foam including at least one active agent selected from the group consisting of a channel agent; a cholinergic agent; a nitric oxide donor, or a related agent, wherein the channel agent is selected from the group consisting of a calcium channel blocker, a potassium channel agent; a sodium channel agent and a chloride channel agent; In one or more embodiments the therapeutic foam is a penetrating enhancing foam. In another aspect, the invention further relates to the use of the penetration enhancing foam. Non limiting examples are for the treatment of benign anal diseases where there is an associated anal sphincter spasm, particularly for the treatment of anal fissures and hemorrhoids, as well as wound, burn and scar conditions.

Non limiting examples are:

where the disorder is a benign anal disorder;

where the disorder is for the treatment of benign anal diseases where there is an associated anal sphincter spasm, particularly for the treatment of anal fissures and hemorrhoids, as well as wound, burn and scar conditions;

where the disorder is selected from the group consisting of an anal fissure and a haemorroidal condition, keloids, hypertrophic scars, wound, ulcer and burn;

where the disorder is a systemic disorder, that responds to treatment with a calcium channel blocker, a potassium channel opener, a cholinergic agent, or a nitric oxide donor; wherein the method comprises transdermal or trans-mucosal delivery of a calcium channel blocker, a potassium channel opener, a cholinergic agent, or a nitric oxide donor;

where the disorder consists of sexual dysfunction;

where the disorder is a dermatological disorder selected from the group consisting of dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, ecthyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, grannuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo;

where the disorder is selected from the group consisting of chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, and polyps of the colon and rectum;

where the disorder consists of post-surgical adhesions.

where the calcium channel blocker is suitable for a biological activity selected from the group consisting of (i) vascular smooth muscle cell growth and/or proliferation, (ii) inhibition of growth and/or proliferation of fibroblasts, (iii) inhibition of the synthesis of extracellular matrix proteins, (iv) immunomodulation, (v) inhibition of mast cell degranulation and/or platelet aggregation, (vi) suppression of neutrophil adhesion, (vii) suppression of superoxide anion production, and (viii) analgesic effect;

where the potassium channel agent is suitable for; treatment of hypertension; AHR; to reduce the affinity of anti-diabetic drugs towards the potassium channel; to reduce insulin release; to treat androgenetic alopecia (AA); to treat congestive heart failure, to treat penile erection disorder; to prevent premature labour; as anti-inflammatory agents; to decrease in responsiveness to excitatory stimuli; to cause smooth muscle relaxation & decrease blood pressure; and to ameliorate inflammation induced tissue damage.

where the sodium channel agent is suitable as local anesthetics; anticonvulsants

where the chloride channel agent is used in the treatment of Bartter's syndrome, Dent's Diseasel, and Thomsen disease.

According to one or more embodiments of the present invention, the foamable therapeutic composition includes:
a. a therapeutically effective concentration of at least one active agent selected from the group consisting of a channel agent, a cholinergic agent, and a nitric oxide donor; and
b. a foamable carrier comprising:
   a. about 50% to about 98% of a solvent selected from the group consisting of water; a hydrophilic solvent; a hydrophobic solvent; a potent solvent; a polar solvent, a silicone, an emollient, and mixtures thereof;
   b. 0% to about 48% of a secondary solvent selected from the group consisting of water; a hydrophilic solvent; a hydrophobic solvent; a potent solvent; a polar solvent, a silicone, an emollient, a co-solvent, a penetration enhancer and mixtures thereof;
   c. a surface-active agent;
   d. about 0% to about 5% by weight of at least one polymeric agent; and
   e. a liquefied or compressed gas propellant at a concentration of about 3% to about 25% by weight of the total composition;
wherein the composition is housed in a container and is substantially flowable and which upon release expands to form a breakable foam; and
wherein the foamable carrier is selected to generate a foam of good to excellent quality.

All % values are provided on a weight (w/w) basis.

Water, up to 25% of the composition, and more preferably up to 10%, and optional ingredients are added to complete the total mass to 100%.

Upon release from an aerosol container, the foamable carrier forms an expanded foam suitable for the treatment of an infected surface and for topical administration to the skin, a body surface, a body cavity or a mucosal surface.

In certain cases, the composition contains two active agents that require different pH environments in order to remain stable. For example, corticosteroids are typically stable at acidic pH (they have a maximum stability at a pH of about 4-6) and vitamin D analogues are typically stable at basic pH (they have a maximum stability at pH values above about 8).

In other cases, the active agent degrades in the presence of water, and therefore, in such cases the present of water in the composition is not desirable. Thus, in certain preferred embodiments, the composition is substantially non-aqueous.

The foamable compositions provide several features that are useful in topical foam applications.

The foamable carrier can create an effectively stable environment for the active agent by omitting ingredients which may destabilize the active agent and or adding ingredients which may stabilize the active ingredient.

The foamable carrier can create a substantially physically stable foamable composition for the active agent

The foamable carrier can create an effective delivery to the skin or mucosal layer whilst minimizing systemic penetration.

The foamable carrier can create a substantially flowable composition without the active ingredient precipitating out of solution.

### Channel Agents; Cholinergic Agents; Nitric Oxide Agents and Related Agents

### Ion Channels

Ion channels are pore-forming proteins that help to establish and control the small voltage gradient across the plasma membrane of all living cells by allowing the flow of ions down their electrochemical gradient. They are present in the membranes that surround biological cells.

### Calcium Channel Blockers

Calcium channel blockers are a chemically and pharmacologically heterogeneous group of drugs, but physiologically they all share the ability to selectively antagonize the calcium ion movements that are responsible for the excitation-contraction coupling in the cardiovascular system. Beyond their cardiovascular effects, calcium channel blockers are known to possess other effects, such as inhibition of the growth and proliferation of vascular smooth muscle cells and fibroblasts, inhibition of the synthesis of extracellular matrix proteins, immunomodulation, inhibition of mast cell degranulation and platelet aggregation and suppression of neutrophil adhesion and superoxide anion (O-2) production. Some calcium channel blockers also have analgesic effects.

Current therapeutic uses of calcium channel blockers include (but are not limited to) hypertension, angina, arrhythmia and subarachnoid hemorrhage. Calcium channel blockers may further relieve or prevent reactive vasodilation of migraine sufferers by inhibiting the vasoconstriction during the prodromal phase.

There are two main classes of calcium channel blockers: dihydropyridines (e.g., nifedipine, nicardipine, amlodipine, felodipine and nimodipine) and nondihydropyridines which include diltiazem (a benzothiazepine) and verapamil (a phenylalkylamine). Flunarizine is an antihistamine with calcium channel blocking activity.

In an embodiment of the present invention, the calcium channel blocker can be selected from the group consisting of an amlodipine, anipamil, barnidipine, benidipine, bepridil, darodipine, diltiazem, efonidipine, felodipine, isradipine, lacidipine, lercanidipine, lidoflazine, manidipine, mepirodipine, nicardipine, nifedipine, niludipine, nilvadipine, nimodipine, nisoldipine, nitrendipine, perhexiline, tiapamil, verapamil, pharmaceutically acceptable salts, isomers, analogs and derivatives thereof.

### Potassium Channel Agents

Potassium channel openers (KCOs) act by stimulating ion flux through a distinct class of potassium channels, and are thus used in a variety of medicinal applications [Potassium Channel Openers: Novel Therapeutics Approaches, by: Hagappa et al., in http://www.pharmabiz.com/article/ published on Wednesday, Nov. 20, 2002). For example, The phenomenon termed bronchia (or airway) hyperactivity (AHR) which contributes to the airway obstruction characteristic of asthma is related to an increased excitability of smooth muscle cells and/or the nervous elements of the airways. By increasing the efflux of potassium from these cells, KCOs would induce hyperpolarization and a decrease in responsiveness to excitatory stimuli

Potassium channel openers are also a new class of cardiovascular drugs, and are further used for the treatment of hypertension: after activation of KATP channel, potassium efflux occurs from the vascular cell membrane, which leads to hyperpolarization & results smooth muscle relaxation & decrease blood pressure.

KCOs can also be used to reduce the affinity of anti-diabetic drugs towards the potassium channel. Thus, pancreatic B-cell contraction is reduced and insulin release is inhibited. Another important use of KCOs is in the treatment of androgenetic alopecia (AA).

Other applications of KCOs, which are still under clinical developments are the treatment of congestive heart failure, the treatment of penile erection disorder and the prevention of premature labour.

Potassium channel modulators can for example act as anti-inflammatory agents

Destruction of self-tissue by unwanted inflammation is central to the pathology of numerous diseases and disorders. The inflammatory response consists of a series of events, involving several cell types and biological signalling molecules. Potassium (K⁺) channels are also involved in this process and may be potential therapeutic targets. The K_{ATP}, Kv1.3 and BKCa channels affect diverse aspects of inflammation and its associated disorders. K_{ATP} channels protect tissue against ischaemia/reperfusion (I/R) injury, Kv1.3 channels regulate T-cell activation and BKCa channels play a role in controlling cellular activities triggered by depolarisation. K_{ATP} and BKCa channel openers and Kv1.3 channel blockers have all been shown experimentally to exert anti-inflammatory effects. K⁺ channel modulators may also be helpful to ameliorate inflammation induced tissue damage.

A non limiting list of potassium ion channel modulators includes dendrotoxin, dendrotoxin I, dendrotoxin K, alpha-dendrotoxin, beta-dendrotoxin, gamma-dendrotoxin, margatoxin, stichodactyla toxin, tityustoxin K, apamin, charylotoxin, clotrimazole, dequalinium chloride, iberiotoxin, kaliotoxin, minoxidil, neuropeptide Y, noxiustoxin, tolbutamide, chlorpropamide, glibenclamide, glipizide, nategliniide, repagliniide, glyburide, tolazamide, nicorandil, fampridine and penitrem A, or a pharmaceutically acceptable salt or prodrug thereof.

A non limiting list of potassium ion channel openers includes doxefazepam, brotizolam, lorazepam, haloxazolam, nimetazepam, cinolazepam, clonazepam, flunitrazepam, midazolam, nicorandil, pinacidil, lormetazepam, flupirtine, minoxidil, ibutilide fumarate, loprazolam, rilmazafone, estazolam, temazepam. A further list of potassium channel openers can be found in patent WO04093895A1: p. 146 onwards and which are incorporated herein by reference and may also be deliverable by the foamable carriers of the present invention.

A non limiting list of potassium channel blockers includes 4-aminopyridine, almikalant, ambasilide, amiodarone, apamin, azimilide, charybdotoxin, clofilium, clotrimazole, correolide, dequalinium chloride, dofetilide, glibenclamide, glyburide, ibutilide, paxilline, procain, sematilide, sotalol, tedisamil, tetramethylammonium and tolazamide.

### Sodium Channel Agents

Sodium channels (also known as “voltage-gated sodium channels”) are integral membrane proteins that conduct sodium ions (Na+) through a cell's plasma membrane. Many of the ionotropic receptors are also able to conduct sodium ions and are discussed elsewhere. In excitable cells such as neurons and myocytes, sodium channels are responsible for the rising phase of action potentials.

The pore of sodium channels contains a selectivity filter made of negatively charged amino acid residues, which attract the positive Na+ ion and keep out negatively charged ions such as chloride. The cations flow into a more constricted part of the pore that is 0.3 by 0.5 nm wide, which is just large enough to allow a single Na+ ion with a water molecule associated to pass through. The larger K+ ion cannot fit through this area. Differently sized ions also cannot interact as well with the negatively charged glutamic acid residues that line the pore.

Na+ channels both open and close more quickly than K+ channels, producing an influx of positive charge (Na+) toward the beginning of the action potential and an efflux (K+) toward the end.

Sodium channel blocking agents are any of a class of antiarrhythmic agents that prevent ectopic beats by acting on partially inactivated sodium channels to inhibit abnormal depolarizations.

Sodium channel blockers are used clinically to provide pain relief. Three classes of sodium channel blockers in common clinical use are: local anesthetics such as lidocaine; some anticonvulsants such as phenytoin and carbamazepine, and some antiarrhythmics such as mexiletine. Each of these is known to suppress ectopic peripheral nervous system discharge in experimental preparations and to provide relief in a broad range of clinical neuropathic conditions.

Alkaloid based toxins (e.g. tetrodotoxin (TTX) saxitoxin) block sodium channels by binding to and occluding the extracellular pore opening of the channel. Local anesthetics and Class I antiarrhythmic agents block sodium channels by blocking from the intracellular side of the channel. Akaloid based toxins (e.g. veratridine; batrachotoxin and aconitine); Diterpene based toxins (e.g. grayanotoxin persistently activate (open) sodium channels). Peptide based toxins; (e.g. μ-conotoxin; δ-atracotoxin) modify the gating of sodium channels.

### Chloride Channels

Chloride channels are important for setting cell resting membrane potential and maintaining proper cell volume. These channels conduct Cl− as well as other anions such as HCO3−, I−, SCN−, and NO3−. The structure of these channels is also not other known channels. Chloride channel subunits contain between 1 and 12 transmembrane segments. Some members of this family are activated by voltage, while others are activated by Ca2+, extracellular ligands, and pH among other modulators.

It is now recognized that chloride channels display a variety of important physiological and cellular roles that include regulation of pH, volume homeostasis, organic solute transport, cell migration, cell proliferation and differentiation. A number of different gene products have been shown to function as chloride channels. Based on sequence homology the chloride channels can be subdivided into a number of groups. The importance of one such group, the CLC family of chloride channels, can be seen from the diseases that develop when the channel does not function normally.

Some uses of chloride channel blockers/openers are in the treatment of: Bartter's syndrome, which is associated with renal salt wasting and hypokalemic alkalosis, and is due to the defective transport of chloride ions and associated ions in the thick ascending loop of Henle.

Another inherited disease that affects the kidney organs is Dent's Disease. Thomsen disease is thought to be connected in some way to chloride channel lockers/openers.

### Cholinergic Drugs

Cholinergic drugs produce the same effects as acetylcholine. Acetylcholine is the most common neurohormone of the parasympathetic nervous system, the part of the peripheral nervous system responsible for the every day work of the body. A cholinergic agent, also known as a parasympathomimetic agent, is a chemical which functions to enhance the effects mediated by acetylcholine in the central nervous system, the peripheral nervous system, or both. These include acetylcholine receptor agonists muscarine and nicotine, as well as anticholinesterases.

Suitable cholinergic drugs in accordance with the present invention are selected from a cholinergic agonist of acetylcholine, bethanechol, carbachol, methacholine, and pilocarpine, or an anticholinesterase of ambenonium, neostigmine, physostigmine, pyridostigmine, dyflos, and ecothinopate, and pharmaceutically acceptable salts, isomers, analogs and derivatives thereof.

### Nitric Oxide Donors

Nitric oxide is an inorganic free radical, which has the chemical formula of N═O and abbreviated to NO, and is a remarkably versatile biological messenger. The chemical properties of NO are crucial in defining its biological roles, both as a transcellular signal in the cardiovascular and nervous systems and as a cytotoxic antipathogenic agent released during an inflammatory response. Endogenous NO is synthesized from the amino acid L-arginine by three isoforms of the enzyme NO synthase (NOS). The endothelial (eNOS) and neuronal (nNOS) isoforms that synthesize NO for transcellular signaling are constitutively expressed tightly regulated by a number of cofactors. These NOS isoforms typically synthesize small amounts of NO and require activation by Ca²⁺-calmodulin, making them sensitive to agents and processes that increase intracellular calcium levels. The NO generated diffuses to neighboring target cells where it acts primarily through activation of soluble guanylate cyclase (sGC) to generate cGMP from GTP, and bring about the cellular response through a reduction in intracellular calcium levels.

In an embodiment of the present invention, the nitric oxide donors can be selected from several classes, including, but not limited to inorganic nitrites and nitrates (e.g., sodium nitrite), organic nitrites and nitrates, sodium nitroprusside, molsidomine and its metabolites, diazeniumdiolates, S-nitrosothiols, mesoionic oxatriazole and derivatives thereof, iron-sulphur nitrosyls, Sinitrodil, FK-409 (4-Ethyl-2-[(Z)-hydroxyiminol]-5-nitro-3(E)-hexeneamide) and derivatives thereof and hybrid NO donor drugs.

In an embodiment of the present invention, the organic nitric oxide donor includes at least one organic nitrate, which includes esters of nitric acid and may be an acyclic or cyclic compound. For instance, the organic nitrate may be ethylene glycol dinitrate; isopropyl nitrate; amyl nitrite, amyl nitrate, ethyl nitrite, butyl nitrite, isobutyl nitrite, octyl nitrite, glyceryl-1-mononitrate, glyceryl-1,2-dinitrate, glyceryl-1,3-dinitrate, nitroglycerin, butane-1,2,4-triol-trinitrate; erythrityl tetranitrate; pentaerythrityl tetranitrate; sodium nitroprusside, clonitrate, erythrityl tetranitrate, isosorbide mononitrate, isosorbide dinitrate, mannitol hexanitrate, pentaerythritol tetranitrate, penetrinitol, triethanolamine trinitrate, trolnitrate phosphate (triethanolamine trinitrate diphosphate), propatylnitrate, nitrite esters of sugars, nitrate esters of sugars, nitrite esters of polyols, nitrate esters of polyols, nicorandil, apresoline, diazoxide, hydralazine, hydrochlorothiazide, minoxidil, pentaerythritol, tolazoline, scoparone (6,7-dimethoxycoumarin) and pharmaceutically acceptable salts, isomers, analogs and derivatives thereof.

### Other Related Agents

In one embodiment of the present invention, vasoactive drugs that act via eNOS activity enhancement or are important following nitric oxide release leading to cGMP production and in maintaining the level of cGMP by preventing its degredation by PDE5, such as sildenafil, vardenafil and tadalafil are also regarded “nitric oxide donors.”

In one or more embodiments antibacterial drugs like metronidazole—where its by product inhibits bacterial nuclieic acid synthesis when its nitrate group has been reduced—can also regarded as having an effect via a NO pathway.

The term “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

### Polar Solvents

A “polar solvent” is an organic solvent, typically soluble in both water and oil. Certain polar solvents, for example propylene glycol and glycerin, possess the beneficial property of a heumectant.

In one or more embodiments, the polar solvent is a heumectant.

### Polyol

In one or more embodiments, the polar solvent is a polyol. Polyols are organic substances that contain at least two hydroxy groups in their molecular structure.

In one or more embodiments, the foamable carrier contains at least one diol (a compound that contains two hydroxy groups in its molecular structure). Examples of diols include propylene glycol (e.g., 1,2-propylene glycol and 1,3-propylene glycol), butanediol (e.g., 1,2-butanediol, 1,3-butanediol, 2,3-butanediol and 1,4-butanediol), butynediol, pentanediol (e.g., pentane-1,2-diol, pentane-1,3-diol, pentane-1,4-diol, pentane-1,5-diol, pentane-2,3-diol and pentane-2,4-diol), hexanediol (e.g., hexane-1,6-diol hexane-2,3-diol and hexane-2,56-diol), octanediol (e.g., 1,8-octanediol), neopentyl glycol, 2-methyl-1,3-propanediol, diethylene glycol, triethylene glycol, tetraethylene glycol, dipropylene glycol and dibutylene glycol.

In one or more embodiments, the foamable carrier contains at least one triol (a compound that contains three hydroxy groups in its molecular structure), such as glycerin, butane-1,2,3-triol, butane-1,2,4-triol and hexane-1,2,6-triol.

In one embodiment, the polyol is selected from the group consisting of propylene glycol, hexylene glycol, glycerin, polyethylene glycol and glycofurol. In one or more embodiments, the polyol is a mixture of polyols. In one or more embodiments, the mixture of polyols contains at least one diol and at least one triol. According to certain embodiments the ratio between the diol and triol is between about 9:1 and about 1:1, for example, about 8:1, about 7:1, about 6:1, about 5:1, about 4:1, about 3:1, and about 2:1.

### Polyethylene Glycol

In an embodiment of the present invention, the polar solvent consists of a polymerized ethylene glycol, namely polyethylene glycol, which is also termed “PEG”.

According to still other embodiments, the polar solvent is a polyethylene glycol (PEG) or PEG derivative that is liquid at ambient temperature, including PEG200 (MW (molecular weight) about 190-210 kD), PEG300 (MW about 285-315 kD), PEG400 (MW about 380-420 kD), PEG600 (MW about 570-630 kD) PEG 1000, and higher MW PEGs such as PEG 4000, PEG 6000, PEG 8000 and PEG 10000 and mixtures thereof.

The foamable carrier according to the present invention can contain a single PEG or a mixture of two or more PEGs. PEGs having molecular weight of more that about 1000 possess gelling properties; i.e., they increase the viscosity of a composition. Therefore, by combining PEGs with different molecular weights/melting points, one can attain varying levels of flowability as desirable for the treatment of a given target site. In one embodiment the concentration of the PEG should be in a level that results in viscosity, prior to filling of the composition into aerosol canisters, of less than 12,000 CPs, and more preferably, less than 10,000 CPs. However in another embodiment where the propellant causes a substantial reduction in viscosity the said viscosity may be higher provided the foam with propellant is flowable.

Other non-limiting examples of polar solvents include pyrrolidones, (such as N-methyl-2-pyrrolidone and 1-methyl-2-pyrrolidinone), dimethyl isosorbide, 1,2,6-hexapetriol, dimethyl sulfoxide (DMSO), ethyl proxitol, dimethylacetamide (DMAc) and alpha hydroxy acids, such as lactic acid and glycolic acid.

Polar solvents are known to enhance the penetration of active agent into the skin and through the skin, and therefore, their inclusion in the composition of the present invention can be desirable, despite their undesirable skin drying and irritation potential. There is at one level a commonality between the different polar solvents and their penetration enhancement properties. Lower molecular weight alcohols can sometimes be more potent as a solvent, for example by extracting lipids from the skin layers more effectively, which characteristic can adversely affect the skin structure and cause dryness and irritation. Therefore the selection of lower molecular weight alcohols is ideally avoided.

The identification of a “polar solvent”, as used herein, is not intended to characterize the solubilization capabilities of the solvent for any specific active agent or any other component of the foamable composition. Rather, such information is provided to aid in the identification of materials suitable for use as a part in the foamable compositions described herein.

### Secondary Polar Solvent

Optionally, a secondary polar solvent is added to the foamable composition of the present invention. The secondary polar solvent is selected from a variety of organic solvents that are typically miscible on both water and oil. Examples of polar solvent that can be contained in the foamable carrier of the present invention include dimethyl isosorbide, tetrahydrofurfuryl alcohol polyethyleneglycol ether (glycofurol), DMSO, pyrrolidones, (such as N-Methyl-2-pyrrolidone and 1-Methyl-2-pyrrolidinone), ethyl proxitol, dimethylacetamide (DMAc), PEG-type surfactants, PPG-type surfactants and alpha hydroxy acids, such as lactic acid and glycolic acid and their analogs and derivatives.

### Solubilization and Penetration Enhancement

A “skin penetration enhancer”, also termed herein “penetration enhancer,” is an organic solvent, typically soluble in both water and oil. Examples of penetration enhancer include polyols, such as glycerol (glycerin), propylene glycol, hexylene glycol, diethylene glycol, propylene glycol n-alkanols, terpenes, di-terpenes, tri-terpenes, terpen-ols, limonene, terpene-ol, 1-menthol, dioxolane, ethylene glycol, hexylene glycol, other glycols, sulfoxides, such as dimethylsulfoxide (DMSO), dimethylformanide, methyl dodecyl sulfoxide, dimethylacetamide, dimethylisosorbide, monooleate of ethoxylated glycerides (with 8 to 10 ethylene oxide units), azone (1-dodecylazacycloheptan-2-one), 2-(n-nonyl)-1,3-dioxolane, esters, such as isopropyl myristate/palmitate, ethyl acetate, butyl acetate, methyl proprionate, capric/caprylic triglycerides, octylmyristate, dodecyl-myristate; myristyl alcohol, lauryl alcohol, lauric acid, lauryl lactate ketones; amides, such as acetamide oleates such as triolein; various alkanoic acids such as caprylic acid; lactam compounds, such as azone; alkanols, such as dialkylamino acetates, and admixtures thereof.

According to one or more embodiments, the penetration enhancer is a polyethylene glycol (PEG) or PEG derivative that is liquid at ambient temperature

In many cases, polyols, PEGs and polar solvents possess a high solubilizing power and thus, may enable increased concentrations of a pharmaceutical active agent. Polyols, PEGs and polar solvents are also known for their skin penetration enhancement properties. These properties enable high drug bioavailability in the target area of treatment, resulting in an enhanced therapeutic effect. Occasionally, combinations of a polyol, PEGs and a secondary polar solvent, exhibit an increased permeability across the skin.

Thus, in one or more embodiments, the foamable carrier contains (1) at least one polar solvent, selected from a polyol (selected from a diol and a triol) and PEG; and (2) at least one secondary polar solvent.

In one or more embodiments, the foamable carrier contains (1) a mixture of at least two polyols; and (2) at least one secondary polar solvent. In additional embodiments, the foamable carrier contains a mixture of at least one polyol and at least one PEG. In yet other embodiments, the foamable carrier contains (1) a mixture of at least one polyol and at least one PEG and (2) at least one secondary polar solvent.

According to certain embodiments the ratio between the polyol and/or PEG and the secondary polar solvent is between about 9:1 and about 1:1, for example, about 8:1, about 7:1, about 6:1, about 5:1, about 4:1, about 3:1, and about 2:1.

In certain embodiments, the polyol is selected from the group consisting of propylene glycol, hexylene glycol and glycerin (and mixtures thereof); and the secondary polar solvent is selected from the group consisting of dimethyl isosorbide, diethylene glycol monoethyl ether, a liquid polyethylene glycol and glycofurol.

In certain embodiments, the foamable carrier contains (1) at least one polyol; and (2) dimethyl isosorbide.

Short chain alcohols, such as ethanol and propanol are known as polar solvents. However, according to one or more embodiments, the composition of the present invention is substantially alcohol-free, i.e., free of short chain alcohols. Short chain alcohols, having up to 5 carbon atoms in their carbon chain skeleton and one hydroxyl group, such as ethanol, propanol, isopropanol, butanol, iso-butanol, t-butanol and pentanol, are considered less desirable polar solvents due to their skin-irritating effect.

Thus, in certain embodiments, the composition is substantially alcohol-free and includes less than about 5% final concentration of short chain alcohols, preferably less than about 2%, more preferably less than about 1%. However, in other embodiments, a short chain alcohol can be included in the composition, as long as the ratio between the short chain alcohol and the polyol is less than about 1:4 by weight.

### Potent Solvent

In one or more embodiments of the present invention, the foamable composition includes a potent solvent, in addition to or in place of one of the hydrophobic solvents, polar solvents or emollients of the composition. A potent solvent is a solvent other than mineral oil that solubilizes a specific active agent substantially better than a hydrocarbon solvent such as mineral oil or petrolatum. For example, a potent solvent solubilizes the active agent 5 fold better than a hydrocarbon solvent; or even solubilizes the active agent 10-fold better than a hydrocarbon solvent.

In one or more embodiments of the present invention, the composition includes at least one active agent in a therapeutically effective concentration; and at least one potent solvent in a sufficient amount to substantially solubilize the at least one active agent in the composition. The term “substantially soluble” means that at least 95% of the active agent has been solubilized, i.e., 5% or less of the active agent is present in a solid state. In one or more embodiments, the concentration of the at least one potent solvent is more than about 40% of the at least one solvent of the composition of the present invention; or even more than about 60%.

Non-limiting examples of pairs of active agent and potent solvent include: betamethasone valerate: practically insoluble in mineral oil (<0.01%); soluble more than 1% in glycofurol; Hydrocortisone butyrate: practically insoluble in mineral oil (<0.01%); soluble more than 1% in glycofurol; Metronidazole: practically insoluble in mineral oil (<0.01%); soluble more than 1% in dimethyl isosrbide; Ketoconazole: practically insoluble in mineral oil (<0.01%); soluble more than 1% in glycofurol, propylene glycol and dimethyl isosrbide; Mupirocin: practically insoluble in mineral oil (<0.01%); soluble more than 1% in glycofurol, hexylene glycol, dimethyl isosorbide, propylene glycol and polyethylene glycol 400 (PEG 400); Meloxicam, a nonsteroidal anti-inflammatory agent: practically insoluble in mineral oil (<0.001%); soluble in propylene glycol: 0.3 mg/mL; and in PEG 400: 3.7 mg/mL; and progesterone: practically insoluble in mineral oil (<0.001%); soluble in PEG 400: 15.3 mg/mL.

A non-limiting exemplary list of solvents that can be considered as potent solvents includes polyethylene glycol, propylene glycol, hexylene glycol, butaneediols and isomers thereof, glycerol, benzyl alcohol, DMSO, ethyl oleate, ethyl caprylate, diisopropyl adipate, dimethylacetamide, N-methylpyrrolidone, N-hydroxyethylpyrrolidone, polyvinylpyrrolidone, isosorbide derivatives, such as dimethyl isosorbide, glycofurol and ethoxydiglycol (transcutol) and laurocapram.

The use of a potent solvent in a foam composition provides an improved method of delivering poorly soluble therapeutic agents to a target area. It is known that low drug solubility results in poor bioavailability, leading to decreased effectiveness of treatment. Foam compositions of the present invention, for which the solvent includes a potent solvent, increase the levels of the active agent in solution and thus, provide high delivery and improved therapy.

Potent solvents, as defined herein, are usually liquid. Formulations comprising potent solvents and active agents are generally disadvantageous as therapeutics, since their usage involves unwanted dripping and inconvenient method of application; resulting in inadequate dosing. Surprisingly, the foams of the present invention, which are drip-free, provide a superior vehicle for such active agents, enabling convenient usage and accurate effective dosing.

In one or more embodiments of the present invention the present invention the foamable pharmaceutical composition may additionally include a mixture of two or more of the solvents selected from the group of hydrophobic solvents, silicone oils, emollients, polar solvents and potent solvents in an appropriate proportion as would be appreciated to a person skilled in the art.

In one or more embodiments of the present invention, a PPG alkyl ether may act as a potent solvent.

### Hydrophobic Solvent

Optionally, the foamable carrier further contains at least one hydrophobic solvent. The identification of a “hydrophobic solvent”, as used herein, is not intended to characterize the solubilization capabilities of the solvent for any specific active agent or any other component of the foamable composition. Rather, such information is provided to aid in the identification of materials suitable for use as a part in the foamable compositions described herein.

A “hydrophobic solvent” as used herein refers to a material having solubility in distilled water at ambient temperature of less than about 1 gm per 100 mL, more preferable less than about 0.5 gm per 100 mL, and most preferably less than about 0.1 gm per 100 mL.

In one or more embodiments, the hydrophobic organic carrier is an oil, such as mineral oil, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, unsaturated or polyunsaturated oils, such as olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils; essential oils; and silicone oils, such as dimethicone, cyclomethicone, polyalkyl siloxanes, polyaryl siloxanes, polyalkylaryl siloxanes and polyether siloxane copolymers, polydimethylsiloxanes (dimethicones) and poly(dimethylsiloxane)-(diphenyl-siloxane) copolymers.

### Heumectant

A humectant is a substance that helps retain moisture and also prevents rapid evaporation. Non limiting examples are propylene glycol, propylene glycol derivatives, glycerin, hydrogenated starch hydrosylate, hydrogenated lanolin, lanolin wax, D manitol, sorbitol, sodium 2-pyrrolidone-5-carboxylate, sodium lactate, sodium PCA, soluble collagen, dibutyl phthalate, and gelatin. Other examples may be found in the Handbook of Pharmaceutical Additives published by Gower.

### Moisturizers

A moisturizer, is a substance that helps retain moisture or add back moisture to the skin. Examples are allantoin, petrolatum, urea, lactic acid, sodium PCV, glycerin, shea butter, caprylic/capric/stearic triglyceride, candelilla wax, propylene glycol, lanolin, hydrogenated oils, squalene, sodium hyaluronate and lysine PCA. Other examples may be found in the Handbook of Pharmaceutical Additives published by Gower.

Pharmaceutical compositions of the present invention may in one or more embodiments usefully comprise in addition a humectant or a moisturizer or combinations thereof.

### Polymeric Agent

In one or more embodiments the composition of the present invention contains a polymeric agent. A polymeric agent is assisting the creation of foam having fine bubble structure, which does not readily collapse upon release from the pressurized aerosol can. The polymeric agent serves to stabilize the foam composition and to control drug residence in the target organ. Preferably, the polymeric agent is soluble or readily dispersible in the PEG or polyol; or in the mixture of a PEG or polyol and a secondary polar solvent.

Non-limiting examples of polymeric agents that are soluble or readily dispersible in propylene glycol include hydroxypropylcellulose and carbomer (homopolymer of acrylic acid is crosslinked with an allyl ether pentaerythritol, an allyl ether of sucrose, or an allyl ether of propylene, such as Carbopol® 934, Carbopol® 940, Carbopo® 941, Carbopol® 980 and Carbopol® 981).

Other polymeric agents may also be suitable for use according to the present invention provided that they are soluble or readily dispersible in the polyol, or in the mixture of a polyol and an additional polar solvent.

Exemplary polymeric agents include, in a non-limiting manner, naturally-occurring polymeric materials, such as locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, amine-bearing polymers such as chitosan; acidic polymers obtainable from natural sources, such as alginic acid and hyaluronic acid; chemically modified starches and the like, carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers and the like.

Additional exemplary polymeric agents include semi-synthetic polymeric materials such as cellulose ethers, such as methylcellulose, hydroxypropyl cellulose, hydroxypropyl methylcellulose, hydroxyethyl cellulose, hydroxy propylmethyl cellulose, methylhydroxyethylcellulose, methylhydroxypropylcellulose, hydroxyethylcarboxymethylcellulose, carboxymethyl cellulose, carboxymethylcellulose carboxymethylhydroxyethylcellulose, and cationic celluloses. Polyethylene glycols having molecular weight of 1000 or more (e.g., PEG 1,000, PEG 4,000, PEG 6,000 and PEG 10,000) also possess gelling capacity. While these PEGs are considered as “secondary polar solvents,” as detailed herein, they may also be used as polymeric agents.

Mixtures of the above polymeric agents are also contemplated.

The concentration of the polymeric agent should be selected so that the composition, after filling into aerosol canisters, is flowable, and can be shaken in the canister. In one or more embodiments, the concentration of the polymeric agent is selected such that the viscosity of the composition, prior to filling of the composition into aerosol canisters, is less than 12,000 CPs, and more preferably, less than 10,000 CPs.

### Surface Active Agent

The composition of the present invention further contains a surface-active agent. Surface-active agents (also termed “surfactants”) include any agent linking oil and water in the composition, in the form of emulsion. A surfactant's hydrophilic/lipophilic balance (HLB) describes the emulsifier's affinity toward water or oil. HLB is defined for non-ionic surfactants. The HLB scale ranges from 1 (totally lipophilic) to 20 (totally hydrophilic), with 10 representing an equal balance of both characteristics. Lipophilic emulsifiers form water-in-oil (w/o) emulsions; hydrophilic surfactants form oil-in-water (o/w) emulsions. The HLB of a blend of two emulsifiers equals the weight fraction of emulsifier A times its HLB value plus the weight fraction of emulsifier B times its HLB value (weighted average). In many cases a single surfactant may suffice. In other cases a combination of two or more surfactants is desired. Reference to a surfactant in the specification can also apply to a combination of surfactants or a surfactant system. As will be appreciated by a person skilled in the art which surfactant or surfactant system is more appropriate is related to the vehicle and intended purpose. In general terms a combination of surfactants is usually preferable where the vehicle is an emulsion. In an emulsion environment a combination of surfactants can be significant in producing breakable forms of good quality. It has been further discovered that the generally thought considerations for HLB values for selecting a surfactant or sufactant combination are not always binding for emulsions and that good quality foams can be produced with a surfactant or surfactant combination both where the HLB values are in or towards the lipophilic side of the scale and where the HLB values are in or towards the hydrophilic side of the scale. Surfactants also play a role in foam formation where the foamable formulation is a single phase composition.

According to one or more embodiments the composition contains a single surface active agent having an HLB value between about 2 and 9, or more than one surface active agent and the weighted average of their HLB values is between about 2 and about 9. Lower HLB values may in certain embodiments be more applicable to water in oil emulsions.

According to one or more embodiments the composition contains a single surface active agent having an HLB value between about 7 and 14, or more than one surface active agent and the weighted average of their HLB values is between about 7 and about 14. Mid range HLB values may in certain embodiments be more suitable for oil in water emulsions.

According to one or more other embodiments the composition contains a single surface active agent having an HLB value between about 9 and about 19, or more than one surface active agent and the weighted average of their HLB values is between about 9 and about 19. In a waterless or substantially waterless environment a wide range of HLB values may be suitable.

Preferably, the composition of the present invention contains a non-ionic surfactant. Nonlimiting examples of possible non-ionic surfactants include a polysorbate, polyoxyethylene (20) sorbitan monostearate, polyoxyethylene (20) sorbitan monooleate, a polyoxyethylene fatty acid ester, Myrj 45, Myrj 49, Myrj 52 and Myrj 59; a polyoxyethylene alkyl ether, polyoxyethylene cetyl ether, polyoxyethylene palmityl ether, polyethylene oxide hexadecyl ether, polyethylene glycol cetyl ether, steareths such as steareth 2, brij 21, brij 721, brij 38, brij 52, brij 56 and brij W1, a sucrose ester, a partial ester of sorbitol and its anhydrides, sorbitan monolaurate, sorbitan monolaurate, a monoglyceride, a diglyceride, isoceteth-20 and mono-, di- and tri-esters of sucrose with fatty acids. In certain embodiments, suitable sucrose esters include those having high monoester content, which have higher HLB values.

Non-limiting examples of non-ionic surfactants that have HLB of about 7 to about 12 include steareth 2 (HLB˜4.9); glyceryl monostearate/PEG 100 stearate (Av HLB˜11.2); stearate Laureth 4 (HLB˜9.7) and cetomacrogol ether (e.g., polyethylene glycol 1000 monocetyl ether).

Non-limiting examples of preferred surfactants, which have a HLB of 4-19 are set out in the Table below:

| Surfactant | HLB |
|---|---|
| steareth 2 | ~4.9 |
| glyceryl monostearate/PEG 100 stearate | Av ~11.2 |
| Glyceryl Stearate | ~4 |
| Steareth-21 | ~15.5 |
| peg 40 stearate | ~16.9 |
| polysorbate 80 | ~15 |
| sorbitan stearate | ~4.7 |
| laureth 4 | ~9.7 |
| Sorbitan monooleate (span 80) | ~4.3 |
| ceteareth 20 | ~15.7 |
| steareth 20 | ~15.3 |
| ceteth 20 | ~15.7 |
| Macrogol Cetostearyl Ether | ~15.7 |
| ceteth 2 (Lipocol C-2) | ~5.3 |
| PEG-30 Dipolyhydroxystearate | ~5.5 |
| sucrose distearate (Sisterna SP30) | ~6 |
| polyoxyethylene (100) stearate | ~18.8 |

More exemplary stabilizing surfactants which may be suitable for use in the present invention are found below.

### PEG-Fatty Acid Monoester Surfactants

| Chemical name | Product example name | HLB |
|---|---|---|
| PEG-30 stearate | Myrj 51 | >10 |
| PEG-40 laurate | Crodet L40 (Croda) | 17.9 |
| PEG-40 oleate | Crodet O40 (Croda) | 17.4 |
| PEG-45 stearate | Nikkol MYS-45 (Nikko) | 18 |
| PEG-50 stearate | Myrj 53 | >10 |
| PEG-100 stearate | Myrj 59, Arlacel 165 (ICI) | 19 |

### PEG-Fatty Acid Diester Surfactants:

| Chemical name | Product example name | HLB |
|---|---|---|
| PEG-4 dilaurate | Mapeg .RTM. 200 DL (PPG), | 7 |
| | Kessco .RTM.PEG 200 DL | |
| | (Stepan), LIPOPEG 2-DL (Lipo | |
| | Chem.) | |
| PEG-4 | distearate Kessco .RTM. 200 DS | 5 |
| | (Stepan.sub) | |
| PEG-32 dioleate | Kessco .RTM. PEG 1540 DO | 15 |
| | (Stepan) | |
| PEG-400 dioleate | Cithrol 4DO series (Croda) | >10 |
| PEG-400 disterate | Cithrol 4DS series (Croda) | >10 |
| PEG-20 glyceryl oleate | Tagat .RTM. O (Goldschmidt) | >10 |

### Transesterification Products of Oils and Alcohols

| Chemical name | Product example name | HLB |
|---|---|---|
| PEG-30 castor oil | Emalex C-30 (Nihon Emulsion) | 11 |
| PEG-40 hydrogenated castor oil | Cremophor RH 40 (BASF), | 13 |
| | Croduret (Croda), Emulgin HRE | |
| | 40 (Henkel) | |

### Polyglycerized Fatty Acids, Such as:

| Chemical name | Product example name | LB |
|---|---|---|
| Polyglyceryl-6 dioleate | Caprol .RTM. 6G20 (ABITEC); | 8.5 |
| | PGO-62 (Calgene), PLUROL | |
| | OLEIQUE CC 497 | |
| | (Gattefosse)Hodag | |

### PEG-Sorbitan Fatty Acid Esters

| Chemical name | Product example name | HLB |
|---|---|---|
| PEG-20 sorbitan monolaurate | Tween-20 (Atlas/ICI), Crillet 1 | 17 |
| | (Croda), DACOL MLS 20 | |
| | (Condea) | |
| PEG-20 sorbitan | Tween 40 (Atlas/ICI), Crillet 2 | 16 |
| Monopalmitate | (Croda) | |
| PEG-20 sorbitan | Tween-60 (Atlas/ICI), Crillet 3 | 15 |
| monostearate | (Croda) | |
| PEG-20 sorbitan monooleate | Tween-80 (Atlas/ICI), Crillet 4 | 15 |
| | (Croda) | |

### Polyethylene Glycol Alkyl Ethers

| Chemical name | Product example name | HLB |
|---|---|---|
| PEG-2 oleyl ether | oleth-2 Brij 92/93 (Atlas/ICI) | 4.9 |
| PEG-3 oleyl ether | oleth-3 Volpo 3 (Croda) | <10 |
| PEG-5 oleyl ether | oleth-5 Volpo 5 (Croda) | <10 |
| PEG-10 oleyl ether | oleth-10 Volpo 10 (Croda), Brij | 12 |
| | 96/97 (Atlas/ICI) | |
| PEG-20 oleyl ether | oleth-20 Volpo 20 (Croda), Brij | 15 |
| | 98/99 (Atlas/ICI) | |
| PEG-4 lauryl ether | laureth-4Brij 30 (Atlas/ICI) | 9.7 |
| PEG-23 lauryl ether | laureth-23Brij 35 (Atlas/ICI) | 17 |
| PEG-10 stearyl ether | Brij 76 (ICI) | 12 |
| PEG-2 cetyl ether | Brij 52 (ICI) | 5.3 |

### Sugar Ester Surfactants

| Chemical name | Product example name | HLB |
|---|---|---|
| Sucrose distearate | Sisterna SP50, Surfope 1811 | 11 |

### Sorbitan Fatty Acid Ester Surfactants

| Chemical name | Product example name | HLB |
|---|---|---|
| Sorbitan monolaurate | Span-20 (Atlas/ICI), Crill 1 | 8.6 |
| | (Croda), Arlacel 20 (ICI) | |
| Sorbitan monopalmitate | Span-40 (Atlas/ICI), Crill 2 | 6.7 |
| | (Croda), Nikkol SP-10 (Nikko) | |
| Sorbitan monooleate | Span-80 (Atlas/ICI), Crill 4 | 4.3 |
| | (Croda), Crill 50 (Croda) | |
| Sorbitan monostearate | Span-60 (Atlas/ICI), Crill 3 | 4.7 |
| | (Croda), Nikkol SS-10 (Nikko) | |

In one or more embodiments the surface active agent is a complex emulgator in which the combination of two or more surface active agents can be more effective than a single surfactant and provides a more stable emulsion or improved foam quality than a single surfactant. For example and by way of non-limiting explanation it has been found that by choosing say two surfactants, one hydrophobic and the other hydrophilic the combination can produce a more stable emulsion than a single surfactant. Preferably, the complex emulgator comprises a combination of surfactants wherein there is a difference of about 4 or more units between the HLB values of the two surfactants or there is a significant difference in the chemical nature or structure of the two or more surfactants.

Specific non limiting examples of surfactant systems are, combinations of polyoxyethylene alkyl ethers, such as Brij 59/Brij 100; Brij 52/Brij 10; Steareth 2/Steareth 20; Steareth 2/Steareth 21 (Brij 72/Brij 721); combinations of polyoxyethylene stearates such as Myrj 52/Myrj 59; combinations of sucrose esters, such as Surphope 1816/Surphope 1807; combinations of sorbitan esters, such as Span 20/Span 80; Span 20/Span 60; combinations of sucrose esters and sorbitan esters, such as Surphope 1811 and Span 60; combinations of liquid polysorbate detergents and PEG compounds, such as Tween 80/PEG-40 stearate; methyl glucaso sequistearate; polymeric emulsifiers, such as Permulen (TRI or TR2); liquid crystal systems, such as Arlatone (2121), Stepan (Mild RM1), Nikomulese (41) and Montanov (68) and the like.

In certain embodiments the surfactant is preferably one or more of the following: a combination of steareth-2 and steareth-21 on their own or in combination with GMS; in certain other embodiments the surfactant is a combination of polysorbate 80 and PEG-40 stearate. In certain other embodiments the surfactant is a combination of glyceryl monostearate/PEG 100 stearate. In certain other embodiments the surfactant is a combination of two or more of stearate 21, PEG 40 stearate, and polysorbate 80. In certain other embodiments the surfactant is a combination of two or more of laureth 4, span80, and polysorbate 80. In certain other embodiments the surfactant is a combination of two or more of GMS and ceteareth. In certain other embodiments the surfactant is a combination of two or more of steareth 21, ceteareth 20, ceteth 2 and laureth 4 In certain other embodiments the surfactant is a combination of ceteareth 20 and polysorbate 40 stearate. In certain other embodiments the surfactant is a combination of span 60 and GMS.

In one or more embodiments the stability of the composition can be improved when a combination of at least one non-ionic surfactant having HLB of less than 9 and at least one non-ionic surfactant having HLB of equal or more than 9 is employed. The ratio between the at least one non-ionic surfactant having HLB of less than 9 and the at least one non-ionic surfactant having HLB of equal or more than 9, is between 1:8 and 8:1, or at a ratio of 4:1 to 1:4. The resultant HLB of such a blend of at least two emulsifiers is preferably between about 9 and about 14.

Thus, in an exemplary embodiment, a combination of at least one non-ionic surfactant having HLB of less than 9 and at least one non-ionic surfactant having HLB of equal or more than 9 is employed, at a ratio of between 1:8 and 8:1, or at a ratio of 4:1 to 1:4, wherein the HLB of the combination of emulsifiers is preferably between about 5 and about 18.

In certain cases, the surface active agent is selected from the group of cationic, zwitterionic, amphoteric and ampholytic surfactants, such as sodium methyl cocoyl taurate, sodium methyl oleoyl taurate, sodium lauryl sulfate, triethanolamine lauryl sulfate and betaines.

Many amphiphilic molecules can show lyotropic liquid-crystalline phase sequences depending on the volume balances between the hydrophilic part and hydrophobic part. These structures are formed through the micro-phase segregation of two incompatible components on a nanometer scale. Soap is an everyday example of a lyotropic liquid crystal. Certain types of surfactants tend to form lyotropic liquid crystals in emulsions interface (oil-in-water) and exert a stabilizing effect. Non limiting examples of surfactants with postulated tendency to form interfacial liquid crystals are: phospholipids, alkyl glucosides, sucrose esters, sorbitan esters. In certain embodiments of the present invention surfactants which tend to form liquid crystals may improve the quality of foams produced from compositions of the present invention.

In one or more embodiments the surfactant is a surfactant or surfactant combination is capable of or which tends to form liquid crystals.

In one or more embodiments the at least one surface active agent is liquid.

In one or more embodiments the at least one surface active agent is solid, semi solid or waxy.

It should be noted that HLB values may not be so applicable to non ionic surfactants, for example, with liquid crystals or with silicones. Also HLB values may be of lesser significance in a waterless or substantially non-aqueous environment.

In one or more embodiments the surfactant can be, a surfactant system comprising of a surfactant and a co surfactant, a waxy emulsifier, a liquid crystal emulsifier, an emulsifier which is solid or semi solid at room temperature and pressure, or combinations of two or more agents in an appropriate proportion as will be appreciated a person skilled in the art. Where a solid or semi solid emulsifier combination is used it can also comprise a solid or semi solid emulsifier and a liquid emulsifier.

In one or more embodiments of the present invention, the surface-active agent includes at least one non-ionic surfactant. Ionic surfactants are known to be irritants. Therefore, non-ionic surfactants are preferred in applications including sensitive tissue such as found in most mucosal tissues, especially when they are infected or inflamed. We have surprisingly found that non-ionic surfactants alone can provide formulations and foams of good or excellent quality in the carriers and compositions of the present invention.

Thus, in a preferred embodiment, the surface active agent, the composition contains a non-ionic surfactant. In another preferred embodiment the composition includes a mixture of non-ionic surfactants as the sole surface active agent. Yet, in additional embodiments, the foamable composition includes a mixture of at least one non-ionic surfactant and at least one ionic surfactant in a ratio in the range of about 100:1 to 6:1. In one or more embodiments, the non-ionic to ionic surfactant ratio is greater than about 6:1, or greater than about 8:1; or greater than about 14:1, or greater than about 16:1, or greater than about 20:1. In further embodiments, surface active agent comprises a combination of a non-ionic surfactant and an ionic surfactant, at a ratio of between 1:1 and 20:1.

In one or more embodiments of the present invention, a combination of a non-ionic surfactant and an ionic surfactant (such as sodium lauryl sulphate and cocamidopropylbetaine) is employed, at a ratio of between 1:1 and 20:1, or at a ratio of 4:1 to 10:1; for example, about 1:1, about 4:1, about 8:1, about 12:1, about 16:1 and about 20:1 or at a ratio of 4:1 to 10:1, for example, about 4:1, about 6:1, about 8:1 and about 10:1.

In selecting a suitable surfactant or combination thereof it should be borne in mind that the upper amount of surfactant that may be used may be limited by the shakability of the composition. In general terms, as the amount of non liquid surfactant is increased the shakability of the formulation reduces until a limitation point is reached where the formulation becomes non shakable and unsuitable. Thus in an embodiment of the present invention any effective amount of surfactant may be used provided the formulation remains shakable. In other certain exceptional embodiments the upper limit may be determined by flowability such as in circumstances where the composition is marginally or apparently non shakable. Thus in an embodiment of the present invention any effective amount of surfactant may be used provided the formulation remains flowable.

In certain embodiments of the present invention the amount of surfactant or combination of surfactants is between about 0.05% to about 20%; between about 0.05% to about 15%. or between about 0.05% to about 10%. In a preferred embodiment the concentration of surface active agent is between about 0.2% and about 8%. In a more preferred embodiment the concentration of surface active agent is between about 1% and about 6%.

If the composition as formulated is a substantially non shakable composition it is nevertheless possible as an exception in the scope of the present invention for the formulation to be flowable to a sufficient degree to be able to flow through an actuator valve and be released and still expand to form a good quality foam. This surprising and unusual exception may be due one or more of a number of factors such as the high viscosity, the softness, the lack of crystals, the pseudoplastic or semi pseudo plastic nature of the composition and the dissolution of the propellant into the petrolatum.

In one or more embodiments of the present invention, the surface-active agent includes mono-, di- and tri-esters of sucrose with fatty acids (sucrose esters), prepared from sucrose and esters of fatty acids or by extraction from sucro-glycerides. Suitable sucrose esters include those having high monoester content, which have higher HLB values.

### Foam Adjuvant

Optionally, a foam adjuvant is included in the foamable carriers of the present invention to increase the foaming capacity of surfactants and/or to stabilize the foam. In one or more embodiments of the present invention, the foam adjuvant agent includes fatty alcohols having 15 or more carbons in their carbon chain, such as cetyl alcohol and stearyl alcohol (or mixtures thereof). Other examples of fatty alcohols are arachidyl alcohol (C20), behenyl alcohol (C22), 1-triacontanol (C30), as well as alcohols with longer carbon chains (up to C50). Fatty alcohols, derived from beeswax and including a mixture of alcohols, a majority of which has at least 20 carbon atoms in their carbon chain, are especially well suited as foam adjuvant agents. The amount of the fatty alcohol required to support the foam system is inversely related to the length of its carbon chains. Foam adjuvants, as defined herein are also useful in facilitating improved spreadability and absorption of the composition.

In one or more embodiments of the present invention, the foam adjuvant agent includes fatty acids having 16 or more carbons in their carbon chain, such as hexadecanoic acid (C16) stearic acid (C18), arachidic acid (C20), behenic acid (C22), octacosanoic acid (C28), as well as fatty acids with longer carbon chains (up to C50), or mixtures thereof. As for fatty alcohols, the amount of fatty acids required to support the foam system is inversely related to the length of its carbon chain.

Optionally, the carbon atom chain of the fatty alcohol or the fatty acid may have at least one double bond. A further class of foam adjuvant agent includes a branched fatty alcohol or fatty acid. The carbon chain of the fatty acid or fatty alcohol also can be substituted with a hydroxyl group, such as 12-hydroxy stearic acid.

### Modulating Agent

The term modulating agent is used to describe an agent which can improve the stability of or stabilize a foamable carrier or composition and or an active agent by modulating the effect of a substance or residue present in the carrier or composition.

In one or more embodiments the modulating agent is used in a water in oil or oil in water emulsion. In one or more other embodiments the modulating agent is used in a unique waterless emulsion.

In certain embodiments the substance or residue may for example be acidic or basic and potentially alter pH in an emulsion environment or it may be one or more metal ions which may act as a potential catalyst in an emulsion environment.

In certain other embodiments the substance or residue may for example be acidic or basic and potentially alter an artificial pH in a waterless or substantially non aqueous environment or it may be one or more metal ions which may act as a potential catalyst in a waterless or substantially non aqueous environment.

In one or more embodiments the modulating agent is used to describe an agent which can affect pH in an aqueous solution. The agent can be any of the known buffering systems used in pharmaceutical or cosmetic formulations as would be appreciated by a man of the art. It can also be an organic acid, a carboxylic acid, a fatty acid an amino acid, an aromatic acid, an alpha or beta hydroxyl acid an organic base or a nitrogen containing compound.

For non-aqueous formulations, the acid or base should be effectively soluble in the waterless compositions to a sufficient degree as to have a modulating effect on the non aqueous formulation. For formulations containing some degree of water the modulating agent can be effectively soluble in the aqueous compositions to a sufficient degree as to have a modulating effect on the aqueous formulation.

Non limiting examples of mineral acids are boric acid, hydrochloric acid, phosphoric acid, nitric acid, and sulfuric acid. Non limiting examples of organic acids are acetic acid, ascorbic acid, benzoic acid, lactic acid, succinic acid and tartaric acid

In one or more further embodiments the modulating agent is used to describe an agent, which is a chelating or sequestering or complexing agent that is sufficiently soluble or functional in the solvent to enable it to “mop up” or “lock” metal ions.

In an embodiment modulating agent is used to describe an agent which can effect pH in an aqueous solution the term modulating agent more particularly means an acid or base or buffer system or combinations thereof, which is introduced into or is present in and acts to modulate the ionic or polar characteristics and any acidity or basesity balance of an emulsion carrier, composition, foamable carrier or foamable composition or resultant foam of the present invention.

In other embodiments modulating agent is used to describe an agent which can effect pH in an aqueous solution the term modulating agent more particularly means an acid or base or buffer system or combinations thereof, which is introduced into or is present in and acts to modulate the ionic or polar characteristics and any acidity or basesity balance of a waterless or substantially non aqueous carrier, composition, foamable carrier or foamable composition or resultant foam of the present invention.

The substance or residue can be introduced into the formulation from any one or more of the ingredients, some of which themselves may have acidic or basic properties. For example the polymer or solvent may contain basic residues in which case it may be desirable or beneficial to add an acid. Alternatively the surfactant may contain some acid residues in which case the addition of a base may be desirable and beneficial. In some cases more than one ingredient may contain residues which may ameliorate or compound their significance. For example if one ingredient provided weak acid residues and another stronger acid residues the pH in an emulsion environment (or artificial pH in a waterless environment) should be lower. In contrast if one residue was acid and the other basic the net effect in the formulation maybe significantly reduced. In some circumstances the active ingredient may favor an acidic pH or more significantly may need to be maintained at a certain acidic pH otherwise it may readily isomerize, chemically react or breakdown, in which case introducing acidic components such as an acidic polymer might be of help. In an embodiment of the present invention sufficient modulating agent is added to achieve a pH in which the active agent is preferably stable. In another embodiment of the present invention sufficient modulating agent is added to achieve an artificial pH in which the active agent is preferably stable.

The terms pH, pKa, and pKb, buffers and the like are used in classical measurements of an aqueous solution. Such measurements are artificial in a waterless environment. Nevertheless, reference to and description below of such terms are made for convenience and clarity, since such terms are well defined and understood with reference to aqueous solutions and further due to the lack of an appropriate uniform way of describing and identifying the artificial or virtual pH, pK etc in a waterless environment in relation to the present invention. Although predictions of artificial pH can be made using dilution techniques of measurements of waterless formulations diluted in water they are formulation sensitive and specific and have to be carefully calibrated with complex formulas.

Waterless medium can be polar and protic yet it does not conform to classical ionic behavior.

A buffer, as defined by Van Slyke [Van Slyke, J. Biol. Chem. 52, 525 (1922)], is “a substance which by its presence in solution increases the amount of acid or alkali that must be added to cause unit change in pH.”

A buffer solution is a solution of a definite pH made up in such a way that this pH alters only gradually with the addition of alkali or acid. Such a solution consists of a solution of a salt of the week acid in the presence of the three acid itself. The pH of the solution is determined by the dissociation equilibrium of the free acid.

An acid can be a strong acid or a weak acid. A strong acid is an acid, which is a virtually 100% ionized in solution. In contrast, a week acid is one which does not ionize fully. When it is dissolved in water. The lower the value for pKa, the stronger is the acid and likewise, the higher the value for pKa the weaker is the acid.

A base can be a strong base or a weak base. A strong base is something, which is fully ionic with 100% hydroxide ions. In contrast, a weak base is one which does not convert fully into hydroxide ions in solution. The lower the value for pKb, the stronger is the base and likewise, the higher the value for pKb the weaker is the base.

In one or more embodiments of the present invention the modulating agent comprises an organic compound.

In one or more preferred embodiments of the present invention the chelating agent is selected from the group consisting of ethylenediaminetetraacetic acid (EDTA), diethylenetriaminepentaacetic acid (DTPA), hydroxyethylenediaminetriacetic acid (HEDTA), nitrilotriacetic acid (NTA), O,O′-bis(2-aminoethyl)ethyleneglycol-N, N,N′,N′-tetraacetic acid (EGTA), trans-1,2-diaminocyclohexane-N,N,N′,N′-tetraacetic acid (CyDTA) or a pharmaceutically acceptable salt thereof (normally as a sodium salt), more preferably EDTA, HEDTA and their salts; most preferably EDTA and its salts.

In one or more embodiments of the present invention a preferred non limiting example of the chelating agent is EDTA. Typically, the chelating and sequestering agent is present in the composition at a level of up to about 5.0%, preferably 1.0 percent, by weight, of the composition.

In one or more embodiments of the present invention the modulating agent may also be a preservative or an antioxidant or an ionization agent. Any preservative, antioxidant or ionization agents suitable for pharmaceutical or cosmetic application may be used. Non limiting examples of antioxidants are tocopherol succinate, propyl galate, butylated hydroxy toluene and butyl hydroxy anisol. Ionization agents may be positive or may be negative depending on the environment and the active agent or composition that is to be protected. Ionization agents may for example act to protect or reduce sensitivity of active agents. Non limiting examples of positive ionization agents are benzyl conium chloride, and cetyl pyridium chloride. Non limiting examples of negative ionization agents are sodium lauryl sulphate, sodium lauryl lactylate and phospholipids.

### Microemulsions and Nanoemulsions

Microemulsions and nanoemulsion are monophasic, transparent (or slightly translucent) dispersions of oil and water. Unlike conventional emulsions, microemulsions and nanoemulsion are thermodynamically stable, making them a favorable vehicle for pharmaceutical compositions, which have to maintain stability for long periods of time. They and a method of manufacture are more particularly described in US2006/0233721 which is incorporated herein by way of reference. As will be appreciated by a man of the art the methodology may be adapted according to the type of carrier composition.

### Additional Components

In an embodiment of the present invention, a composition of the present invention includes one or more additional components. Such additional components include but are not limited to anti perspirants, anti-static agents, buffering agents, bulking agents, chelating agents, cleansers, colorants, conditioners, deodorants, diluents, dyes, emollients, fragrances, hair conditioners, humectants, pearlescent aids, perfuming agents, permeation enhancers, pH-adjusting agents, preservatives, protectants, skin penetration enhancers, softeners, solubilizers, sunscreens, sun blocking agents, sunless tanning agents, viscosity modifiers and vitamins. As is known to one skilled in the art, in some instances a specific additional component may have more than one activity, function or effect.

### Kits

In certain embodiments, the foamable composition may be provided in a kit that facilitates its delivery in metered forms or that provides additional flexibility in administering multiple foam compositions. The multiagent kit can include at least a first active agent composition in an aerosol container accommodating a pressurized product and having an outlet capable of releasing the pressurized product as a foam and at least a second active agent composition in an aerosol container accommodating a pressurized product and having an outlet capable of releasing the pressurized product as a foam. The active agent may be selected from the group consisting of a channel agent; a cholinergic agent; a nitric oxide donor, or a related agent. The first composition have a first active agent and may be selected from the group consisting of an oil in water emulsion; a water in oil emulsion; a petrolatum in aqueous emulsion; a petrolatum waterless formulation; a waterless oleaginous formulation; and a waterless polyethylene glycol or a waterless propylene glycol based composition. The second composition may include a second active agent and may be selected from the group consisting of an oil in water emulsion; a water in oil emulsion; a petrolatum in aqueous emulsion; a petrolatum waterless formulation; a waterless oleaginous formulation; and a waterless polyethylene glycol or a waterless propylene glycol based composition, said second composition comprising a second active agent. Typically the two compositions are maintained separately prior to administration, either because the first active agent is not suitable to be stored with the first active agent or the second composition is selected from a platform which is other than the platform selected for the first composition.

The first composition may be an aqueous formulation and the second composition is a non aqueous or substantially non aqueous composition, e.g., the first agent is a cholinergic agent and the second agent is selected from the group consisting of a channel agent; a nitric oxide donor, and a related agent, or the first agent is a nitric oxide doner and the second agent is selected from the group consisting of a channel agent; a cholinergic agent, and a related agent, or the first agent is a channel agent and the second agent is agent is selected from the group consisting of a cholinergic agent, a nitric oxide doner and a related agent, or the first agent is a channel opener and the second agent is a channel blocker or a channel modulator, or the first agent is a channel blocker and the second agent is a channel opener or a channel modulator.

In some embodiments, kit is a container has multiple dispensers. The container may include

(1) at least two aerosol containers,

(2) a dispenser head for use with the multi aerosol dispenser comprising
an actuator, wherein the dispensing head is structured and positioned to be an actuator or comprises an actuator button disposed within the dispensing head to simultaneously actuate the plurality of containers
a flow guide comprising
   a plurality of flow conduits disposed within the flow guide; and
      for each of the plurality of flow conduits, an inlet through a wall of the flow guide connecting with a flow conduit; and an outlet from a flow conduit through a wall of the flow guide;
and for each of the plurality of inlets and containers, a linker, each to link an inlet and a container to allow the contents of the container upon actuation to pass through the inlet and through the flow conduit to reach and pass through the outlet;
   and wherein the flow guide is structured and positioned to allow simultaneous flow communication between each of the plurality of flow conduits and wherein the plurality of outlets are structured and positioned to allow substantially contemporaneously dispensing and/or combining of the content from a plurality of containers external to the dispensing head.

In some embodiments, at least one canister includes a metered dosing means for repeatedly delivering a unified quantified dose of foam, or each canister includes a metered dosing means for repeatedly delivering a unified quantified dose of foam.

In some embodiment, at least one composition further comprises an additional active agent other than a channel agent; a cholinergic agent; a nitric oxide donor, or a related agent;

A subject in need applies a first composition to a target area and then applies a second composition to the target area, and the second composition is applied to the target area after allowing for the first composition to be substantially absorbed. The first or second active agent is a combination of two or more active agents, and the combination is a synergistic combination. The kit may be used to identify which platform compositions provide more effective relief to the subject in need.

The kit may be used to identify which platform compositions provide more effective relief to the subject in need wherein a subject in need applies a first composition to a first target area and then applies a second composition to a second target area. and then observes at which target area the level of relief is more effective, for example, the application regime is daily for a period of up to two weeks, and/or where the first and second target areas are parallel or substantially equivalent.

### Substantially Alcohol-Free

According to one or more embodiments, the foamable composition is substantially alcohol-free, i.e., free of short chain alcohols. This includes less than about 5% final concentration of lower alcohols, preferably less than about 2%, more preferably less than about 1%. Short chain alcohols, having up to 5 carbon atoms in their carbon chain skeleton and one hydroxyl group, such as ethanol, propanol, isopropanol, butaneol, iso-butaneol, t-butaneol and pentanol, are considered less desirable solvents or polar solvents due to their skin-irritating effect.

### Substantially Non Aqueous

In certain cases, the active agent degrades in the presence of water, and therefore, in such cases the present of water in the composition is not desirable. Thus, in certain preferred embodiments, the composition is substantially non-aqueous. The term “substantially non-aqueous” or “substantially waterless” is intended to indicate that the composition has a water content below about 5%, preferably below about 2%, such as below about 1.5%. In certain other preferred embodiments the composition is non aqueous or waterless.

By non aqueous or waterless is meant that the composition contains no or substantially no, free or unassociated or absorbed water. It will be understood by a person of the art that the waterless solvents and substances miscible with them of the present invention can be hydrophilic and can contain water in an associated or unfree or absorbed form and may absorb water from the atmosphere and the ability to do so is its hygroscopic water capacity. It is intended that essentially non-aqueous formulations are included within its scope such that the formulations may have present a small amount of water. In some embodiments the composition ingredients are pretreated to reduce, remove or eliminate any residual or associated or absorbed water.

### Shakability

‘Shakability’ means that the composition contains some or sufficient flow to allow the composition to be mixed or remixed on shaking. That is, it has fluid or semi fluid properties. In some very limited cases possibly aided by the presence of silicone it may exceptionally be possible to have a foamable composition which is flowable but not apparently shakable.

A breakable foam is one that is thermally stable, yet breaks under sheer force.

### Breakability

The breakable foam of the present invention is not “quick breaking”, i.e., it does not readily collapse upon exposure to body temperature environment. Sheer-force breakability of the foam is clearly advantageous over thermally induced breakability, since it allows comfortable application and well directed administration to the target area.

### Propellants

Examples of suitable propellants include volatile hydrocarbons such as butane, propane, isobutane and fluorocarbon gases, or mixtures thereof.

Alcohol and organic solvents render foams inflammable. It has been surprisingly discovered that fluorohydrocarbon propellants, other than chloro-fluoro carbons (CMCs), which are non-ozone-depleting propellants, are particularly useful in the production of a non-flammable foamable composition. A test according to European Standard prEN 14851, titled “Aerosol containers—Aerosol foam flammability test” revealed that compositions containing an organic carrier that contains a hydrophobic organic carrier and/or a polar solvent, which are detected as inflammable when a hydrocarbon propellant is used, become non-flammable, while the propellant is an HFC propellant.

Such propellants include, but are not limited to, hydrofluorocarbon (HFC) propellants, which contain no chlorine atoms, and as such, fall completely outside concerns about stratospheric ozone destruction by chlorofluorocarbons or other chlorinated hydrocarbons. Exemplary non-flammable propellants according to this aspect of the invention include propellants made by DuPont under the registered trademark Dymel, such as 1,1,1,2 tetrafluorethane (Dymel 134), and 1,1,1,2,3,3,3 heptafluoropropane (Dymel 227). HFCs possess Ozone Depletion Potential of 0.00 and thus, they are allowed for use as propellant in aerosol products.

Notably, the stability of foamable emulsions including HFC as the propellant can be improved in comparison with the same composition made with a hydrocarbon propellant.

In one or more embodiments foamable compositions comprise a combination of a HFC and a hydrocarbon propellant such as n-butane or mixtures of hydrocarbon propellants such as propane, isobutane and butane.

Propellant can be added about from 3% to about 25% w/w.

### Composition and Foam Physical Characteristics and Advantages

A pharmaceutical or cosmetic composition manufactured using the foamable carrier of the present invention is very easy to use. When applied onto the afflicted body surface of mammals, i.e., humans or animals, it is in a foam state, allowing free application without spillage. Upon further application of a mechanical force, e.g., by rubbing the composition onto the body surface, it freely spreads on the surface and is rapidly absorbed.

The foamable composition of the present invention is stable, having an acceptable shelf-life of at least one year, or preferably, at least two years at ambient temperature, as revealed in accelerated stability tests. Organic carriers and propellants tend to impair the stability of emulsions and to interfere with the formation of stable foam upon release from a pressurized container. It has been observed, however, that the foamable compositions according to the present invention are surprisingly stable. Following accelerated stability studies, they demonstrate desirable texture; they form fine bubble structures that do not break immediately upon contact with a surface, spread easily on the treated area and absorb quickly.

The composition should also be free flowing, to allow it to flow through the aperture of the container, e.g., and aerosol container, and create an acceptable foam.

Foam quality can be graded as follows:

Grade E (excellent): very rich and creamy in appearance, does not show any bubble structure or shows a very fine (small) bubble structure; does not rapidly become dull; upon spreading on the skin, the foam retains the creaminess property and does not appear watery.

Grade G (good): rich and creamy in appearance, very small bubble size, “dulls” more rapidly than an excellent foam, retains creaminess upon spreading on the skin, and does not become watery.

Grade FG (fairly good): a moderate amount of creaminess noticeable, bubble structure is noticeable; upon spreading on the skin the product dulls rapidly and becomes somewhat lower in apparent viscosity.

Grade F (fair): very little creaminess noticeable, larger bubble structure than a “fairly good” foam, upon spreading on the skin it becomes thin in appearance and watery.

Grade P (poor): no creaminess noticeable, large bubble structure, and when spread on the skin it becomes very thin and watery in appearance.

Grade VP (very poor): dry foam, large very dull bubbles, difficult to spread on the skin.

Topically administrable foams are typically of quality grade E or G, when released from the aerosol container. Smaller bubbles are indicative of more stable foam, which does not collapse spontaneously immediately upon discharge from the container. The finer foam structure looks and feels smoother, thus increasing its usability and appeal.

As further aspect of the foam is breakability. The breakable foam is thermally stable, yet breaks under sheer force. Sheer-force breakability of the foam is clearly advantageous over thermally induced breakability. Thermally sensitive foams immediately collapse upon exposure to skin temperature and, therefore, cannot be applied on the hand and afterwards delivered to the afflicted area.

The foam of the present invention has several advantages, when compared with hydroalcoholic foam compositions, such as described in WO 2004/071479:
(1) Breakability. The foam of the present invention is thermally stable. Unlike hydroalcoholic foam compositions of the prior art, the foam of the present invention is not “quick breaking”, i.e., it does not readily collapse upon exposure to body temperature environment. Sheer-force breakability of the foam is clearly advantageous over thermally induced breakability, since it allows comfortable application and well directed administration to the target area;
(2) Skin drying and skin barrier function. Short chain alcohols are known to dry the skin and impair the integrity of the skin barrier. By contrast, including a film forming agent in the composition of the present invention does not cause unwanted skin barrier damage; and
(3) Irritability. Due to the lack of alcohol and improvement in skin barrier function, skin irritability is eliminated.

Another property of the foam is specific gravity, as measured upon release from the aerosol can. Typically, foams have specific gravity of less than 0.12 g/mL; or less than 0.10 g/mL; or less than 0.08 g/mL, depending on their composition and on the propellant concentration.

### Additional Active Agents

In many cases, the inclusion of an additional therapeutic agents in the foamable pharmaceutical composition of the present invention, contributes to the clinical activity of the calcium channel blocker or a cholinergic agent. Thus, in one or more embodiments, the foamable composition further includes at least one additional therapeutic agent, in a therapeutically effective concentration.

Suitable additional active agents include, but are not limited to, active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, vitamins, vitamin D derivatives, wound healing agents and wart removers. As is known to one skilled in the art, in some instances, a specific active agent may have more than one activity, function or effect.

### Fields of Applications

In an embodiment of the present invention, the foamable composition of the present invention is suitable for treating any inflicted body surface, which responds to the effect of a calcium channel blocker a cholinergic agent or a nitric oxide donor. In one or more embodiments, foamable carrier is suitable for administration to the skin, a body surface, a body cavity or mucosal surface, e.g., the cavity and/or the mucosa of the nose, mouth, eye, ear, respiratory system, vagina or rectum (severally and interchangeably termed herein “target site”). In a preferred embodiment, the composition is suitable for topical administration in and/or around the anal canal.

In specific embodiments, the foamable composition is suitable for the treatment or prophylaxis of a benign anal disorder comprising local application to the anus or the internal anal sphincter.

In an embodiment of the present invention, the foamable composition is suitable for the treatment or prophylaxis of an anal fissure and/or hemorrhoid condition.

In an embodiment of the present invention, the foamable composition is suitable for the treatment or prophylaxis of keloids and hypertrophic scars.

In an embodiment of the present invention, the composition is useful for the treatment of wound, ulcer and burn. This use is particularly important since the composition of the present invention creates a thin, semi-occlusive layer, which coats the damaged tissue, while allowing exudates to be released from the tissue.

In an embodiment of the present invention, the foamable composition is suitable for transdermal or trans-mucosal delivery of a calcium channel blocker a cholinergic agent or a nitric oxide donor.

In an embodiment of the present invention the foamable composition is suitable for transdermal or trans-mucosal delivery of a calcium channel blocker a cholinergic agent or a nitric oxide donor, for the treatment of sexual dysfunction in males (such as erectile dysfunction) and females.

By including an effective amount of an appropriate agent selected from the group consisting of a calcium channel blocker, a cholinergic agent and a nitric oxide donor, and optionally, additional active agents in the composition, the composition of the present invention are useful in treating an animal or a patient having any one of a variety of dermatological disorders (also termed “dermatoses”), such as classified in a non-limiting exemplary manner according to the following groups:
Any one of a variety of dermatological disorders, including dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, ecthyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, grannuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo.

Likewise, the foamable composition of the present invention is suitable for treating a disorder of a body cavity or mucosal surface, e.g., the mucosa of the nose, mouth, eye, ear, respiratory system, vagina or rectum. Non limiting examples of such conditions include chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum.

In light of the expansion and spreading properties of the foamable composition, it is further suitable for the treatment and prevention of post-surgical adhesions. Adhesions are scars that form abnormal connections between tissue surfaces. Post-surgical adhesion formation is a natural consequence of surgery, resulting when tissue repairs itself following incision, cauterization, suturing, or other means of trauma. When comprising appropriate protective agents, the foam is suitable for the treatment or prevention of post surgical adhesions. The use of foam is particularly advantageous because foam can expand in the body cavity and penetrate into hidden areas that cannot be reached by any other alternative means of administration.

Other foamable compositions are described in: U.S. Publication No. 05-0232869, published on Oct. 20, 2005, entitled NONSTEROIDAL IMMUNOMODULATING KIT AND COMPOSITION AND USES THEREOF; U.S. Publication No. 05-0205086, published on Sep. 22, 2005, entitled RETINOID IMMUNOMODULATING KIT AND COMPOSITION AND USES THEREOF; U.S. Publication No. 06-0018937, published on Jan. 26, 2006, entitled STEROID KIT AND FOAMABLE COMPOSITION AND USES THEREOF; U.S. Publication No. 05-0271596, published on Dec. 8, 2005, entitled VASOACTIVE KIT AND COMPOSITION AND USES THEREOF; U.S. Publication No. 06-0269485, published on Nov. 30, 2006, entitled ANTIBIOTIC KIT AND COMPOSITION AND USES THEREOF; U.S. Publication No. 07-0020304, published on Jan. 25, 2007, entitled NON-FLAMMABLE INSECTICIDE COMPOSITION AND USES THEREOF; U.S. Publication No. 06-0193789, published on Aug. 31, 2006, entitled FILM FORMING FOAMABLE COMPOSITION; U.S. patent application Ser. No. 11/732,547, filed on Apr. 4, 2007, entitled ANTI-INFECTION AUGMENTATION OF FOAMABLE COMPOSITIONS AND KIT AND USES THEREOF; U.S. Provisional Patent Application No. 60/789,186, filed on Apr. 4, 2006, KERATOLYTIC ANTIFUNGAL FOAM; U.S. Provisional Patent Application No. 60/815,948, filed on Jun. 23, 2006, entitled FOAMABLE COMPOSITIONS COMPRISING A CALCIUM CHANNEL BLOCKER, A CHOLINERGIC AGENT AND A NITRIC OXIDE DONOR; U.S. Provisional Patent Application No. 60/818,634, filed on Jul. 5, 2006, entitled DICARBOXYLIC ACID FOAMABLE VEHICLE AND PHARMACEUTICAL COMPOSITIONS THEREOF; U.S. Provisional Patent Application No. 60/843,140, filed on Sep. 8, 2006, entitled FOAMABLE VEHICLE AND VITAMIN PHARMACEUTICAL COMPOSITIONS THEREOF, all of which are incorporated herein by reference in their entirety. More particularly any of the active ingredients; the solvents; the surfactants; foam adjuvants; penetration enhancers; humectants; moisturizers; and other excipients as well as the propellants listed therein can be applied herein and are incorporated by reference.

The following examples further exemplify the PPG foamable pharmaceutical carriers, pharmaceutical compositions thereof, methods for preparing the same, and therapeutic uses of the compositions. The examples are for the purposes of illustration only and are not intended to be limiting of the invention. Many variations may be carried out by one of ordinary skill in the art and are contemplated within the full scope of the present invention.

### Methodology

A general procedure for preparing foamable compositions is set out in WO 2004/037225, which is incorporated herein by reference.

### Emulsion Foam

1. Mix oily phase ingredients and heat to 75° C. to melt all ingredients and obtain homogeneous mixture.
2. Mix polymers in water with heating or cooling as appropriate for specific polymer.
3. Add all other water soluble ingredients to water-polymer solution and heat to 75° C.
4. Add slowly internal phase to external phase at 75° C. under vigorous mixing and homogenize to obtain fine emulsion. Alternatively the external phase is added slowly to the internal phase.
5. Cool to below 40° C. and add sensitive ingredients with mild mixing.
6. Cool to room temperature.

### Waterless Foam

1. Dissolve the polymers in the main solvent with heating or cooling as appropriate for specific polymer. Add the all other ingredients and heat to 75° C. to melt and dissolve the various ingredients.
2. Cool to below 40° C. and add sensitive ingredients with mild mixing.
3. Cool to room temperature.

### Oily Waterless Foam

1. Mix all ingredients excluding polymers and heat to 75° C. to melt and dissolve and obtain homogeneous mixture.
2. Mix well and cool to below 40° C. and add the polymers and sensitive ingredients with moderate mixing.
3. Cool to room temperature.
   Oily Foam with Phospholipids and/or Water
1. Swell the phospholipids in the main oily solvent under mixing for at least 20 minutes until uniform suspension is obtained.
2. Add all other ingredients excluding polymers and heat to 75° C. to melt and dissolve and obtain homogeneous mixture.
3. Mix well and cool to below 40° C. and add the polymers and sensitive ingredients with moderate mixing.
4. Cool to room temperature.
5. In case of polymers dissolved in water or organic solvent, dissolve the polymers in the solvent with heating or cooling as appropriate for specific polymer and add to the oily mixture under vigorous mixing at ˜40° C.

Canisters Filling and Crimping
Each aerosol canister is filled with PFF and crimped with valve using vacuum crimping machine.

Pressurizing

Propellant Filling
Pressurizing is carried out using a hydrocarbon gas or gas mixture
Canisters are filled and then warmed for 30 sec in a warm bath at 50° C. and well shaken immediately thereafter.

Closure Integrity Test.
Each pressurized canister is subjected to bubble and crimping integrity testing by immersing the canister in a 60° C. water bath for 2 minutes. Canisters are observed for leakage as determined by the generation of bubbles. Canisters releasing bubbles are rejected.

### Tests

By way of non limiting example the objectives of hardness, collapse time and FTC stability tests are briefly set out below as would be appreciated by a person of the art.

### Hardness

LFRA100 instrument is used to characterize hardness. A probe is inserted into the test material. The resistance of the material to compression is measured by a calibrated load cell and reported in units of grams on the texture analyzer instrument display. Preferably at least three repeat tests are made. The textural characteristics of a dispensed foam can effect the degree of dermal penetration, efficacy, spreadability and acceptability to the user. The results can also be looked at as an indicator of softness. Note: the foam sample is dispensed into an aluminum sample holder and filled to the top of the holder.

### Collapse Time

Collapse time (CT) is examined by dispensing a given quantity of foam and photographing sequentially its appearance with time during incubation at 36° C. It is useful for evaluating foam products, which maintain structural stability at skin temperature for at least 1 min.

### Viscosity

Viscosity is measured with Brookfield LVDV-II+PRO with spindle SC4-25 at ambient temperature and 10, 5 and 1 RPM. Viscosity is usually measured at 10 RPM. However, at about the apparent upper limit for the spindle of ˜>50,000 CP, the viscosity at 1 RPM may be measured, although the figures are of a higher magnitude.

### FTC (Freeze Thaw Cycles)

To check the foam appearance under extreme conditions of repeated cycles of cooling, heating, (first cycle) cooling, heating (second cycle) etc., commencing with −100° C. (24 hours) followed by +400° C. (24 hours) measuring the appearance and again repeating the cycle for up to three times.

### Creaming by Centrifugation:

1. Principle of test
a. The centrifugation used in this procedure serves as a stress condition simulating the aging of the liquid dispersion under investigation. Under these conditions, the centrifugal force applied facilitates the coalescence of dispersed globules or sedimentation of dispersed solids, resulting in loss of the desired properties of the formulated dispersion.

2. Procedure
1.1. Following preparation of the experimental formulation/s, allow to stand at room temperature for ≧24 h.
1.2. Handle pentane in the chemical hood. Add to each experimental formulation in a 20-mL glass vial a quantity of pentane equivalent to the specified quantity of propellant for that formulation, mix and allow formulation to stand for at least 1 h and not more than 24 h.
1.3. Transfer each mixture to 1.5 mL microtubes. Tap each microtube on the table surface to remove entrapped air bubbles.
1.4. Place visually balanced microtubes in the centrifuge rotor and operate the centrifuge at 3,000 rpm for 10 min or at 1,000 rpm for 10 min.

### Penetration—Protocol A

### Objective

The study was designed to evaluate the skin permeability of a non alcoholic liquid foam preparations comprising 5% minoxidil and Regaine Forte Scalp solution, which is a hydro alcoholic formulation also comprising 5% minoxidil. In order to provide maximal effectiveness, it is desirable that the active ingredients reside in the skin, specifically near the root shaft of the hair follicle, below the stratum corneum. Conventional formulations currently available are skin permeable and move across the dermal layer into circulation. The penetration studies demonstrate the superior foam properties of a non-alcoholic liquid foam that concentrates minoxidil at the root shaft and minimizes systemic delivery. See Examples in Section C—Minoxidil Studies.

### Experimental Procedure

### Diffusion Cells

The permeability of porcine skin to calcipotriol was measured in-vitro with a Franz diffusion cell system. The solutions on the receiver side were stirred by externally-driven, Teflon coated magnetic bars.

Three sets of experiments were performed using a diffusion cell system containing 12 cells. In the first set, cells 1-3 and 7-8 were arbitrarily assigned to Regaine Forte and cells 4-6 and 10-12 were assigned to the Test solution 1(Example C11 Formula M 08). In the second set, the cells were switched to be assigned to the other product, i.e., cells 1-3 and 7-8 were arbitrarily assigned to Test solution 1 and cells 4-6 and 10-12 were assigned to Regaine Forte. In the third set, cells 2-3 and 7-8 were arbitrarily assigned to Test solution 1 and cells 5-6 and 10-12 were assigned to Test solution 2 (Example C12 Formula M 09).

### Skin Preparation

Full-thickness porcine skin was excised from fresh ears of approximately seven selected slaughtered white pigs. Skin sections (about 2×2 cm) were cut from the outer side only and subcutaneous fat was removed from the skin sections with a scalpel. Transepidermal water loss measurements (TEWL, Dermalab® Cortex Technology, Hadsund, Denmark) were performed and only those pieces that the TEWL levels were within specification (<15 g/m2h) were mounted in the diffusion cells. About 10-15 pieces could be obtained from each pig's ear (outer side), of which only 4-5 pieces are usually found suitable for testing by the TEWLmeter. The skin was placed with the stratum corneum facing up on the receiver chambers, and then the donor chambers were clamped in place. The receiver chamber, defined as the side facing the dermis, was filled with phosphate buffer (4 mM, pH 7.4).

### Permeation Study

Product specimens (200 mg) were applied on the skin. After 6 hours, samples (1 ml) were taken from the receiver chambers into 2-ml vials, and the exposed skin pieces were extracted with ethanol. The receiver and the skin extract solutions were transferred quantitatively into vials and concentrated by evaporation using DNA mini apparatus. The dry samples were kept at −20° C. until analyzed by HPLC. The analyses were usually performed within two days but never more than 7 days from the sampling time.

### Calculation

The permeating drug quantity per unit of the skin surface area was calculated by using the following formula:

$$Qt / S = \frac{A T \cdot V R E C}{E S T \cdot V E X T \cdot 1.767} μ g \text{/} cm 2$$

where:

AT=Area of peak arising from the sample preparation

EST=Slope of the linear calibration curve ranged between 0.5-10 μg/ml

VREC=Volume of the reconstituted sample after solvent evaporation

VEXT=Volume of the extract solution taken for solvent evaporation

### Penetration—Protocol B

### Objective

The study was designed to evaluate the skin permeability of a non alcoholic Foamix liquid foam preparations comprising 5% minoxidil, Regaine Forte Scalp solution and Rogaine Foam, also comprising 5% minoxidil.

### Experimental Procedure

### Diffusion Cells

The permeability of porcine skin to minoxidil was measured in-vitro with a Franz diffusion cell system. The solutions on the receiver side were stirred by externally-driven, Teflon coated magnetic bars.

Several sets of experiments were performed using a diffusion cell system containing:

1. Four experiments each containing: 3 cells for Foamix formulation (N013) and 3 cells for Regaine. All those experiment results were calculated together as one experiment.

2. One experiment containing: 4 cells for Foamix Foam formulation (6), 3 cells for Rogaine Foam and cells 2 for Regaine solution.

3. One experiment containing: 5 cells for Foamix Foam formulation (4), 4 cells for Rogaine Foam and cells 2 for Regaine solution.

### Skin Preparation

Excised pig ear skin, approximately 3×5 cm, is supplied after dermatome sectioning at a thickness of ˜500 μm and stored at −18° C. until use.

Transepidermal water loss measurements (TEWL, Tewameter TM300) were performed and only those pieces that the TEWL levels were within specification (<15 g/m2h) were mounted in the diffusion cells and then the donor chambers were clamped in place. The receiver chamber, defined as the side facing the dermis, was filled with phosphate buffer (4 mM, pH 7.4).

### Permeation Study

Product specimens (200 mg) were applied on the skin. After 24 hours, samples were taken from the receiver chambers.

Tape-Stripping” Procedure for the Removal of Material Adhered to Skin
Adhere a piece of cello tape on each skin slice within the mold and overlay with 2 Kg weight for about 10 seconds. Remove the weight and transfer the tape into a 50 mL tube containing 3 mL ethanol.
Repeat cello-tape adherence procedure for additional nine times (9 individual cell-tapes) and transfer all nine collected tapes to the same tube.
Repeat cello-tape adherence for additional ten times (10 individual cell-tapes) and transfer all ten tapes to a 50 mL tube containing 3 mL ethanol.

Skin Extraction:
Cut out the skin diffusion area (1.77 cm²) from the skin section, and transfer to a 5-mL tube (tube no. 4) containing 3 mL of the extracting solution indicated in the skin absorption protocol.

The receiver and the skin extract solutions were transferred quantitatively into vials and analyzed by HPLC. The analyses were usually performed within two days from the sampling time.

### Calculation

The permeating drug quantity per unit of the skin surface area was calculated by using the following formula:
According to requirements specified in the percutaneous absorption protocol, study results may presented as shown below:
% API detected: for unabsorbed dose, % penetration (retention) of API and % permeation of API (accumulation in receptor solution)

$$A P I ( \% ) = \frac{A P I detected ( mg ) \times 100}{cell diffusion area ( cm 2 ) \times applied dose ( mg )}$$

Where: cell diffusion area: 1.77 cm²

### Stock Compositions

Non-limiting examples of how stock solutions are made up with and without API. Other stock solutions may be made using the same methodology by simply varying adding or omitting ingredients as would be appreciated by one of the ordinary skills in the art.

The liquefied or gas propellant can be added at a concentration of about 3% to about 25%.

### EXAMPLES

The invention is described with reference to the following examples. This invention is not limited to these examples and experiments. Many variations will suggest themselves and are within the full intended scope of the appended claims.

### Section A

### Example A1

### Foamable Non Aqueous PG Carriers Containing Nifedipine

| | NIF1 | NIF2 | NIF3 |
|---|---|---|---|
| Ingredient | % W/W | % W/W | % W/W |
| Nifedipine | 0.1-3.0% | 0.1-3.0% | 0.1-3.0% |
| Laureth-4 | 2.00 | 2.00 | 2.00 |
| Glyceryl stearate | 4.00 | 4.00 | 3.00 |
| and PEG-100 stearate | | | |
| PEG 4000 | 10.00 | — | — |
| Glycerin anhydrous | — | — | 33.00 |
| Hydroxypropylcellulose (Klucel EF) | 2.00 | 2.00 | 2.00 |
| Propylene glycol (PG) | To 100.00 | To 100.00 | To 100.00 |
| Foam quality | Good | Good | Good |
| Shakability | Shakable | Shakable | Shakable |

Notes:
The compositions are substantially water-free.
Composition NIF2 contains the minimum number of components that constitute a foamable composition, which upon release from an aerosol pressurized container affords foam of Good or Excellent quality. It contains a diol (PG), a polymeric agent (Klucel EF), and a non-ionic surface active agent (PEG-100 stearate and Laureth 4).
Composition NIF1 demonstrates that the addition of 10% PEG (secondary polar solvent) maintains Good foam quality.
Composition NIF3 demonstrates that a mixture of two polyols (PG and glycerin maintains Good foam quality. This composition possesses high hydration and lubrication effect.
The liquefied or gas propellant can be added at a concentration of about 3% to about 25%.

The following procedure was employed when the compositions of Example 1 were produced.

### Step 1: Preparation of Phase A

1. Heat Propylene glycol and stearyl alcohol to 80-85° C.
2. Add Klucel while mixing.
3. Cool to 70-75° C. Add all other ingredients while mixing. Agitation continues until solution uniformity is reached.
4. Add the active agent with moderate mixing.
5. Cool solution to 30° C. with moderate mixing.

### Step 2: Canisters Filling and Crimping

1. Each aerosol canister 35×70 mm is filled with 30±5% g of the composition.
2. Each canister was closed with an aerosol valve, using a vacuum-crimping machine.

### Step 3: Pressurizing

Propellant (a mixture of propane, butane and isobutane) was added to each of the canisters

### Example A2

### Additional Non Aqueous Foamable Compositions Containing Polyols, an Additional Polar Solvent, a Calcium Channel Blocker or a Cholinergic Agent, Having Excellent Foam Structure

| | NIF4 | DIL1 | BET1 | SIL1 | NIFLID1 | NIFMET1 |
|---|---|---|---|---|---|---|
| Ingredient | % w/w | % w/w | % w/w | % w/w | % w/w | % w/w |
| Nlfedipine | 0.2 | | | | 0.3 | 0.3 |
| Dilthiazem | | 2.0 | | | | |
| Bethanechol | | | 1.0 | | | |
| Sildenafil citrate | | | | 1.0 | | |
| Lidocaine | | | | | 2.0 | |
| Metronidazole | | | | | | 0.75 |
| Stearyl Alcohol | 1.0 | 1.0 | 1.0 | 1.0 | 1.0 | 1.0 |
| Klucel EF | 1.5 | 1.5 | 1.5 | 1.5 | 1.5 | 1.5 |
| Laureth-4 | 2.0 | 2.0 | 2.0 | 2.0 | 2.0 | 2.0 |
| Dimethyl Isosorbide | 15.0 | 15.0 | 15.0 | 15.0 | 15.0 | 15.0 |
| Macrogol Cetostearyl Ether | 1.5 | 1.5 | 1.5 | 1.5 | 1.5 | 1.5 |
| Glyceryl Stearate | 1.0 | 1.0 | 1.0 | 1.0 | 1.0 | 1.0 |
| Propylene glycol (PG) | To | To | To | To | To | To |
| | 100.00 | 100.00 | 100.00 | 100.00 | 100.00 | 100.00 |

Notes:
The compositions, as presented in the above table are substantially water-free.
Water can optionally be added, up to 25%.
Composition SIL1 is useful for the treatment of sexual disfunction in males and females. The vehicle is designed for transdermal delivery of the drug.
Composition NIFLID1 is useful for the treatment of anal fissures. It combines a calcium channel blocked and an anesthetic agent in the same formulation.
Composition NIFMET1 is useful for the treatment of rosacea. It combines metronidazole that treats the parasite aspect of rosacea and nifedipine, which treats telangiectasia.
The liquefied or gas propellant can be added at a concentration of about 3% to about 25%.

### Example A3

### Foamable Polyols Compositions Containing Minoxidil

| | MIN1 | MIN2 |
|---|---|---|
| Ingredient | % W/W | % W/W |
| Propylene glycol | 67.00 | 67.00 |
| Polysorbate 60 | 12.00 | 12.00 |
| Klucel GF | 0.50 | 0.50 |
| Purified water | 15.5 | 14.50 |
| Cetearyl alcohol and Cetearyl | | 1.00 |
| glucoside | | |
| Minoxidil | 5.00 | 5.00 |

Notes:
The compositions are useful for the treatment of hair loss.
Composition MIN2 contains a foam adjuvant.
High amount of polisorbate 60 facilitates a more efficaceous treatment.
The liquefied or gas propellant can be added at a concentration of about 3% to about 25%.

### Section B

### Formulations

Group V1 -Non Aqueous Petroleum/Oil

| | 1% w/w | 2% w/w | 3% w/w | 4% w/w | 5% w/w |
|---|---|---|---|---|---|
| Ingredient name | | | | | |
| Petrolatum | 30.00 | — | — | — | — |
| (sofmetic) | | | | | |
| Mineral oil, light | 39.00 | — | — | — | — |
| PPG-15 | 15.00 | — | — | — | — |
| stearyl ether | | | | | |
| Behenyl alcohol | 1.00 | — | — | — | — |
| Cetostearyl | 4.00 | — | — | — | — |
| alcohol | | | | | |
| Ceteth 20 | 4.00 | — | — | — | — |
| Steareth 2 | 3.00 | — | — | — | — |
| Glyceryl stearate | 2.00 | — | — | — | — |
| Aluminum Starch | 2.00 | — | — | — | — |
| Octenylsuccinate | | | | | |
| STOCK PFF | 100.00 | 99.50 | 99.90 | 99.00 | 99.00 |
| TDF005- | | | | | |
| 070612P | | | | | |
| Nifedipine | — | 0.50 | — | — | — |
| Carbamyl-β- | — | — | 0.10 | — | — |
| methylcholine | | | | | |
| chloride | | | | | |
| (bethanechol) | | | | | |
| D-Isosorbide | — | — | — | 1.00 | — |
| dinitrate - | | | | | |
| Lactose mixture | | | | | |
| L-Arginine | — | — | — | — | 1.00 |
| Control: | — | 100.00 | 100.00 | 100.00 | 100.00 |
| Propellant | | 10.00 | 10.00 | 10.00 | 10.00 |
| propane; | | | | | |
| isobutene | | | | | |
| and butane | | | | | |
| mixture | | | | | |
| Result: | | | | | |
| Crystals in PFF | | 1 | 0 | n/a | 1 |
| 1 = Yes; | | | | | |
| 0 = No. | | | | | |
| Foam Quality | | 5 | 5 | 5 | 5 |
| 5 = Good | | | | | |
| Density g/mL | | 0.167 | 0.144 | n/a | 0.159 |
| Shakability | | 1 | 1 | 1 | 1 |
| 1 = Yes | | | | | |
| Collapse time sec | | >300 | >300 | >300 | >300 |

Comment: a petrolatum and mineral oil mixture based foams.

The formulations comprise from about 60% to about 80% petrolatum and mineral oil mixture:

Hydrophobic solvent (PPG15 stearyl ether) from about 1 to about 20%

Stabilizing surfactants about 3 to about 10%

Fatty alcohols and/or acids about 1 to about 10%

Modified starch polymers (ASOS) about 1 to about 10%

In comparative examples, formulations containing 1% L-Arginine did not form a shakeable foam; i.e., it forms a “block” when the oil level was lower with respect to petrolatum. In addition, reducing the PPG 15 stearyl ether to 5% or less reduced formulation liquidity, resulting in a “blocked” foam.

Group VI -Aqueous Petrolatum/oil

| | 1% w/w | 2% w/w | 3% w/w |
|---|---|---|---|
| Ingredient name | | | |
| Petrolatum (sofmetic) | 42.00 | — | — |
| Mineral oil, light | 18.00 | — | — |
| Behenyl alcohol | 1.00 | — | — |
| Cetostearyl alcohol | 2.00 | — | — |
| Ceteth 20 | 2.16 | — | — |
| Sorbitane oleate | 3.84 | — | — |
| Aluminum Starch | 3.00 | — | — |
| Octenylsuccinate | | | |
| Citric acid | 0.18 | — | — |
| Sodium citrate | 0.14 | — | — |
| Water, purified | 27.48 | — | — |
| Sharomix 824 | 0.20 | — | — |
| STOCK VZC010-070612P | 100.00 | 99.00 | 99.90 |
| Diltiazem hydrochloride | — | 1.00 | — |
| Carbamyl-β-methylcholine | — | — | 0.10 |
| chloride (bethanechol) | | | |
| Control: | — | 100.00 | 100.00 |
| Dymel 134a Propellant | — | 10.00 | 10.00 |
| Result: | | | |
| Crystals in PFF | Yes-1/No-0 | n/a | 0 |
| Foam Quality | E-6 . . . VP-1 | Good | Poor |
| Foam Density | g/mL | 0.152 | n/a |
| Foam pH | deluded | 3.9 | 4.19 |
| Shakability | Yes-1/No-0 | 0 | 0 |
| Crystals in Foam | Yes-1/No-0 | 0 | |

Comment: an oil-in-water emulsion base made of petrolatum and mineral oil mixture based foams, surfactants and water less than about 45%.

The formulations comprise from about 40% to about 70% petrolatum and mineral oil mixture:

Stabilizing surfactants about 3% to about 10%

Fatty alcohols and/or acids about 1% to about 10%

ASOS about 1% to about 10%

Add water to 100% (not more then 45%)
May also contain an Hydrophobic solvent (PPG15 stearyl ether, IPM) from about 1% to about 20%

Group IV Oil in Water Emulsion

| Ingredient name | 1 % w/w | 2 % w/w | 3 % w/w | 4 % w/w | 5 % w/w | 6 % w/w | 7 % w/w |
|---|---|---|---|---|---|---|---|
| Mineral oil, light | 6.00 | — | — | — | — | — | — |
| Isopropyl myristate | 6.00 | — | — | — | — | — | — |
| Glyceryl stearate | 0.50 | — | — | — | — | — | — |
| Stearyl alcohol | 1.00 | — | — | — | — | — | — |
| PEG-40 stearate | 3.00 | — | — | — | — | — | — |
| Hypromellose K100M | 0.30 | — | — | — | — | — | — |
| Xanthan gum | 0.30 | — | — | — | — | — | — |
| Polysorbate 80 | 1.00 | — | — | — | — | — | — |
| Water, purified | 81.30 | — | — | — | — | — | — |
| Sharomix 824 | 0.60 | — | — | — | — | — | — |
| STOCK | 100.00 | 99.50 | 99.00 | 99.90 | 99.00 | 99.00 | 97.00 |
| Nifedipine | — | 0.50 | — | — | — | — | — |
| Diltiazem hydrochloride | — | — | 1.00 | — | — | — | — |
| Carbamyl-β-methylcholine | — | — | — | 0.10 | — | — | — |
| chloride (bethanechol) | | | | | | | |
| D-Isosorbide dinitrate - | — | — | — | — | 1.00 | — | — |
| Lactose mixture | | | | | | | |
| L-Arginine | — | — | — | — | — | 1.00 | — |
| NaNO2 | — | — | — | — | — | — | 3.00 |
| Control: | — | 100.00 | 100.00 | 100.00 | 100.00 | 100.00 | 100.00 |
| Propellant--propane, | — | 8.00 | 8.00 | 8.00 | 8.00 | 8.00 | 8.00 |
| isobutane and butane | | | | | | | |
| mixture | | | | | | | |
| Result: | | | | | | | |
| Crystals in PFF | Yes- | Yes | n/a | No | n/a | No | No |
| | 1/No-0 | | | | | | |
| Foam Quality | | 5 | 6 | 5 | 6 | 6 | 6 |
| 6 = Excellent 5 = Good | | | | | | | |
| Foam Density | g/mL | 0.026 | 0.032 | 0.029 | n/a | 0.036 | 0.035 |
| Foam pH | | 5.68 | 5.12 | 5.51 | n/a | 9.58 | 6.98 |
| Shakability | 1 = Yes | 1 | 1 | 1 | 1 | 1 | 1 |
| Collapse time | sec | >300 | n/a | n/a | n/a | >300 | >300 |

Comment: an emollient base which is a typical oil in water emulsion with oils content of about 2 to about 40% stabilized with complex emulgators (surfactants mixture) about 1% to about 10% and stabilizing polymers about 0.2% to about 2%

The emulsion may also comprise fatty acids and/or fatty alcohols about 1% to 5% as foam adjuvant and microbial preservative and buffer adjusting and anti-oxidation agents.

Group III Non Aqueous Petrolatum

| | % w/w | % w/w | |
|---|---|---|---|
| Ingredient name | | | |
| (INCI, USNA) | | | |
| Petrolatum Sofmetic LMF | 50.00 | — | |
| Petrolatum, white | 27.00 | — | |
| Glyceryl stearate | 1.50 | — | |
| Myristyl alcohol | 2.00 | — | |
| Stearyl alcohol | 2.50 | — | |
| Polysorbate 20 | 2.00 | — | |
| Zinc oxide | 15.00 | — | |
| STOCK | 100.00 | 99.00 | |
| Propellant | | | |
| L-Arginine | — | 1.00 | |
| Control: | — | 100.00 | |
| n-Butane | — | 20.00 | |
| Result: | | | |
| Crystals in PFF | | Yes | |
| Foam Quality | | Good | |
| Foam Density | | 0.491 | g/mL |
| Shakability | | Yes | |
| Collapse time | | 240 | sec |

Comment: an anhydrous matrix of petrolatum and mineral oil mixture based foams.

The formulations comprise from about 60% to about 80% petrolatum. Mineral oil can be mixed with the petrolatum:
Zinc oxide about 2% to about 20%;
Hydrophobic solvent (PPG15 stearyl ether, IPM) from about 1% to about 20%; Stabilizing surfactants about 3% to about 10%; Fatty alcohols and/or acids about 1 to about 10%;
Modified starch and lipids mixing polymers such as ASOS about 1% to about 10%

Group II Non Aqueous PEG

| Ingredient name | 1 % w/w | 2 % w/w | 3 % w/w | 4 % w/w | 5 % w/w | 6 % w/w |
|---|---|---|---|---|---|---|
| Polyethylene | 97.50 | — | — | — | — | — |
| glycol 400 | | | | | | |
| Hydroxipropyl | 0.50 | — | — | — | — | — |
| cellulose EF | | | | | | |
| Steareth-2 | 2.00 | — | — | — | — | — |
| STOCK PFF | 100.00 | 99.50 | 99.90 | 99.00 | 99.00 | 97.00 |
| VSC007-070612P | | | | | | |
| Nifedipine | — | 0.50 | — | — | — | — |
| Carbamyl-β- | — | — | 0.10 | — | — | — |
| methylcholine | | | | | | |
| chloride | | | | | | |
| (bethanechol) | | | | | | |
| D-Isosorbide | — | — | — | 1.00 | — | — |
| dinitrate - Lactose | | | | | | |
| mixture | | | | | | |
| L-Arginine | — | — | — | — | 1.00 | — |
| NaNO2 | — | — | — | — | — | 3.00 |
| Control: | — | 100.00 | 100.00 | 100.00 | 100.00 | 100.00 |
| Propellant* | — | 8.00 | 8.00 | 8.00 | 8.00 | 8.00 |
| Result: | | | | | | |
| Crystals in PFF | | n/a | 0 | n/a | 1 | 0 |
| Yes-1/No-0 | | | | | | |
| Foam Quality 5 = | | 6 | 5 | 5 | 5 | 5 |
| Good; 6 = Excellent; | | | | | | |
| Foam Density | | 0.063 | 0.086 | | 0.061 | 0.053 |
| g/mL | | | | | | |
| Shakability 1 = Yes | | 1 | 1 | 1 | 1 | 1 |
| Crystals in Foam | | No | n/a | n/a | n/a | n/a |
| Collapse time sec | | >300 | n/a | n/a | >300 | >300 |
| Hardness g | | 43.39 | n/a | n/a | n/a | n/a |

Comment: liquid and solid Polyethylene glycols mixtures (Polyethylene glycol 400); about 0.2 to about 5%, of a polymer (that is dispersed, swollen or dissolved in the hydrophilic organic solvent); surfactant about 0.2% to about 5%, may also contain anti oxidants and about 2% to about 20% other hydrophilic solvents such as propylene glycol, glycerin, DMI, or water. Propellant is a mixture of propane, isobutene and butane.

| | Group I-PG Non Aqueous with Active Agents | | | | | |
|---|---|---|---|---|---|---|
| Ingredient name | 1 % w/w | 2 % w/w | 3 % w/w | 4 % w/w | 5 % w/w | 6 % w/w |
| Propylene glycol | 46.00 | — | — | — | — | — |
| Hydroxipropyl | 1.50 | — | — | — | — | — |
| cellulose | | | | | | |
| Glycerin anhydrous | 33.00 | — | — | — | — | — |
| Dimethyl isosorbide | 15.00 | — | — | — | — | — |
| Stearyl alcohol | 1.00 | — | — | — | — | — |
| Laureth-4 | 2.00 | — | — | — | — | — |
| Glyceryl | 1.50 | — | — | — | — | — |
| Monostearate & PEG | | | | | | |
| 100 Stearate | | | | | | |
| STOCK PFF | 100.00 | 99.50 | 99.90 | 99.00 | 99.00 | 97.00 |
| Nifedipine | — | 0.50 | — | — | — | — |
| Carbamyl-β- | — | — | 0.10 | — | — | — |
| methylcholine | | | | | | |
| chloride | | | | | | |
| (bethanechol) | | | | | | |
| D-Isosorbide | — | — | — | 1.00 | — | — |
| dinitrate - Lactose | | | | | | |
| mixture | | | | | | |
| L-Arginine | — | — | — | — | 1.00 | — |
| NaNO2 | — | — | — | — | — | 3.00 |
| Control: | — | 100.00 | 100.00 | 100.00 | 100.00 | 100.00 |
| Propellant* | — | 8.00 | 8.00 | 8.00 | 8.00 | 8.00 |
| Result: | | | | | | |
| Crystals in PFF | | n/a | No | n/a | No | No |
| Foam Quality | | Good | Good | Good | Good | Good |
| Foam Density | g/mL | 0.073 | 0.088 | n/a | 0.070 | 0.083 |
| Shakability | | Yes | Yes | Yes | Yes | Yes |
| Crystals in Foam | | Yes | n/a | n/a | Yes | |
| Collapse time | sec | >300 | n/a | n/a | >300 | n/a |
| Hardness | g | 14.78 | n/a | n/a | n/a | n/a |

Comment: A hydrophilic polyol base: propylene glycol, butylene glycol, hexylene glycol, ethylene glycol and mixtures thereof; about 0.2% to about 5% of a polymer (that is dispersed, swollen or dissolved in the hydrophilic organic solvent) about 0.2% to 5%, surfactant may also contain anti oxidants and about 2% to about 20%. other hydrophilic solvents such as glycerin, DMI, polyethylene glycol or water Propellant is a mixture of propane, isobutene and butane.

Group V - Water in Oil Emulsions

| Ingredient name | 1 % w/w | 2 % w/w | 3 % w/w | 4 % w/w | 5 % w/w | 6 % w/w | 7 % w/w |
|---|---|---|---|---|---|---|---|
| PEG-30 | 2.00 | — | — | — | — | — | — |
| Dipolyhydroxystearate | | | | | | | |
| Caprylic/Capric | 9.00 | — | — | — | — | — | — |
| triglyceride | | | | | | | |
| Stearic acid | 1.00 | — | — | — | — | — | — |
| Ceteth-2 | 3.00 | — | — | — | — | — | — |
| Sorbitane oleate | 2.00 | — | — | — | — | — | — |
| PPG-15 Stearyl Ether | 1.00 | — | — | — | — | — | — |
| Isopropyl Isostearate | 9.00 | — | — | — | — | — | — |
| Propylene glycol | 4.00 | — | — | — | — | — | — |
| Magnesium Sulfate | 0.70 | — | — | — | — | — | — |
| Water, purified | 68.00 | — | — | — | — | — | — |
| Sharomix 824 | 0.30 | | | | | | |
| STOCK PFF VZC005- | 100.00 | 99.50 | 99.00 | 99.90 | 99.00 | 99.00 | 97.00 |
| 070614P | | | | | | | |
| Nifedipine | — | 0.50 | — | — | — | — | — |
| Diltiazem | — | — | 1.00 | — | — | — | — |
| hydrochloride | | | | | | | |
| Carbamyl-β- | — | — | — | 0.10 | — | — | — |
| methylcholine | | | | | | | |
| chloride (bethanechol) | | | | | | | |
| D-Isosorbide dinitrate - | — | — | — | — | 1.00 | — | — |
| Lactose mixture | | | | | | | |
| L-Arginine | — | — | — | — | — | 1.00 | — |
| NaNO2 | — | — | — | — | — | — | 3.00 |
| Control: | — | 100.00 | 100.00 | 100.00 | 100.00 | 100.00 | 100.00 |
| Propellant* | — | 8.00 | 8.00 | 8.00 | 8.00 | 8.00 | 8.00 |
| Result: | | | | | | | |
| Crystals in PFF | | 1 | n/a | 0 | n/a | 0 | 1 |
| 1 = Yes; 0 = No. | | | | | | | |
| Foam Quality | — | 5 | 5 | 5 | 5 | 5 | 5 |
| 5 = Good | | | | | | | |
| Foam Density g/mL | | 0.097 | 0.132 | 0.125 | n/a | 0.198 | 0.113 |
| Shakability | | 1 | 1 | 1 | 1 | 1 | 1 |
| 1 = Yes | | | | | | | |
| Crystals | | n/a | No | n/a | n/a | n/a | n/a |
| Collapse time sec | | >300 | n/a | n/a | n/a | >300 | >300 |

Comment: a water-in-oil emulsion that comprises a major stabilizer: PEG-30 Dipolyhydroxystearate about 0.5% to about 10%. The oils content is about 20% to about 80% stabilized with complex emulgators (surfactants mixture) about 1% to about 10%. The emulsion may also comprise fatty acids and/or fatty alcohols about 1% to about 5% as foam adjuvant and microbial preservative and buffer adjusting and anti-oxidation agents. Propellant is a mixture of propane, isobutene and butane.

Group VII Non Aqueous Oil Mixtures

| Ingredient name | 1 % w/w | 2 % w/w | 3 % w/w | 4 % w/w | 5 % w/w | 6 % w/w |
|---|---|---|---|---|---|---|
| Light mineral oil | 22.00 | — | — | — | — | — |
| PPG-15 Stearyl ether | 15.00 | — | — | — | — | — |
| Octyldodecanol | 12.00 | — | — | — | — | — |
| Diisopropyl adipate | 8.00 | — | — | — | — | — |
| Cyclomethicone | 3.00 | — | — | — | — | — |
| Glyceryl stearate | 4.00 | — | — | — | — | — |
| Hydrogenated | 1.50 | — | — | — | — | — |
| Castor Oil | | | | | | |
| Myristyl alcohol | 1.00 | — | — | — | — | — |
| Stearyl alcohol | 3.00 | — | — | — | — | — |
| Oleyl alcohol | 10.00 | — | — | — | — | — |
| Methyl Glucose | 3.00 | — | — | — | — | — |
| Sesquistearate | | | | | | |
| Steareth-2 | 5.00 | — | — | — | — | — |
| Steareth-20 | 2.50 | — | — | — | — | — |
| Aluminum Starch | 10.00 | — | — | — | — | — |
| Octenylsuccinate | | | | | | |
| STOCK PFF | 100.00 | 99.50 | 99.90 | 99.00 | 99.00 | 97.00 |
| Nifedipine | — | 0.50 | — | — | — | — |
| Carbamyl-β- | — | — | 0.10 | — | — | — |
| methylcholine | | | | | | |
| chloride | | | | | | |
| (bethanechol) | | | | | | |
| D-Isosorbide | — | — | — | 1.00 | — | — |
| dinitrate—Lactose | | | | | | |
| mixture | | | | | | |
| L-Arginine | — | — | — | — | 1.00 | — |
| NaNO2 | — | — | — | — | — | 3.00 |
| Control: | — | 100.00 | 100.00 | 100.00 | 100.00 | 100.00 |
| Propellant* | — | 12.00 | 12.00 | 12.00 | 12.00 | 12.00 |
| Result: | | | | | | |
| Crystals in PFF | | 0 | 1 | n/a | 1 | 0 |
| Yes = 1/No = 0 | | | | | | |
| Foam Quality | | 5 | 5- | 5 | 5 | 5 |
| 5 = Good | | | | | | |
| Foam Density g/mL | | 0.210 | 0.195 | n/a | 0.159 | 0.175 |
| Shakability 1 = Yes | | 1 | 1 | 1 | 1 | 1 |
| Collapse time sec | | 170 | n/a | n/a | >300 | 160 |

Comment: an anhydrous matrix of hydrophobic solvents, oils esters and/or ethers (Octyl dodecanol, PPG-15 stearyl ether, IPM, DISPA), and mineral oil from about 60% to about 90%
Stabilizing surfactants about 3% to about 10%
Fatty alcohols and/or acids about 1% to about 10%
Modified starch polymers (ASOS) about 1% to about 10%. Propellant is a mixture of propane, isobutene and butane.

### Section C

### Minoxidil Formulations

### Example C1

### 20% PG; 15% Polysorbate 60; Aqueous Foam Compositions

### Part A—Formulations

| | MFP-001 | MFP-002 | MFP-009 | MFP-010 | MFP-003 |
|---|---|---|---|---|---|
| Ingredients | | | | | |
| Propylene | 20.00 | 20.00 | 20.00 | 20.00 | 20.00 |
| glycol | | | | | |
| Polysorbate | 15.00 | 15.00 | 15.00 | 15.00 | 15.00 |
| 60 | | | | | |
| Lactic acid | 18.00 | 10.00 | 10.00 | 10.00 | 13.00 |
| Purified | 36.40 | 42.40 | 37.40 | 34.40 | 26.40 |
| water | | | | | |
| Xanthan | 0.30 | 0.30 | 0.30 | 0.30 | 0.30 |
| gum | | | | | |
| Methocel | 0.30 | 0.30 | 0.30 | 0.30 | 0.30 |
| A4M | | | | | |
| Minoxidil | 10.00 | 12.00 | 17.00 | 20.00 | 25.00 |
| Total | 100.00 | 100.00 | 100.00 | 100.00 | 100.00 |
| Sodium | To pH | To pH | To pH | To pH | To pH |
| hydroxide | 4.5-5.0 | 4.5-5.0 | 4.5-5.0 | 4.5-5.0 | 4.5-5.0 |
| Propellant: | 8% | 8% | 8% | 8% | 8% |
| [propane: | | | | | |
| isobutane: | | | | | |
| n-butane] | | | | | |
| mixture | | | | | |
| Results | | | | | |
| Appearance | | | | | |
| Quality | G-E | G-E | G-E | G-E | G-E |
| Color | White | White | White | White | White |
| Odor | No odor | No odor | No odor | No odor | No odor |
| Shakability | Good | Good | Good | Good | Good |
| Ph | 4.60 | 4.54 | 4.54 | 4.53 | 4.58 |
| Microscope | no | no | no | no | crystals |
| | crystals | crystals | crystals | crystals | |

Comment:

Solubility of minoxidil in 20% PG formulations was investigated. Using acid to form minoxidil salts it was possible to increase the level of minoxidil to about 20% of the formulation without any crystallization. Once the concentration was increased further to 25% the minoxidil crystallized out. Thus, the maximum level depending on the formulation used is somewhere about 20% to about 25% minoxidil. The lactic acid is relatively strong and so base is added to raise the pH to about 4.5. The formulation may be prepared with lower levels of acid. All the formulations achieved good to excellent quality foam and had a pleasant skin feeling.

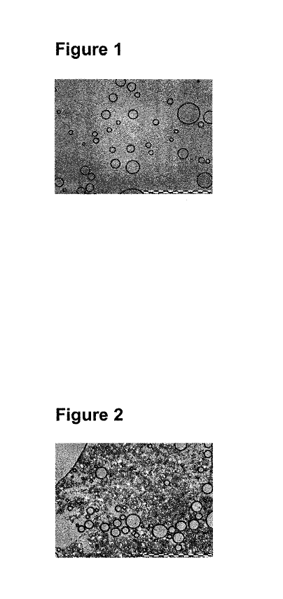

FIG. 1 shows a photo micrograph of foam formulation 10 after shear forces disclosing no crystals.

FIG. 2 is a photomicrograph of a foam prepared from Formulation MFP-003 after shear forces. Note that minosidil crystals are clearly seen.

### Example C2

### Part A

### Formulations

40% and 50% PG; 15% and 10% Polysorbate 60; Aqueous Foam Compositions.

| | 4 | 22 |
|---|---|---|
| Ingredients | | |
| Propylene glycol | 40.00 | 50.00 |
| Polysorbate 60 | 15.00 | 10.00 |
| Lactic acid | 10.00 | |
| Citric acid | | 5.00 |
| Purified water | 29.40 | 29.40 |
| Xanthan gum | 0.30 | 0.30 |
| Methocel A4M | 0.30 | 0.30 |
| Minoxidil | 5.00 | 5.00 |
| Total | 100.00 | 100.00 |
| Sodium hydroxide | To pH | To pH |
| | 4.5-5.0 | 4.5-5.0 |
| Propellant (propane; | 8% | 8% 1681 |
| isobutene and butane | | |
| mixture) | | |
| Results | | |
| Appearance | | |
| Quality | G-E | G-E |
| Color | White | White |
| Odor | No odor | No odor |
| Shakability | Good | Good |
| Ph | 4.63 | 4.51 |
| Microscope | no crystals | no crystals |

Comment:

The concentration of PG was doubled to 40%. The formulation produced a good to excellent quality foam with a pleasant skin feeling. By way of general comment and observation as the level of PG is further increased (with an equivalent decrease in water) it becomes more difficult to achieve a pleasant skin feeling. Thus, as can be seen with the 50% PG formulation the level of water can be maintained by reducing the levels of Polysorbate 60 and acid (although this is not essential). Note lactic acid was replaced by citric acid.

### Part B

### Penetration Study

| | Average in % of applied dose | | |
|---|---|---|---|
| | 4 | Rogaine Foam | Regaine Forte |
| | (n = 5) | (n = 4) | (n = 2) |
| Tape 1 | 85.3 | 69.1 | 87.8 |
| Tape 2 | 2.20 | 3.40 | 3.99 |
| Tape 3 | 0.50 | 1.00 | 0.58 |
| SE* | 0.60 | 0.60 | 0.69 |
| RC (as is) | 2.20 | 8.20 | 0.16 |

*Tape 1 = Surface; Tape 2 = Upper Stratum Corneum; Tape 3 = Lower Stratum Corneum; SE = is the extract of the skin remaining after removal of Tapes1, 2, and 3, and includes the hair follicle root.

Comment:

The penetration study results (presented as a mean of “n” repeat experiments) disclose that the penetration in the remaining skin including hair root and follicle after removal of the surface and stratum corneum with only 40% PG is not dissimilar to that of the commercial hair preparations, However, in one of the commercial preparations much more of the active drug penetrated through the skin. See Methodology—Protocol B

### Example C3

### 15% Polysorbate 60; with and without 10% PG; Aqueous Foam Compositions

| | 5 | 21 |
|---|---|---|
| Ingredients | | |
| Propylene glycol | 10.00 | |
| Polysorbate 60 | 15.00 | 15.00 |
| Lactic acid | 10.00 | |
| Citric acid | | 7.00 |
| Purified water | 59.40 | 72.40 |
| Xanthan gum | 0.30 | 0.30 |
| Methocel A4M | 0.30 | 0.30 |
| Minoxidil | 5.00 | 5.00 |
| Total | 100.00 | 100.00 |
| Sodium hydroxide | To pH | To pH |
| | 4.5-5.0 | 4.5-5.0 |
| Propellant (propane; | 8% | 8% 1681 |
| isobutene and butane | | |
| mixture) | | |
| Results | | |
| Appearance | | |
| Quality | G-E | G-E |
| Color | White | White |
| Odor | No odor | No odor |
| Shakability | Good | Good |
| Ph | 4.51 | 4.52 |
| Microscope | no crystals | no crystals |

Comment:

The concentration of PG was reduced to 10%. The formulation produced a good to excellent quality foam with a pleasant skin feeling. Satisfactory foam may be made using lower concentrations of PG. It is possible to achieve a foam formulation of good to excellent quality without PG.

### Example C4

### 20% PG; 15% Polysorbate 60; Aqueous Foam Composition that Underwent FTC Cycles

### Part-A

| | 6 |
|---|---|
| Ingredients | |
| Propylene glycol | 20.00 |
| Polysorbate 60 | 15.00 |
| Lactic acid | 10.00 |
| Purified water | 49.40 |
| Xanthan gum | 0.30 |
| Methocel A4M | 0.30 |
| Minoxidil | 5.00 |
| Total | 100.00 |
| Sodium hydroxide | To pH |
| | 4.5-5.0 |
| Propellant (propane; | 8% |
| isobutene and butane | |
| mixture) | |
| Results | |
| Appearance | |
| Quality | G-E |
| Color | White |
| Odor | Very faint odor |
| Shakability | Good |
| Ph | 4.55 |
| Microscope | no crystals |
| Density (g/ml) | 0.077 |
| Collapse time (sec.) | >300 |
| Expansion time (sec.) | 25 |
| Hardness (g) | 15.00 |
| Viscosity (Cp) | 1972 |
| FTC (2 cycles) | G-E |
| | White |
| | Very faint odor |
| | Good |

Comment:

The formulation achieved a good to excellent quality foam and had a pleasant skin feeling. No crystals were observed. The formulation withstood two FTC cycles indicating that the foam is physically resistant to aging.

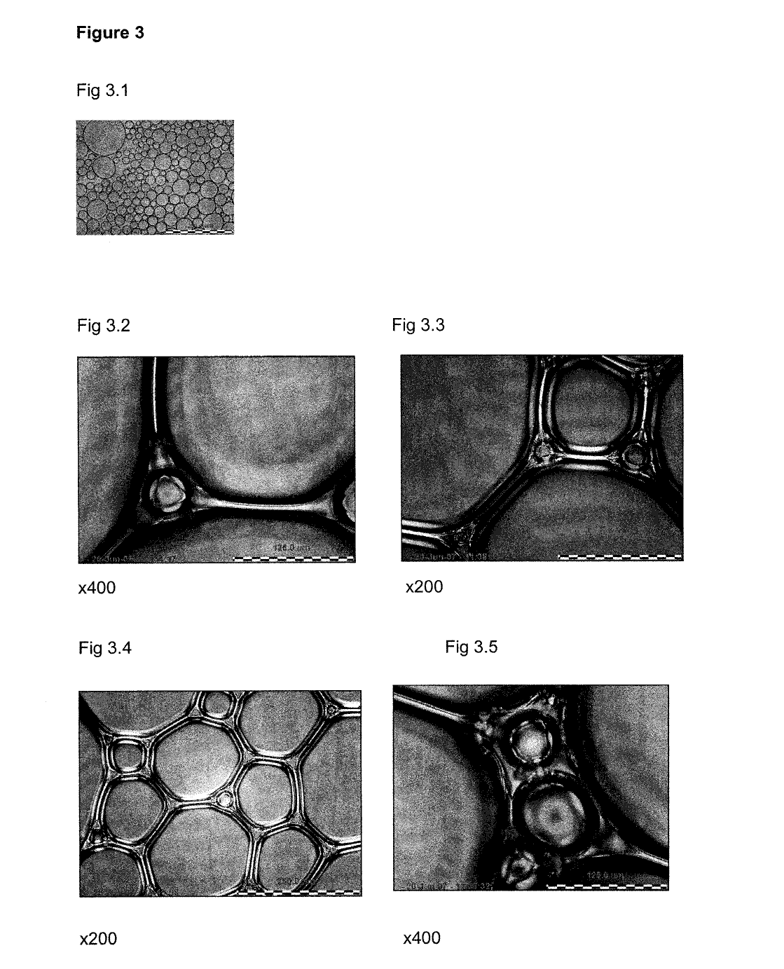

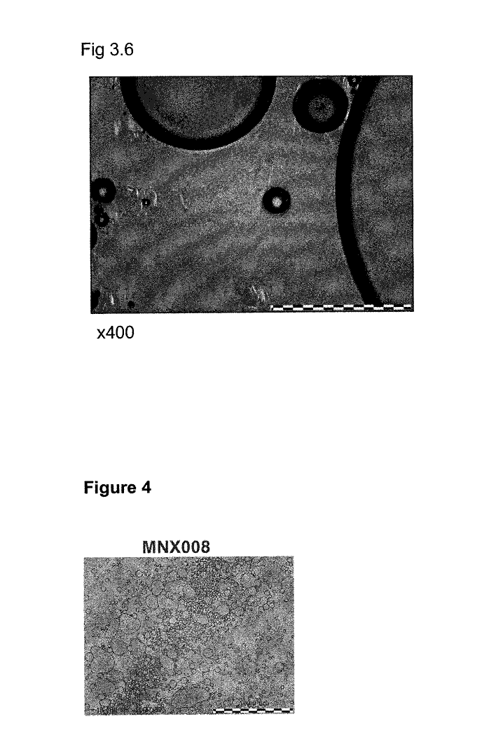

In FIGS. 3.1-3.6, photo micrograph images of Formulation MFP-006 comprising 5% Minoxidil were taken at different magnifications and different locations in the foam disclosing that the foam comprises liquid crystals which disappear after being subject to shear force. No regular minoxidil crystals were observed. Magnification in each figure is 200× or 400× as noted; an optical filter was used and all pictures were taken under polarized light.

The pictures above are light microscope observations of freshly actuated foam formulation MFP-006 and the foam formulation is arranged around large air bubbles. The large shapes are air bubbles and the space between the air bubbles is the formulation, which is arranged in an ordered manner and show birefringence which indicates ordered structures of apparently lamellar types of molecular aggregation resulting in liquid crystalline structures. The minoxidil is believed dissolved in the liquid crystals.

The picture in FIG. 3.6 is de-aerated or mechanically collapsed foam examined under polarized light.

The tremendous interfacial area of formulation and air is collapsed and large ordered birefrigerence aggregates of about 25 microns are detected under the polarized light. Two types of aggregates are identified, the orange and the blue color. These are distinct from Minoxidil crystals by shape, color and appearance.

### Part-D—Penetration

Average in % of applied dose

| | 6 | Rogaine Foam | Regaine Forte |
|---|---|---|---|
| | (4 replicates) | (3 replicates) | (2 replicates) |
| Tape 1 | 65.62 | 55.00 | 45.39 |
| Tape 2 | 2.10 | 2.15 | 1.65 |
| Tape 3 | 0.47 | 0.42 | 0.44 |
| SE | 0.31(±0.093 | 0.57(±0.341 | 0.54 |
| | Standard | Standard | |
| | Error) | Error) | |
| RC | 1.14 | 8.58 | 0.95 |
| (as is) | | | |
| Total | 4.02 | 11.72 | 3.57 |
| without | | | |
| tape 1 | | | |
| (Tape | | | |
| 2, 3, SE | | | |
| and RC) | | | |
| Total skin | 2.88 | 3.14 | 2.62 |
| (Tape 2, 3 | | | |
| and SE) | | | |
| Total | 69.64 | 66.72 | 48.96 |
| Mass | | | |
| balance | | | |

* Tape 1 = Surface; Tape 2 = Upper Stratum Corneum; Tape 3 = Lower Stratum Corneum; SE = is the extract of the skin remaining after removal of Tapes1, 2, and 3, and includes the hair follicle root.
*

Comment:

The penetration study results (presented as a mean of three repeat experiments) disclose that the penetration in the remaining skin including hair root and follicle after removal of the surface and stratum corneum with only 20% PG is not dissimilar to that of the commercial hair preparations taking into account the standard error and sample size, although the overall skin penetration was of a similar amount with all three preparations. However, in one of the commercial preparations much more of the active drug penetrated through the skin. Rogaine foam is a quick break foam and quickly collapses upon application to the body in contrast to the current 20% PG formulation which has a collapse time of >300 seconds. See Methodology—Protocol B

### Example C5

### Use of Acids in 20% PG; 15% Polysorbate 60; Aqueous Foam Composition

| | 7 | 6 | 8 |
|---|---|---|---|
| Ingredients | | | |
| Propylene glycol | 20.00 | 20.00 | 20.00 |
| Polysorbate 60 | 15.00 | 15.00 | 15.00 |
| Lactic acid | | 10.00 | |
| Citric acid | 10.00 | | |
| Stearic acid | | | 10.00 |
| Purified water | 49.40 | 49.40 | 49.40 |
| Xanthan gum | 0.30 | 0.30 | 0.30 |
| Methocel A4M | 0.30 | 0.30 | 0.30 |
| Minoxidil | 5.00 | 5.00 | 5.00 |
| Total | 100.00 | 100.00 | 100.00 |
| Sodium hydroxide | To pH | To pH | To pH |
| | 4.5-5.0 | 4.5-5.0 | 4.5-5.0 |
| Propellant (propane; | 8% | 8% | 8% |
| isobutene and butane | | | |
| mixture) | | | |
| Results | | | |
| Appearance | | | |
| Quality | G-E | G-E | BLOCK |
| Color | White | White | |
| Odor | No odor | Very faint | |
| | | odor | |
| Shakability | Good | Good | |
| Ph | 4.59 | 4.55 | 5.71 |
| Microscope | no crystals | no crystals | crystals |

Comment:

Short chain organic acids that are soluble in water are suitable to increase the solubility of minoxidil in the formulation, such as citric and lactic acid. However organic acids, such as tartaric acid (with a second pKa), crystallizes out of solution when the pH is raised above the first pKa value of 3.02. Boric acid also crystallizes out of solution. Longer chain acids such as stearic acid are relatively weak and as such are needed in higher concentrations that may produce foamable formulations that are non shakable. Also non organic acids which are water soluble may also be suitable, for example hydrochloric acid, phosphoric acid, sulfuric acid.

### Example C6

### 20% PG; 15% Polysorbate 60; Aqueous Foam Composition with 1% Urea

| | 11 |
|---|---|
| Ingredients | |
| Propylene glycol | 20.00 |
| Polysorbate 60 | 15.00 |
| Lactic acid | 10.00 |
| Urea | 1.00 |
| Purified water | 48.40 |
| Xanthan gum | 0.30 |
| Methocel A4M | 0.30 |
| Minoxidil | 5.00 |
| Total | 100.00 |
| Sodium hydroxide | To pH |
| | 4.5-5.0 |
| Propellant (propane; | 8% |
| isobutene and butane | |
| mixture) | |
| Results | |
| Appearance | |
| Quality | G-E |
| Color | White |
| Odor | No odor |
| Shakability | Good |
| Ph | 4.56 |
| Microscope | no crystals |

Comment:

Addition of 1% urea, which possesses both keratolytic and skin-hydration properties that are beneficial to damaged tissue of the skin, did not cause the minoxidil to crystallize out of the formulation.

### Example C7

### 20% PG; 15% Polysorbate 60; Aqueous Foam Composition with and without Polymer (Microcrystalline Cellulose)

| | 12 | 13 |
|---|---|---|
| Ingredients | | |
| Propylene glycol | 20.00 | 20.00 |
| Polysorbate 60 | 15.00 | 15.00 |
| Lactic acid | 10.00 | 8.00 |
| Purified water | 50.00 | 50.00 |
| Avicel 581 | | 2.00 |
| Minoxidil | 5.00 | 5.00 |
| Total | 100.00 | 100.00 |
| Sodium hydroxide | To pH | To pH |
| | 4.5-5.0 | 4.5-5.0 |
| Propellant (propane; | 8% | 8% |
| isobutene and butane | | |
| mixture) | | |
| Results | | |
| Appearance | | |
| Quality | G-E | G-E |
| Color | White | White |
| Odor | No odor | No odor |
| Shakability | Good | Good |
| Ph | 4.54 | |
| Microscope | no crystals | no crystals |

Comment:

Polymer is not essential

### Example C8

### 20% PG; Aqueous Foam Composition with and without Polysorbate 60

| | 14 | 16 | 17 | 6 | 18 |
|---|---|---|---|---|---|
| Ingredients | | | | | |
| Propylene glycol | 28.00 | 27.00 | 25.00 | 20.00 | 15.00 |
| Polysorbate 60 | | | 5.00 | 15.00 | 25.00 |
| Lactic acid | 10.00 | 10.00 | 10.00 | 10.00 | 10.00 |
| Steareth 21 | | 2.00 | | | |
| Purified water | 56.40 | 55.40 | 54.40 | 49.40 | 44.40 |
| Xanthan gum | 0.30 | 0.30 | 0.30 | 0.30 | 0.30 |
| Methocel A4M | 0.30 | 0.30 | 0.30 | 0.30 | 0.30 |
| Minoxidil | 5.00 | 5.00 | 5.00 | 5.00 | 5.00 |
| Total | 100.00 | 100.00 | 100.00 | 100.00 | 100.00 |
| Sodium | To pH | To pH | To pH | To pH | To Ph |
| hydroxide | 4.5-5.0 | 4.5-5.0 | 4.5-5.0 | 4.5-5.0 | 4.5-5.0 |
| Propellant | 8% | 8% | 8% | 8% | 8% |
| (propane; | | | | | |
| isobutene and | | | | | |
| butane mixture) | | | | | |
| Results | | | | | |
| Appearance | | | | | |
| Quality | FG | G-E | G-E | G-E | G-E |
| Color | White | White | White | White | White |
| Odor | No odor | No odor | No odor | Very faint | No odor |
| | | | | odor | |
| Shakability | Good | Good | Good | Good | Good |
| Ph | 4.54 | 4.63 | 4.53 | 4.55 | 4.56 |
| Microscope | no | no | no | no | No |
| | crystals | crystals | crystals | crystals | crystals |

Comment:

Fairly good foam can be made in the absence of Polysorbate 60. Foam quality can be readily improved by adding effective amounts of surfactant. For example, instead of adding polysorbate 60 it is possible to achieve a good to excellent foam by using other suitable surfactants such as steareth 21. It is believed, for example, that PEG 40 stearate or sucrose esters may also be satisfactory surfactants if used in the above formulation. Polysorbate 60 can be increased from 0.1% to 25% without any substantial change in foam quality.

### Example C9

### 20% PG; Aqueous Foam Composition with 15% Polysorbate 80

| | 15 |
|---|---|
| Ingredients | |
| Propylene glycol | 20.00 |
| Polysorbate 80 | 15.00 |
| Lactic acid | 10.00 |
| Purified water | 49.40 |
| Xanthan gum | 0.30 |
| Methocel A4M | 0.30 |
| Minoxidil | 5.00 |
| Total | 100.00 |
| Sodium hydroxide | To pH |
| | 4.5-5.0 |
| Propellant (propane; | 8% |
| isobutene and butane | |
| mixture) | |
| Results | |
| Appearance | |
| Quality | G-E |
| Color | White |
| Odor | No odor |
| Shakability | Good |
| Ph | 4.52 |
| Microscope | no crystals |

Comment:

Polysorbate 60 was replaced by Polysorbate 80, which is larger. The formulation produced a good to excellent quality foam with a pleasant skin feeling.

### Example C10

### High 71% PG; Aqueous Foam Carrier Composition with Klucel, with Montanov 68 and with and without 12% Polysorbate 60

| | M010 | M011 |
|---|---|---|
| Ingredients | | |
| Propylene glycol | 71.00 | 71.00 |
| Klucel EF | 0.50 | 0.50 |
| Polysorbate 60 | 12.00 | |
| Montanov 68 | 1.00 | 5.00 |
| Purified water | 15.50 | 23.50 |
| Total | 100.00 | 100.00 |
| Propellant | 8% | 8% |
| (propane; | | |
| isobutene and | | |
| butane mixture) | | |
| Results | | |
| Appearance: | | |
| Quality | G-E | G-E |
| Color | W | W |
| Odor | No | No |

Comment:

The formulations with and without polysorbate 60 were not substantially different. Polysorbate 60 is a surfactant which is believed to aid the penetration and effect of minoxidil, probably in a synergistic manner. No crystals were observed.

### Example C11

### High 67% PG; Aqueous Foam Compositions with 12% Polysorbate 60 and Klucel with and without Cetearyl Alcohol (and) Cetearyl Glucoside

### Part-A—Formulations

| | | | M07 | M08 |
|---|---|---|---|---|
| | M05 | M06 | (=MIN1) | (=MIN2) |
| Ingredients | | | | |
| Propylene glycol | 67.00 | 67.00 | 67.00 | 67.00 |
| Klucel EF | 1.00 | 0.50 | | 0.50 |
| Klucel GF | | | 0.50 | |
| Polysorbate 60 | 12.00 | 12.00 | 12.00 | 12.00 |
| Cetearyl Alcohol | | | | 1.00 |
| (And) Cetearyl | | | | |
| Glucoside. | | | | |
| (Emulsifier) | | | | |
| Minoxidil | 5.00 | 5.00 | 5.00 | 5.00 |
| Purified water | 15.00 | 15.50 | 15.50 | 14.50 |
| Total | 100.00 | 100.00 | 100.00 | 100.00 |
| Propellant | 8% | 8% | 8% | 8% |
| (propane; | | | | |
| isobutene and | | | | |
| butane mixture) | | | | |
| Results | | | | |
| Appearance: | | | | |
| Quality | G-E | G-E | G-E | G-E |
| Color | W | W | W | W |
| Odor | No | No | No | No |

Comment:

With high levels of PG the skin feeling becomes compromised and is more greasy in nature. To compensate Cetearyl Alcohol (And) Cetearyl Glucoside surfactant was added to produce an improved skin feeling. No crystals were observed.

### Part-B

FIG. 4 is a photo micrograph of Formulation M08 (2) comprising 5% Minoxidil. The image was taken after shear forces disclosing no crystals.

### Part C—Targeting

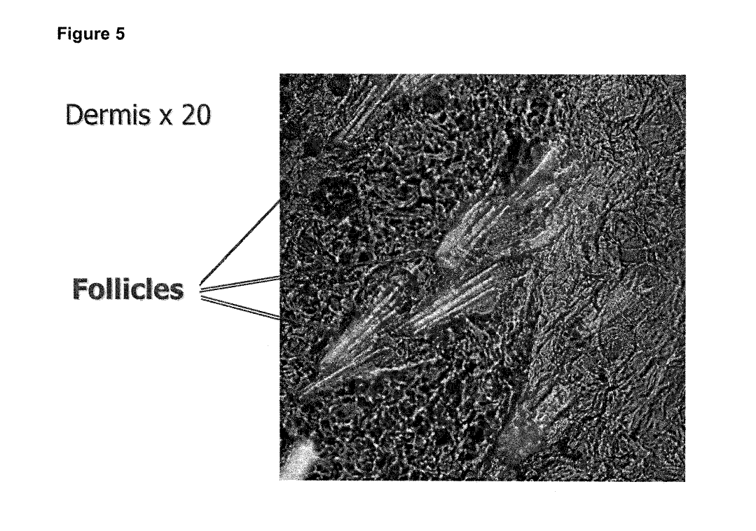

Formula MNX 008 was applied to skin sample, which was then stained and examined under the microscope and photographed. The resultant image is shown in FIG. 5 at a 20× magnificent. In the picture minoxidil appears to be concentrated in the region of the hair follicle. Thus the formula appears to be targeting the site of action.

### Part-D—Penetration

| | Average (n Repeat | | | |
|---|---|---|---|---|
| | Experiments) in | | Average in % of applied | |
| | μg/cm² | | dose | |
| | | Regaine | | Regaine |
| Formula | M08 | Forte | M08 | Forte |
| Skin | 4.80(±0.84 | 2.25(±0.32 | — | — |
| Extract | Standard | Standard | | |
| (SE¹) | Error)** | Error)** | | |
| (n = 12) | | | | |
| Receiving | 0.89(±0.18 | 0.78(±0.14 | — | — |
| Chamber | Standard | Standard | | |
| (RC) | Error)** | Error)** | | |
| (n = 6) | | | | |
| Total | 7.73(±1.09 | 3.27(±0.69 | 0.136(±0.019 | 0.058(±0.012 |
| (n = 6) | Standard | Standard | Standard | Standard |
| | Error)** | Error)** | Error)** | Error)** |

*SE¹ = is the extract of the entire skin (without removal of Tapes1, 2, and 3,) and includes the hair follicle and root.
**The statistical difference between the penetration data of these two groups was highly significant (p < 0.05).

Comment:

The penetration study results (presented as a mean of 12 repeat experiments) disclose that the penetration in the skin including hair root and follicle with 67% PG is more than double than that of the commercial hair preparation. Note the protocol for these experiments was not identical to that for the other penetration studies and therefore these results are not compared with the other penetration studies. See Methodology—Protocol A

### Example C12

### High 76% PG; Aqueous Foam Composition with Klucel and Montanov 68 but without Polysorbate 60

| | M09 |
|---|---|
| Ingredients | |
| Propylene glycol | 76.00 |
| Klucel EF | 0.50 |
| Montanov 68 | 1.00 |
| Minoxidil | 5.00 |
| Purified water | 17.50 |
| Total | 100.00 |
| Propellant | 8% |
| (propane; | |
| isobutene and | |
| butane mixture) | |
| Results | |
| Appearance: | |
| Quality | G-E |
| Color | W |
| Odor | No |

Comment:

The formulation produced a good to excellent quality foam without crystals. Penetration of this formula was compared to that of Example 11, Formula 8. No statistical difference was noted between the two formulations. See Methodology—Protocol A

### Example C12

### High 62% PG; Aqueous Foam Composition with Klucel and Montanov 68 and 12% Polysorbate 60 and 5% Ethanol

### Part A—Formulation

| | M013 |
|---|---|
| Ingredients | |
| Propylene glycol | 62.00 |
| Klucel EF | 0.50 |
| Polysorbate 60 | 12.00 |
| Ethanol | 5.00 |
| Cetearyl Alcohol | 1.00 |
| (And) Cetearyl | |
| Glucoside. | |
| Minoxidil | 5.00 |
| Purified water | 14.50 |
| Total | 100.00 |
| Propellant | 8% |
| (propane; | |
| isobutene and | |
| butane mixture) | |
| Results | |
| Quality | G-E |
| Color | W |
| Odor | No |
| Ingredients | M013 |

Comment:

The presence of 5% ethanol did not result in a significantly improved skin feeling.

### Part B—Penetration Study

Resume (12 replicates)

Average in % of applied
dose

| | M013- | Regaine Forte |
|---|---|---|
| Tape 1 | 74.24 | 73.88 |
| Tape 2 | 2.27 | 2.73 |
| Tape 3 | 0.56 | 0.37 |
| SE* | 0.27 | 0.14 |
| RC (as is) | 1.14 | 0.51 |
| Total without tape | 4.25 | 3.75 |
| 1 (Tape 2, | | |
| 3, SE and RC) | | |
| Total skin | 3.10 | 3.25 |
| (Tapes 2, 3 and SE) | | |
| Total Mass | 78.49 | 77.64 |
| balance | | |

*Tape 1 = Surface; Tape 2 = Upper Stratum Corneum; Tape 3 = Lower Stratum Corneum; SE = is the extract of the skin remaining after removal of Tapes1, 2, and 3, and includes the hair follicle root.

Comment:

The penetration study results (presented as a mean of twelve repeat experiments) disclose that the penetration in the remaining skin including hair root and follicle after removal of the surface and stratum corneum is almost double that of a commercial hair preparation, although the overall skin penetration was similar with both preparations. See Methodology—Protocol B

### Example C13

### High 55% to 67% PG; Aqueous Foam Composition with ASOS and Cetearyl Alcohol (and) Cetearyl Glucoside and 12% Polysorbate 60

| Ingredients | M014 | M015 | M016 | M017 | M018 | M019 |
|---|---|---|---|---|---|---|
| Propylene | 67.00 | 60.00 | 55.00 | 58.00 | 58.00 | 58.00 |
| glycol | | | | | | |
| ASOS | 3.00 | 3.00 | 3.00 | 3.00 | 3.00 | 3.00 |
| (aluminum | | | | | | |
| starch octenyl | | | | | | |
| succinate) | | | | | | |
| Methyl glucose | | | | 1.00 | 1.00 | 1.00 |
| sesqistearate | | | | | | |
| (MGS) | | | | | | |
| Polysorbate 60 | 12.00 | 12.00 | 12.00 | 15.00 | 15.00 | 15.00 |
| Ethanol | | | 10.00 | | | |
| Cetearyl | 1.00 | 0.50 | 0.50 | | | |
| Alcohol | | | | | | |
| (And) Cetearyl | | | | | | |
| Glucoside. | | | | | | |
| Minoxidil | 5.00 | 5.00 | 5.00 | 6.00 | 7.00 | 10.00 |
| Purified water | 12.00 | 19.50 | 14.50 | 17.00 | 16.00 | 13.00 |
| Total | 100.00 | 100.00 | 100.00 | 100.00 | 100.00 | 100.00 |
| Propellant | 8% | 8% | 8% | 8% | 8% | 8% |
| (propane; | | | | | | |
| isobutene and | | | | | | |
| butane mixture) | | | | | | |
| Results | | | | | | |
| Quality | G-E | G-E | G-E | G-E | G-E | G-E |
| Color | W | W | W | W | W | W |
| Odor | No | No | No | No | No | No |

Comment:

ASOS was added to try and improve skin feeling. MGS is a viable alternative to Cetearyl Alcohol (And) Cetearyl Glucoside. No crystals were observed.

### Example C14

### Low 10% PG; Aqueous Foam Composition with 12% Polysorbate 60

| | M03 | M04 |
|---|---|---|
| Ingredients | | |
| Propylene glycol | 10.00 | 10.00 |
| CMC | 1.50 | |
| Metocel K100M | | 0.30 |
| Xantan Gum | | 0.30 |
| Polysorbate 60 | 12.00 | 12.00 |
| Minoxidil | 5.00 | 5.00 |
| Sharomix 824 | 0.60 | 0.60 |
| Purified water | 70.90 | 71.80 |
| Total | 100.00 | 100.00 |
| Propellant | 8% | 8% |
| (propane; | | |
| isobutene and | | |
| butane mixture) | | |
| Results | | |
| Quality | G-E | G-E |
| Color | W | W |
| Odor | No | No |

Comment:

The concentration of Polysorbate 60 was reduced to 12%. To compensate, an additional surfactant Sharonmix 824 (a liquid blend of methyl paraben, ethyl paraben and propyl paraben—in phenoxyethanol) was added. The formulations produced a good to excellent quality foam with a pleasant skin feeling. Satisfactory foam may be made using lower concentrations of PG.

### Example C15

### High 77% to 80% PG; Waterless Foam Composition with 12% Polysorbate 80

### Part A—Formulation

| | M01 | M02 |
|---|---|---|
| Ingredients | | |
| Propylene glycol | 77.00 | 80.00 |
| Klucel EF | 2.00 | 1.00 |
| Laureth-4 | 2.00 | 2.00 |
| Simulsol 165 | 2.00 | 1.00 |
| Polysorbate 60 | 12.00 | 12.00 |
| Minoxidil | 5.00 | 5.00 |
| Total | 100.00 | 101.00 |
| Propellant | 8% | 8% |
| (propane; | | |
| isobutene and | | |
| butane mixture) | | |
| Results | | |
| Appearance: | | |
| Quality | G-E | G-E |
| Color | W | W |
| Odor | No | No |

Comment:

The waterless formulation produced a good to excellent quality foam without crystals.

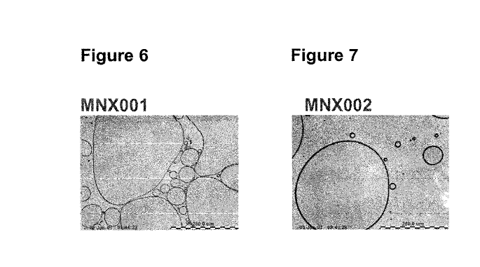

FIGS. 6 and 7 are photo micrographs of Non Aqueous Formulations M01 and M02, respectively comprising 5% Minoxidil and disclosing no crystals.

### Section D

### High Oil Emulsion; Non Comedogenic Emulsion; and Hydrophillic and Water Formulations

### Example D1

### Prophetic High Oil Content DISPA (Diisopropyl Adipate) Formulation for Solubilizing Water Insoluble Drugs and Provide High Moisturizing and Fatting Vehicle Intended Primarily for Topical Use

| | % W/W | | | | | | | | | | |
|---|---|---|---|---|---|---|---|---|---|---|---|
| | Group A | | | | Group B | | | | Group C | | |
| Ingredients | 1 | 2 | 3 | 4 | 5 | 6 | 7 | 8 | 9 | 10 | 11 |
| API | 1 | 1 | 1 | 1 | 1 | 1 | 1 | 1 | 1 | 1 | 1 |
| Diisopropyl | 20 | 20 | 20 | 20 | 20 | 20 | 20 | 20 | 20 | 20 | 20 |
| adipate | | | | | | | | | | | |
| Oleyl alcohol | 10 | 10 | 10 | 10 | 10 | 10 | 10 | 10 | 10 | 10 | 10 |
| PEG-40 Stearate | 3 | — | 3 | 3 | 3 | — | 3 | — | 3 | 3 | — |
| Polysorbate 80 | 1 | — | 1 | 1 | 1 | — | 1 | — | 1 | 1 | — |
| Steareth-21 | — | 2 | — | — | — | 2 | — | 2 | — | — | 2 |
| Steareth-2 | — | 3 | — | — | — | 3 | — | 3 | — | — | 3 |
| Laureth-4 | — | 2 | 2 | — | 2 | — | — | — | — | 2 | 2 |
| Sorbitan Stearate | — | — | — | 2 | — | 2 | 2 | — | — | — | — |
| Stearic acid | 1 | — | 1 | 1 | — | 1 | — | 1 | — | 1 | 1 |
| Glyceryl stearate | 1 | 1 | — | — | 1 | — | 1 | 1 | 1 | 1 | — |
| Carboxymethyl | — | 0.5 | 0.5 | — | — | — | — | 0.5 | — | — | — |
| cellulose sodium | | | | | | | | | | | |
| Xanthan gum | 0.35 | — | — | — | — | 0.35 | 0.35 | — | — | 0.35 | 0.35 |
| Hydroxypropyl | 0.35 | — | — | — | — | 0.35 | 0.35 | — | — | 0.35 | 0.35 |
| methyl cellulose | | | | | | | | | | | |
| (Methocel K100M) | | | | | | | | | | | |
| Dimethyl | — | — | — | — | 10 | 10 | 10 | 10 | — | — | — |
| isosorbide | | | | | | | | | | | |
| Propylene Glycol | 10 | 10 | 10 | 10 | — | — | — | — | — | — | — |
| Benzyl alcohol | 1 | 1 | 1 | 1 | 1 | 1 | 1 | 1 | 1 | 1 | 1 |
| Citric acid | 0.3 | 0.3 | 0.3 | 0.3 | 0.3 | 0.3 | 0.3 | 0.3 | 0.3 | 0.3 | 0.3 |
| Sodium Citrate | 0.75 | 0.75 | 0.75 | 0.75 | 0.75 | 0.75 | 0.75 | 0.75 | 0.75 | 0.75 | 0.75 |
| Water | 50.25 | 48.45 | 49.45 | 49.95 | 49.95 | 48.25 | 49.25 | 49.45 | 61.95 | 58.25 | 58.25 |
| Propellant | 8 | 8 | 8 | 8 | 8 | 8 | 8 | 8 | 8 | 8 | 8 |
| (propane/butane/ | | | | | | | | | | | |
| isobutene) | | | | | | | | | | | |

Group A with PG, Group B with DMI and Group C none.

Comment:

Oil in water emulsion with 20% to 40% oil phase; stabilizing surfactants 2 to 5%; foam adjutants 1 or 2%; stabilizing polymers 0% (none) to 0.7%; hydrophilic solvent 5% to 30%; water to 100% and optionally, buffer and microbial preservative.

These formulations are thought to be able to produce oil in water emulsion foam of good or excellent quality, which will be stable such that it can substantially withstand FTC procedure and or one to two weeks at 40° C.

These formulations are thought to be suitable carriers for an active agent selected from the group consisting of a channel agent; a cholinergic agent; a nitric oxide donor, or a related agent, wherein the channel agent is selected from the group consisting of a calcium channel blocker, a potassium channel agent; a sodium channel agent and a chloride channel agent;

The active agent may be increased up to about 6% of the formulation in place of water

### Example D2

### Prophetic Formulation Comprising Non Comedogenic Ingredients (Diisopropyl Adipate Capric/Caprilic Triglicerides Diethyl Sebacate) for Topical Conditions

| | % w/w | | | | | |
|---|---|---|---|---|---|---|
| | Group A | | | Group B | Group C | |
| Ingredients | 1 | 2 | 3 | 4 | 5 | 6 |
| API | 1 | 1 | 1 | 1 | 1 | 1 |
| Diisopropyl adipate | 6 | 6 | 6 | — | 6 | 6 |
| Capric/caprilic | 6 | 6 | 6 | 6 | — | — |
| triglicerides | | | | | | |
| Diethyl sebacate | — | — | — | 6 | 6 | 6 |
| Steareth-21 | 2 | 2 | — | — | 2 | — |
| Steareth-2 | 3 | 3 | — | — | 3 | — |
| PEG-40 Stearate | — | — | 3 | 3 | — | 3 |
| Polysorbate 80 | — | — | 1 | 1 | — | 1 |
| Glyceryl stearate | — | — | 1 | 1 | — | 1 |
| Stearic acid | 1 | 1 | 2 | 2 | 1 | 2 |
| Hydroxypropyl | 1.5 | 1.5 | — | — | 1.5 | — |
| cellulose (Klucel EF) | | | | | | |
| Xanthan gum | — | — | 0.35 | 0.35 | — | 0.35 |
| Hydroxypropyl | — | — | 0.35 | 0.35 | — | 0.35 |
| methyl cellulose | | | | | | |
| (Methocel K100M) | | | | | | |
| Benzyl alcohol | 1 | 1 | 1 | 1 | 1 | 1 |
| Citric acid | 0.6 | 0.3 | 0.3 | 0.3 | 0.3 | 0.3 |
| Sodium Citrate | 1.5 | 0.75 | 0.75 | 0.75 | 0.75 | 0.75 |
| Polyethylene Glycol | 40 | 30 | 40 | 40 | 30 | 30 |
| 400 | | | | | | |
| Glycerin | — | 8 | — | — | 8 | 8 |
| Sodium PCA | — | 2 | — | — | 2 | 2 |
| Purified water | 36.4 | 37.45 | 37.25 | 37.25 | 37.45 | 37.25 |
| Propellant (propane/ | 8 | 8 | 8 | 8 | 8 | 8 |
| butane/isobutene) | | | | | | |

Group A DISPA and MCT oil, Group B MCT oil and diethyl sebacate and Group C DISPA and diethyl sebacate.

Comment:

Oil in water emulsion with about 2% to about 30% of oil phase, about 1% to about 5% surfactant, about 0.2% to 1.5% stabilizing polymer, about 20% to about 60% of an hydrophilic solvent such as PEG400 and water to 100% and optionally microbial preservative and buffer

These formulations are thought to be suitable carriers for an active agent selected from the group consisting of a channel agent; a cholinergic agent; a nitric oxide donor, or a related agent, wherein the channel agent is selected from the group consisting of a calcium channel blocker, a potassium channel agent; a sodium channel agent and a chloride channel agent.

### Example D3

### Prophetic Formulation without Oils Comprising Hydrophilic Solvents and Water

Group A with propylene glycol, Group B with Ethanol and Group C none.

| | % w/w | | | | | | | | | | |
|---|---|---|---|---|---|---|---|---|---|---|---|
| | Group A | | | | Group B | | | Group C | | | |
| Ingredients | 1 | 2 | 3 | 4 | 5 | 6 | 7 | 8 | 9 | 10 | 11 |
| API | 1 | 1 | 1 | 1 | 1 | 1 | 1 | 1 | 1 | 1 | 1 |
| Purified water | 40 | 40 | 40 | 40 | 40 | 40 | 40 | 15 | 40 | 40 | 40 |
| Ethyl alcohol | — | — | — | — | 15 | 15 | 15 | — | — | — | — |
| Propylene | 15 | 15 | 15 | 15 | — | — | — | — | — | — | — |
| Glycol | | | | | | | | | | | |
| Steareth-21 | 1 | 1 | 1.5 | 1.5 | 1 | 1.5 | 0.35 | 2 | 1.5 | 0.35 | 1.5 |
| Steareth-2 | 1.5 | 1.5 | 2.5 | 2.5 | 1.5 | 2.5 | 0.65 | 3 | 2.5 | 0.65 | 2.5 |
| Carboxymethyl | 0.5 | 0.5 | — | — | — | 0.5 | 0.5 | — | — | 0.5 | — |
| cellulose | | | | | | | | | | | |
| sodium | | | | | | | | | | | |
| Hydroxypropyl | — | — | 1 | 1 | 1 | — | — | 1.5 | 1 | — | 1 |
| cellulose | | | | | | | | | | | |
| (Klucel EF) | | | | | | | | | | | |
| Benzyl alcohol | 1 | 1 | 1 | 1 | 1 | 1 | — | 1 | 1 | 1 | 1 |
| Citric acid | To pH | 0.3 | To pH | To pH | 0.3 | 0.3 | To pH | 0.3 | 0.3 | 0.3 | 0.3 |
| | 5.0 | | 5.0 | 5.0 | | | 5.0 | | | | |
| Sodium Citrate | — | 0.75 | — | — | 0.75 | — | 0.75 | 0.75 | 0.75 | 0.75 | 0.75 |
| Polyethylene | 40 | 38.95 | 38 | — | 38.45 | 37.45 | 41.5 | 65 | 51.95 | 55.45 | 12.95 |
| Glycol 400 | | | | | | | | | | | |
| Polyethylene | — | — | — | 38 | — | — | — | 11.45 | — | — | 39 |
| Glycol 200 | | | | | | | | | | | |
| Propellant | 8 | 8 | 8 | 8 | 8 | 8 | 8 | 8 | 8 | 8 | 8 |
| (propane/ | | | | | | | | | | | |
| butane/ | | | | | | | | | | | |
| isobutene) | | | | | | | | | | | |

Comment:

The formulation is a homogeneous mixture of 30% to 70% hydrophilic solvents; alcohols and polyols (PEG200, PEG400, ethanol, Propylene glycol), and water to 100% and 1% to 5% stabilizing surfactants, 0.5% to 2.0% polymer, and optionally buffer and microbial preservative

These formulations are thought to be able to produce foam of good or excellent quality, which will be stable such that it can substantially withstand FTC procedure and or one to two weeks at 40° C. They are designed to produce foams with non greasy feeling that are absorbed into the skin quickly for solubilizing water insoluble drugs. They are single phase formulations.

These formulations are thought to be suitable carriers for an active agent selected from the group consisting of a channel agent; a cholinergic agent; a nitric oxide donor, or a related agent, wherein the channel agent is selected from the group consisting of a calcium channel blocker, a potassium channel agent; a sodium channel agent and a chloride channel agent.

The active agent may be increased up to about 6% of the formulation in place of water.

1. The foamable therapeutic composition, comprising:
i. a therapeutically effective concentration of at least one active agent selected from the group consisting of a channel agent, a cholinergic agent, and a nitric oxide donor; and
ii. a foamable carrier comprising:
a. about 50% to about 98% of a solvent selected from the group consisting of water; a hydrophilic solvent; a hydrophobic solvent; a potent solvent; a polar solvent, a silicone, an emollient, and mixtures thereof;
b. 0% to about 48% of a secondary solvent selected from the group consisting of water; a hydrophilic solvent; a hydrophobic solvent; a potent solvent; a polar solvent, a silicone, an emollient, a co-solvent, a penetration enhancer and mixtures thereof;
c. a surface-active agent;
d. about 0% to about 5% by weight of at least one polymeric agent; and
e. a liquefied or compressed gas propellant at a concentration of about 3% to about 25% by weight of the total composition;
wherein the composition is housed in a container and is substantially flowable and which upon release expands to form a breakable foam; and
wherein the foamable carrier is selected to generate a foam of good to excellent quality.

2.-53. (canceled)
